(19) United States
(12) Patent Application Publication
(10) Pub. No.: US 2006/0014248 A1
(43) Pub. Date: Jan. 19, 2006

(54) TNF super family members with altered immunogenicity

(75) Inventors: Shannon Alicia Marshall, San Francisco, CA (US); Gregory L. Moore, Pasadena, CA (US); Arthur J. Chirino, Camarillo, CA (US); John R. Desjarlais, Pasadena, CA (US)

Correspondence Address: DORSEY & WHITNEY LLP, 555 CALIFORNIA STREET, SUITE 1000, SUITE 1000, SAN FRANCISCO, CA, 94104, US

(73) Assignee: Xencor, Inc.

(21) Appl. No.: 11/136,079

(22) Filed: May 23, 2005

Related U.S. Application Data
(63) Continuation-in-part of application No. 10/794,751, filed on Mar. 5, 2004.
(63) Continuation-in-part of application No. 10/338,785, filed on Jan. 6, 2003.
(63) Continuation-in-part of application No. 10/820,465, filed on Mar. 31, 2004.
(60) Provisional application No. 60/573,206, filed on May 21, 2004.
(60) Provisional application No. 60/573,301, filed on May 21, 2004.
(60) Provisional application No. 60/573,395, filed on May 21, 2004.
(60) Provisional application No. 60/588,314, filed on Jul. 14, 2004.
(60) Provisional application No. 60/607,396, filed on Sep. 2, 2004.
(60) Provisional application No. 60/607,397, filed on Sep. 2, 2004.
(60) Provisional application No. 60/452,707, filed on Mar. 7, 2003.
(60) Provisional application No. 60/482,081, filed on Jun. 23, 2003.
(60) Provisional application No. 60/459,094, filed on Mar. 31, 2003.
(60) Provisional application No. 60/510,430, filed on Oct. 10, 2003.
(60) Provisional application No. 60/517,728, filed on Nov. 5, 2003.
(60) Provisional application No. 60/523,545, filed on Nov. 20, 2003.

(51) Int. Cl. C12P 21/06; C07H 21/04; C07K 14/525

(52) U.S. Cl. 435/69.1; 435/320.1; 435/325; 530/351; 536/23.5

(57) ABSTRACT

The present invention relates to non-naturally occurring variant Tumor Necrosis Factor Super Family member proteins with reduced immunogenicity. More specifically, the present invention relates to variant BAFF, RANKL, TRAIL, CD40L and APRIL proteins with reduced immunogenicity.

This application claims benefit under 35 U.S.C. §119(e) to U.S. Ser. Nos 60/573,206, filed May21, 2004; 60/573,301, filed May 21, 2004; 60/573,395, filed May 21, 2004; 60/588,314, filed Jul. 14, 2004; 60/607,396, filed Sep. 2, 2004; and, 60/607,397, filed Sep. 2, 2004; and is a continuation in part of Ser. No. 10/794,751, filed Mar. 4, 2004, which claims benefit under 35 U.S.C. §119(e) to 60/452,707, file Mar. 7, 2003 and 60/482,081, filed Jun. 23, 2003; and is a continuation in part of Ser. No. 10/338,785 Jan. 6, 2003; and is a continuation in part of Ser. No. 10/820,465, filed Mar. 31, 2004, which claims benefit under 35 U.S.C. §119(e) to 60/459,094, filed Mar. 31, 2003; and 60/510,430, filed Oct. 10, 2003, 60/517,728, filed Nov. 5, 2003, and 60/523,545, filed Nov. 20, 2003; all entirely incorporated by reference.

### FIELD OF THE INVENTION

The present invention relates to variant Tumor Necrosis Factor Super Family member proteins with reduced immunogenicity. More specifically, the present invention relates to variant BAFF, RANKL, TRAIL, CD40L and APRIL proteins with reduced immunogenicity. In particular, variants of BAFF, RANKL, TRAIL, CD40L and APRIL proteins with reduced ability to bind one or more human class II MHC molecules are described.

### BACKGROUND OF THE INVENTION

Immunogenicity is a major barrier to the development and utilization of protein therapeutics. Although immune responses are typically most severe for non-human proteins, even therapeutics based on human proteins may be immunogenic. Immunogenicity is a complex series of responses to a substance that is perceived as foreign and may include production of neutralizing and non-neutralizing antibodies, formation of immune complexes, complement activation, mast cell activation, inflammation, and anaphylaxis. Several factors can contribute to protein immunogenicity, including but not limited to the protein sequence, the route and frequency of administration, and the patient population.

Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. One special class of side effects results when neutralizing antibodies cross-react with an endogenous protein and block its function.

Several methods have been developed to modulate the immunogenicity of proteins. In some cases, PEGylation has been observed to reduce the fraction of patients who raise neutralizing antibodies by sterically blocking access to antibody agretopes (see for example, Hershfield et. al. PNAS 1991 88:7185-7189 (1991); Bailon. et al. Bioconjug. Chem. 12: 195-202(2001); He et al. Life Sci. 65: 355-368 (1999), entirely incorporated by reference). Methods that improve the solution properties of a protein therapeutic may also reduce immunogenicity, as aggregates have been observed to be more immunogenic than soluble proteins.

A more general approach to immunogenicity reduction involves mutagenesis targeted at the agretopes in the protein sequence and structure that are most responsible for stimulating the immune system. Some success has been achieved by randomly replacing solvent-exposed residues to lower binding affinity to panels of known neutralizing antibodies (see for example Laroche et. al. Blood 96: 1425-1432 (2000), entirely incorporated by reference). Due to the incredible diversity of the antibody repertoire, mutations that lower affinity to known antibodies will typically lead to production of an another set of antibodies rather than abrogation of immunogenicity. However, in some cases it may be possible to decrease surface antigenicity by replacing hydrophobic and charged residues on the protein surface with polar neutral residues (see Meyer et. al. Protein Sci. 10: 491-503 (2001), entirely incorporated by reference).

An alternate approach is to disrupt T-cell activation. Removal of MHC-binding agretopes offers a much more tractable approach to immunogenicity reduction, as the diversity of MHC molecules comprises only ˜103 alleles, while the antibody repertoire is estimated to be approximately 108 and the T-cell receptor repertoire is larger still. By identifying and removing or modifying class II MHC-binding peptides within a protein sequence, the molecular basis of immunogenicity can be evaded. The elimination of such agretopes for the purpose of generating less immunogenic proteins has been disclosed previously; see for example WO 98/52976, WO 02/079232, and WO 00/3317, entirely incorporated by reference.

While mutations in MHC-binding agretopes can be identified that are predicted to confer reduced immunogenicity, most amino acid substitutions are energetically unfavorable. As a result, the vast majority of the reduced immunogenicity sequences identified using the methods described above will be incompatible with the structure and/or function of the protein. In order for MHC agretope removal to be a viable approach for reducing immunogenicity, it is crucial that simultaneous efforts are made to maintain a protein's structure, stability, and biological activity.

B-cell Activation Factor, BAFF (also known as BLyS, TALL-1, THANK, zTNF4 and TNFSF13B) is a member of the TNF super family (TNFSF) of proteins. BAFF is important for survival of B-cells and humoral immune response; to a lesser extent it also induces T-cell activation and proliferation. Normally, only a small number of B-cells mature due to a vigorous negative selection. Overexpression of BAFF in transgenic (Tg) animals promotes increased B-cell survival, resulting in inappropriate survival of autoreactive lymphocytes and enlarged lymphoid organs and spleen, accompanied by the appearance of anti-DNA antibodies, an increase in antibody secretion, and Ig-deposition in the kidneys. This results in glomerulonephritis and syndromes similar to systemic lupus erythematosus (SLE), Sjogren syndrome (SS), and the like. Correlations between high BAFF concentration and elevated levels of anti-dsDNA Ab, a biochemical marker of several autoimmune diseases, have been observed in SLE, RA, and SS patients.

BAFF binds three receptors: BAFF-R, TACI, and BCMA. BAFF-R is specific to BAFF while TACI and BCMA are shared with APRIL, another member of TNFSF and the closest homologue of BAFF. Phenotypes of BAFF knockout mice (KO) and a BAFF-R mutant strain of mice (A/WySnJ) suggest that BAFF-R is the main receptor for BAFF and is responsible for control of B-cell maturation. TACI controls B-cell homeostasis and T-cell Independent immune response and appears to act as an inhibitory BAFF receptor. The role of BCMA is unclear thus far.

BAFF is an attractive drug target because it has been implicated in the pathogenesis of several diseases and because BAFF inhibitors would potentially have few side effects. A previous invention provided variant BAFF proteins that function as dominant negative or competitive inhibitors of endogenous BAFF. Furthermore, superagonist variants of BAFF were generated, which may serve to stimulate the immune system. BAFF and variant BAFF proteins, like all proteins, has the potential to induce unwanted immune responses when used as a therapeutic. Accordingly, the development of therapeutics based on BAFF may be facilitated by preemptively reducing the potential immunogenicity of BAFF or its variants.

RANKL is a trimeric TNF family member that binds to the trimeric RANK receptor. RANKL is a key modulator of bone remodeling orchestrated by osteoblasts and osteoclasts. (See US 2003/0013651 and WO 02/080955, entirely incorporated by reference). RANKL activates the receptor RANK upon binding, which leads to the differentiation, survival, and fusion of pre-osteoclasts to form active bone resorbing osteoclasts (see Lacey D L, Timms E, Tan H-L, Kelley M J, Dunstan C R, Burgess T et al. 1998 Cell 93: p. 165-176, entirely incorporated by reference). RANKL also binds to the decoy receptor OPG, which functions as a natural antagonist of RANKL activity.

The RANKL biochemical axis has been successfully targeted to treat osteoporosis, rheumatoid arthritis, prosthesis-induced osteolysis, cancer-induced bone destruction, metastasis, hypercalcemia, and pain (Hofbauer et. al. 2001 Cancer 92:460-470; Takahashi et.al. 1999 Biochem. Biophys. Res. Comm. 256:449-455; Honore et al. 2000 Nat. Med. 6:521-528; Oyajobi et.al. 2001 Cancer Res 61:2572-2578; Childs et. el. J. Bone Mineral Res. 17:192-199, entirely incorporated by reference). In addition to being important in bone biology, RANKL plays a role in the immune system by regulating antigen-specific T cell responses (Anderson et al., Nature 1997, 390:175-179, entirely incorporated by reference).

Much work has been done to develop therapeutic entities and reagents for biological research based on RANKL. For example, RANKL fragments, analogs, derivatives, or conformers having the ability to bind OPG, which could be used as treatments for a variety of bone diseases, have been described (See U.S. Pat. No. 5,843,678, entirely incorporated by reference). RANKL variants, which induce production of an immune response that down-regulates RANKL activity, have been disclosed (See WO00/15807). In other studies, utilization of RANKL protein and its derivatives as immune modulators has been proposed (See WO99/29865, entirely incorporated by reference). Novel RANKL variants, including variants that express solubly in E. coli, dominant negative variants, competitive inhibitor variants, receptor-specific variants, and superagonist variants have been disclosed (U.S. Ser. No. 10/338,785, filed Jan. 6, 2003, entirely incorporated by reference.)

A PRoliferation-Inducing Ligand (APRIL), also known as TRDL-1 alpha, TALL-2, and TNFSF-13A, is a member of the TNF Super Family (TNFSF) of proteins. The prototype of the family, Tumor Necrosis Factor Alpha (TNFα), originally discovered for promoting tumor regression in vivo, is a key mediator of inflammation. APRIL also participates in a variety of cellular and intracellular signaling processes involved in autoimmune disease, inflammation, and cancer.

APRIL is expressed by macrophages, monocytes, dendritic cells, T cells, and a number of human tumors and transformed cell lines. It is synthesized as a type II transmembrane protein, cleaved intracellularly in the Golgi apparatus by a furin convertase, and secreted predominantly as a soluble molecule. A splice variant of the APRIL/TWEAK locus also exists, which results in a functional hybrid molecule (TWE-PRIL) that is primarily retained on the cell surface (Lopez-Fraga et al. EMBO Rep 2: 945-951 (2001), entirely incorporated by reference). Structurally, APRIL is a sandwich of two anti-parallel beta-sheets with the “jelly roll” or Greek key topology and forms homotrimers typical of the TNFSF. In addition, APRIL can also form heterotrimers with BAFF, another member of the TNFSF that is closely related to APRIL.

APRIL and BAFF share two common receptors, B-cell maturation antigen (BCMA) and transmembrane activator and CAML interactor (TACI). BCMA preferentially binds APRIL versus BAFF. BCMA and TACI are type III transmembrane proteins, lacking N-terminal signal sequences. The receptors are expressed on B cells and TACI has also been detected on the surface of some T cells. TACI controls B-cell homeostasis and T-cell independent immune response and may act as an inhibitory BAFF receptor. Injection of TACI-Ig strongly inhibited or prevented collagen induced arthritis in mice. The role of BCMA is unclear thus far. BCMA and TACI contain intracellular TRAF binding motifs. The signaling mechanisms of these receptors are not fully characterized; however, they appear to mediate activation of the NF-kB, p38, mitogen-activated protein kinase, JNK, AP-1 and NF-AT pathways. APRIL signaling through BCMA and TACI is triggered by binding in its oligomeric (for the most part, trimeric) form.

Existence of a third APRIL receptor is suggested from work with mouse NIH 3T3 fibroblasts: these cancer cells express no TACI or BCMA, yet APRIL overexpression stimulates their proliferation in vitro and tumorigenicity in vivo. In a similar assay, BAFF has no effect on tumor cells. Also, soluble BCMA, which can bind and block APRIL, inhibits cancer cell growth (Rennert et al. J Exp Med 192: 1677-1684 (2000), entirely incorporated by reference.) Taken together, these facts suggest the existence of a specific APRIL receptor that has not yet been identified.

APRIL costimulates B cell proliferation and IgM production and appears to play a role in T-independent type II antigen responses and T cell survival. Accordingly, APRIL may be involved in the pathogenesis of autoimmune and inflammatory conditions. APRIL serum levels inversely correlated with disease in patients with systemic lupus erythematosus (SLE), indicating that APRIL may serve as a down modulator of serological and/or clinical autoimmunity. A polymorphism in the APRIL gene has been associated with SLE (67G allele). See for example Tan et al. Arthritis Rheum 48: 982-992 (2003), Roschke et al. J Immunol 169: 4314-4321 (2002), Stohl et al. Ann Rheum Dis 63: 1096-1103 (2004), Koyama et al. Rheumatology (Oxford) 42: 980-985 (2003), all entirely incorporated by reference.

APRIL can also induce proliferation/survival of nonlymphoid cells. Elevated expression of APRIL has been found in some tumor cell lines and tumor tissue libraries. Moreover, APRIL-transfected NIH-3T3 cells show an increased rate of tumor growth in nude mice compared with the parental cell line. APRIL can also protect glioma cells against FasL- and TRAIL-induced apoptosis. These findings suggest that APRIL may be involved in the regulation of tumor cell growth. See Mackay and Ambrose Cytokine Growth Factor Rev 14: 311-324 (2003), Medema et al. Cell Death Differ 10: 1121-1125 (2003), entirely incorporated by reference.

APRIL agonists or antagonists may thus be useful in the treatment of oncological, autoimmune, and inflammatory conditions. For example, engineered variants that act as dominant-negative inhibitors, competitive inhibitors, receptor-specific agonists, or superagonists may be used U.S. Ser. No. 10/820,465, entirely incorporated by reference.

CD40L, also known as CD154, TNFSF5, TRAP, and gp39, is a member of the TNF superfamily and may trimerize to bind and activate CD40, as well as alpha IIb-beta3 integrin. CD40L is a type II membrane glycoprotein of about 33-kDa; the full-length version has 261 amino acids and the extracellular domain (ECD) comprises amino acids 45-261. In some physiological contexts, CD40L is proteolytically processed to yield a soluble form comprising amino acids 113-261. Elevated levels of this soluble form have been established for a variety of disease conditions, including but not limited to: chronic renal failure, diabetes, inflammatory bowel disease, autoimmune thrombocytopenic purpura, Hodgkin's disease, rheumatoid vasculitis, systemic lupus erythrematosis, chronic lymphocytic leukaemia, preeclampsia, sickle cell anemia, atherosclerosis, and numerous cardiovascular conditions. Elevated levels of soluble CD40L have also become well established as a reliable predictor of cardiovascular events.

CD40L is transiently expressed after MHC/peptide-induced TCR activation on CD4+T cells. These cells mediate a signal to B cells through the CD40-CD40L interaction, which results in B cell activation. Effects of B cell activation include antibody isotype switching, rescue from apoptosis, germinal center formation, B-cell differentiation and proliferation, and IgE secretion. Mutations in CD40L cause X-linked hyper IgM syndrome, which causes severe immunodeficiency, low levels of IgA, IgG, and IgE, and inability to mount a thymus-dependent humoral response. See Seyama et al. J Biol Chem 274: 11310-11320 (1999), Sacco et al. Cancer Gene Ther 7: 1299-1306 (2000), entirely incorporated by reference. The pleiotropic immunologic effects of CD40-CD40L interactions include autoimmunity, transplantation and allograft rejection, as well as control of infection.

CD40 and CD40L are also expressed in other cell types including dendritic cells, monocytes, macrophages, endothelial cells, and fibroblasts, and are involved in many inflammatory processes including leukocyte adhesion and migration, induction of chemokines and cytokines, and activation of fibroblasts and platelets. CD40L has been implicated in the pathogenesis of atherosclerosis; it promotes microglial activation and may play a role in the development of Alzheimer's disease. Activation of the CD40-CD40L system also has remarkable antitumor and antimetastatic effects on certain carcinomas. See Laman et al. Crit Rev Immunol 16: 59-108 (1996), Lutgens and Daemen Trends Cardiovasc Med 12: 27-32 (2002), Tan et al. Curr Opin Pharmacol 2: 445-451 (2002), Prasad et al. Curr Opin Hematol 10: 356-361 (2003), Tolba et al. Cancer Res 62: 6545-6551 (2002), entirely incorporated by reference.

CD40L has many potential therapeutic indications: anti-tumor or oncological conditions, including Hodgkins and non-Hodgkins lymphomas (NHL), pre- and post-transplantation immunosuppression, psoriasis, rheumatoid and collagen-induced arthritis, multiple sclerosis, systemic lupus erythematosus (SLE), allergic encephalitis, acute and chronic graft versus host disease, Crohn's disease, diabetes, chronic renal failure, mixed connective tissue disease, sickle cell anemia, inflammatory bowel disease, Hodgkin disease, rheumatoid vasculitis, chronic lymphocytic leukaemia, preeclampsia, Alzheimer's disease, and cardiovascular conditions including atherosclerosis, thrombocytopenia (Purpura), etc.

Anti-CD40L monoclonal antibodies have shown promise in animal models for the treatment of several chronic inflammatory diseases, autoimmune diseases, and in allograft and transplant rejection. However, clinical experience with CD40L (including monoclonal antibodies) has not yet produced an effective therapeutic. See Dumont Curr Opin Investig Drugs 3: 725-734 (2002), entirely incorporated by reference, discussing monoclonal antibody IDEC-131; also the Biogen/Columbia University monoclonal antibody ruplizumab (anti-CD40L) Phase II Antova trial was discontinued due to thrombo-embolic adverse effects. Recently, evidence has accumulated indicating that CD40L can activate platelets by signaling through alpha IIb-beta3 integrin (see for example Prasad et al. Proc Natl Acad Sci USA 100: 12367-12371 (2003), entirely incorporated by reference.)

TNF-related apoptosis inducing ligand (TRAIL), also known as Apo2L and TNFSF10, is a type II (intracellular N terminus and extracellular C terminus) transmembrane protein whose extracellular domain can be proteolytically cleaved at the cell surface to form a soluble ligand (residues 114-281). A member of the TNF superfamily, its extracellular domain shares sequence homology with other family members including Fas ligand, TNF-α, lymphotoxin-α, and lymphotoxin-β. Like most other TNF family members, TRAIL forms a homotrimer that binds three receptor molecules, each at the interface between two of its subunits.

Although TRAIL mRNA has been found in a variety of tissues and cells (Wiley et al. Immunity 3: 673-682 (1995)), studies with anti-mTRAIL mAb suggest that only some liver natural killer cells express TRAIL constitutively. However, TRAIL is highly expressed on most natural killer cells after stimulation with IL-2, interferons (IFNs), or IL-15; type I IFN-activated peripheral blood T cells, CD11c+ DC, and monocytes also express TRAIL (see for example Smyth et al. Immunity 18: 1-6 (2003), entirely incorporated by reference).

Soluble recombinant TRAIL selectively induces apoptosis of a variety of tumor cells and transformed cells, but not most normal cells, and has therefore gained interest as a promising cancer therapeutic, alone or in combination with other cancer treatments. Also, administration to experimental animals including mice and primates produces significant tumor regression without systemic toxicity. TRAIL can induce apoptosis regardless of p53 status, and may be particularly useful in cells where the p53 pathway has been inactivated, thus helping to circumvent resistance to chemo- and radiotherapy. See for example Griffith and Lynch Curr Opin Immunol 10: 559-563 (1998), Ashkenazi et al. J Clin Invest 104: 155-162 (1999), Almasan and Ashkenazi Cytokine Growth Factor Rev 14: 337-348 (2003), Smyth et al. Immunity 18: 1-6 (2003), Wang and El-Deiry Oncogene 22: 8628-8633 (2003), entirely incorporated by reference.

TRAIL induces apoptosis through engagement of its death receptors. At least five receptors for TRAIL have been identified in humans. Four are membrane receptors that belong to the TNF receptor family, and two of these, DR4 (TRAIL-R1) and DR5 (apo2, TRAIL-R2) are capable of transducing an apoptotic signal. The other receptors, DcR1 (TRAIL-R3), DcR2 (TRAIL-R4), and a soluble receptor called osteoprotegerin (OPG) lack death domains, but may serve as decoy receptors that inhibit TRAIL-mediated cell death when overexpressed. Most studies suggest DR5 signals through a FADD- and caspase-8-dependent pathway (Bodmer et al. Nat Cell Biol 2: 241-243 (2000)). The Bcl-2 family member Bax is required for TRAIL-induced apoptosis of certain cancer cell lines, and Bax mutation in mismatch repair-deficient tumors can cause resistance to TRAIL therapy, but preexposure to chemotherapy can rescue tumor sensitivity. While mRNA expression of TRAIL death receptors is widely distributed in both normal and malignant tissues (Chaudhary et al. Immunity 7: 821-830 (1997), entirely incorporated by reference), cell surface expression of DR5 has been reported to be elevated in malignant tumor cells. Antibodies that immunospecifically bind to TRAIL receptors, particularly DR4 or DR5, have been shown to induce apoptosis in human tumor cells and are being investigated as potential therapeutics either alone or in combination with other anticancer drugs (see Alderson et al. Proc Amer Assoc Cancer Res 44: Abs 963 (2003), Kaliberov et al. Gene Ther 11: 658-667 (2004), Patents WO-2004016753, WO-03054216, WO-03042367, WO-03038043, WO-02097033, WO-02094880, WO-02085946, WO-02079377, WO-00183560, WO-00067793, WO-00066156, WO-00048619, WO-00051638, WO-09912963, WO-09909165, WO-09907408, WO-09903992, and WO-03037913), entirely incorporated by reference.

Despite pursuit of TRAIL as a selective anticancer therapeutic, little is known about the natural physiological function of TRAIL. TRAIL appears to play an important role in both T-cell and natural killer cell-mediated tumor surveillance and suppression of tumor metastasis, and in anti-viral immune surveillance, often augmented by IFN-regulated induction (Smyth et al. J Exp Med 193: 661-670 (2001), Takeda et al. J Exp Med 195: 161-169 (2002), Almasan et al. Cytokine Growth Factor Rev 14: 337-348 (2003), entirely incorporated by reference). TRAIL also has immunosuppressive and immunoregulatory functions that may be protective against autoimmune disorders including diabetes, rheumatoid arthritis, and multiple sclerosis (Song et al. J Exp Med 191: 1095-1104 (2000), Hilliard et al. J Immunol 166: 1314-1319 (2001), Lamhamedi-Cherradi et al. Diabetes 52: 2274-2278 (2003), Lamhamedi-Cherradi et al. Nat Immunol 4: 255-260 (2003), Patent WO-2004001009 (2003), Patent WO-2004039395 (2004), entirely incorporated by reference. It has been suggested that TRAIL inhibits autoimmune inflammation by blocking cell cycle progression of activated T-cells or by inhibiting cytokine production (Song et al. J Exp Med 191: 1095-1104 (2000), entirely incorporated by reference).

TRAIL has also been reported to play a critical role in inducing hepatic cell death and hepatic inflammation (Zheng et al. J Clin Invest 113: 58-64 (2004), entirely incorporated by reference); thus, TRAIL blockers may be useful in the treatment of hepatitis and other liver diseases. TRA-8, an agonistic monoclonal antibody that binds DR5 but not other TRAIL receptors, is tumoricidal in vitro and in vivo, but unlike TRAIL, does not induce apoptosis of normal hepatocytes; this suggests that specific targeting of DR5 may be a safe and effective strategy for cancer therapy (see Ichikawa et al. Nat Med 7: 954-960 (2001), entirely incorporated by reference). The specific targeting of DR5 on the highly proliferative synovial cells has also been suggested as a potential therapy for rheumatoid arthritis (see Ichikawa et al. J Immunol 171: 1061-1069 (2003), entirely incorporated by reference).

Daily iv injections of 0.1 to 10 mg/kg soluble human TRAIL in cynomolgus monkeys for 7 days elicited no detectable anti-TRAIL antibodies, suggesting that TRAIL is not highly immunogenic (see Ashkenazi et al. J Clin Invest 104: 155-162 (1999), entirely incorporated by reference); similarly no anti-TRAIL antibodies were detected in chimpanzees 14 days post injection of 1-5 mg/kg TRAIL iv (Kelley et al. J Pharmacol Exp Ther 299: 31-38 (2001), entirely incorporated by reference). However TRAIL, like all proteins, has the potential to induce unwanted immune responses when used as a therapeutic. Accordingly, the development of therapeutics based on TRAIL may be facilitated by pre-emptively reducing the potential immunogenicity of TRAIL.

TNF Super Family members, like all proteins, has the potential to induce unwanted immune responses when used as a therapeutic. Accordingly, the development of therapeutics based on TNF Super Family members may be facilitated by preemptively reducing the potential immunogenicity of TNF Super Family members. There remains a need for novel TNF super family member proteins, including but not limited to superagonist, dominant negative, and competitive inhibitor variant TNF super family member proteins, having reduced immunogenicity.

### SUMMARY OF THE INVENTION

In accordance with the objects outlined above, the present invention provides novel TNF Super Family member proteins having reduced immunogenicity as compared to naturally occurring TNF Super Family member proteins. In an additional aspect, the present invention is directed to methods for engineering or designing less immunogenic proteins with TNF Super Family member activity for therapeutic use.

An aspect of the present invention are TNF Super Family member variants that show decreased binding affinity for one or more class II MHC alleles relative to a parent TNF Super Family member and which significantly maintain the activity of native naturally occurring TNF Super Family member. In a further aspect, the invention provides recombinant nucleic acids encoding the variant TNF Super Family member proteins, expression vectors, and host cells. In an additional aspect, the invention provides methods of producing a variant TNF Super Family member protein comprising culturing the host cells of the invention under conditions suitable for expression of the variant TNF Super Family member protein.

In a further aspect, the invention provides pharmaceutical compositions comprising a variant TNF Super Family member protein or nucleic acid of the invention and a pharmaceutical carrier. In a further aspect, the invention provides methods for preventing or treating TNF Super Family member responsive disorders comprising administering a variant TNF Super Family member protein or nucleic acid of the invention to a patient.

In an additional aspect, the invention provides methods for screening the class II MHC haplotypes of potential patients in order to identify individuals who are particularly likely to raise an immune response to a wild type or variant TNF Super Family member therapeutic.

In accordance with the objects outlined above, the present invention provides TNF Super Family member variant proteins comprising amino acid sequences with at least one amino acid insertion, deletion, or substitution compared to the wild type TNF Super Family member proteins.

### BRIEF DESCRIPTION OF THE DRAWINGS

FIG. 1 shows a method for engineering less immunogenic BAFF derivatives.

FIG. 2 shows a schematic representation of a method for in vitro testing of the immunogenicity of BAFF peptides or proteins with IVV technology.

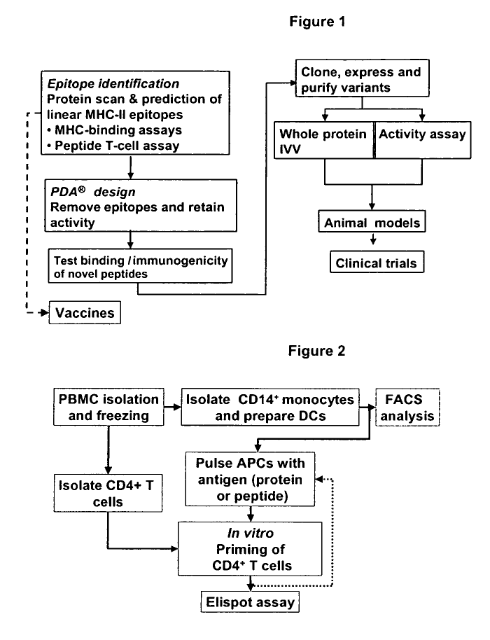

### DETAILED DESCRIPTION OF THE INVENTION

By “9-mer peptide frame” and grammatical equivalents herein is meant a linear sequence of nine amino acids that is located in a protein of interest. 9-mer frames may be analyzed for their propensity to bind one or more class II MHC alleles. By “allele” and grammatical equivalents herein is meant an alternative form of a gene. Specifically, in the context of class II MHC molecules, alleles comprise all naturally occurring sequence variants of DRA, DRB1, DRB3/4/5, DQA1, DQB1, DPA1, and DPB1 molecules. By “TNF Super Family member responsive disorders or conditions” and grammatical equivalents herein is meant diseases, disorders, and conditions that can benefit from treatment with TNF Super Family member proteins. Examples of TNF Super Family member-responsive disorders include, but are not limited to, autoimmune diseases such as systemic lupus erythematosus, diabetes, rheumatoid arthritis, ankylosing spondylitis, inflammatory bowel disease, Crohn's Disease, and psoriasis; transplant rejection and graft versus host disease; hematological cancers such as Hodgkin's lymphoma, non-Hodgkin's lymphomas (Burkitt's lymphoma, small lymphocytic lymphoma/chronic lymphocytic leukemia, mycosis fungoides, mantle cell lymphoma, follicular lymphoma, diffuse large B-cell lymphoma, marginal zone lymphoma, hairy cell leukemia and lymphoplasmacytic leukemia), tumors of lymphocyte precursor cells (B-cell acute lymphoblastic leukemia/lymphoma and T-cell acute lymphoblastic leukemia/lymphoma), tumors of the mature T and NK cells (peripheral T-cell leukemias, adult T-cell leukemia/T-cell lymphomas and large granular lymphocytic leukemia), Langerhans cell histocytosis, myeloid neoplasias (acute myelogenous leukemias), and chronic myelogenous leukemia. By “hit” and grammatical equivalents herein is meant, in the context of the matrix method, that a given peptide is predicted to bind to a given class II MHC allele. In a preferred embodiment, a hit is defined to be a peptide with binding affinity among the top 5%, or 3%, or 1% of binding scores of random peptide sequences. In an alternate embodiment, a hit is defined to be a peptide with a binding affinity that exceeds some threshold, for instance a peptide that is predicted to bind an MHC allele with at least 100 μM or 10 μM or 1 μM affinity. By “immunogenicity” and grammatical equivalents herein is meant the ability of a protein to elicit an immune response, including but not limited to production of neutralizing and non-neutralizing antibodies, formation of immune complexes, complement activation, mast cell activation, inflammation, and anaphylaxis. By “reduced immunogenicity” and grammatical equivalents herein is meant a decreased ability to activate the immune system, when compared to the wild type protein. For example, a variant protein can be said to have “reduced immunogenicity” if it elicits neutralizing or non-neutralizing antibodies in lower titer or in fewer patients than the wild type protein. In a preferred embodiment, the probability of raising neutralizing antibodies is decreased by at least 5%, with at least 50% or 90% decreases being especially preferred. So, if a wild type produces an immune response in 10% of patients, a variant with reduced immunogenicity would produce an immune response in not more than 9.5% of patients, with less than 5% or less than 1% being especially preferred. A variant protein also can be said to have “reduced immunogenicity” if it shows decreased binding to one or more MHC alleles or if it induces T-cell activation in a decreased fraction of patients relative to the parent protein. In a preferred embodiment, the probability of T-cell activation is decreased by at least 5%, with at least 50% or 90% decreases being especially preferred. By “matrix method” and grammatical equivalents thereof herein is meant a method for calculating peptide—MHC affinity in which a matrix is used that contains a score for each possible residue at each position in the peptide, interacting with a given MHC allele. The binding score for a given peptide—MHC interaction is obtained by summing the matrix values for the amino acids observed at each position in the peptide. By “MHC-binding agretopes” and grammatical equivalents herein is meant peptides that are capable of binding to one or more class II MHC alleles with appropriate affinity to enable the formation of MHC—peptide—T-cell receptor complexes and subsequent T-cell activation. MHC-binding agretopes are linear peptide sequences that comprise at least approximately 9 residues. By “parent protein” as used herein is meant a protein that is subsequently modified to generate a variant protein. Said parent protein may be a wild-type or naturally occurring protein, or a variant or engineered version of a naturally occurring protein. “Parent protein” may refer to the protein itself, compositions that comprise the parent protein, or any amino acid sequence that encodes it. Accordingly, by “parent TNF Super Family member protein” as used herein is meant a TNF Super Family member protein that is modified to generate a variant TNF Super Family member protein. By “patient” herein is meant both humans and other animals, particularly mammals, and organisms. Thus the methods are applicable to both human therapy and veterinary applications. In the preferred embodiment the patient is a mammal, and in the most preferred embodiment the patient is human. By “protein” herein is meant at least two covalently attached amino acids, which includes proteins, polypeptides, oligopeptides and peptides. The protein may be made up of naturally occurring amino acids and peptide bonds, or synthetic peptidomimetic structures, i.e., “analogs” such as peptoids [see Simon et al., Proc. Natl. Acad. Sci. U.S.A. 89(20:9367-71 (1992), entirely incorporated by reference], generally depending on the method of synthesis. For example, homo-phenylalanine, citrulline, and noreleucine are considered amino acids for the purposes of the invention. “Amino acid” also includes amino acid residues such as proline and hydroxyproline. Both D- and L-amino acids may be utilized. By “treatment” herein is meant to include therapeutic treatment, as well as prophylactic, or suppressive measures for the disease or disorder. Thus, for example, successful administration of a variant TNF Super Family member protein prior to onset of the disease may result in treatment of the disease. As another example, successful administration of a variant TNF Super Family member protein after clinical manifestation of the disease to combat the symptoms of the disease comprises “treatment” of the disease. “Treatment” also encompasses administration of a variant TNF Super Family member protein after the appearance of the disease in order to eradicate the disease. Successful administration of an agent after onset and after clinical symptoms have developed, with possible abatement of clinical symptoms and perhaps amelioration of the disease, further comprises “treatment” of the disease. Those “in need of treatment” include mammals already having the disease or disorder, as well as those prone to having the disease or disorder, including those in which the disease or disorder is to be prevented. By “variant TNF Super Family member nucleic acids” and grammatical equivalents herein is meant nucleic acids that encode variant TNF Super Family member proteins. Due to the degeneracy of the genetic code, an extremely large number of nucleic acids may be made, all of which encode the variant TNF Super Family member proteins of the present invention, by simply modifying the sequence of one or more codons in a way which does not change the amino acid sequence of the variant TNF Super Family member. By “variant TNF Super Family member proteins” and grammatical equivalents thereof herein is meant non-naturally occurring TNF Super Family member proteins which differ from the wild type or parent TNF Super Family member protein by at least 1 amino acid insertion, deletion, or substitution. TNF Super Family member variants are characterized by the predetermined nature of the variation, a feature that sets them apart from naturally occurring allelic or interspecies variation of the TNF Super Family member protein sequence. The TNF Super Family member variants typically either exhibit biological activity that is comparable to naturally occurring TNF Super Family member or have been specifically engineered to have alternate biological properties. The variant TNF Super Family member proteins may contain insertions, deletions, and/or substitutions at the N-terminus, C-terminus, or internally. In a preferred embodiment, variant TNF Super Family member proteins have at least 1 residue that differs from the naturally occurring TNF Super Family member sequence, with at least 2, 3, 4, or 5 different residues being more preferred. Variant TNF Super Family member proteins may contain further modifications, for instance mutations that alter stability or solubility or which enable or prevent posttranslational modifications such as PEGylation or glycosylation. Variant TNF Super Family member proteins may be subjected to co- or post-translational modifications, including but not limited to synthetic derivatization of one or more side chains or termini, glycosylation, PEGylation, circular permutation, cyclization, fusion to proteins or protein domains, and addition of peptide tags or labels. By “wild type or wt” and grammatical equivalents thereof herein is meant an amino acid sequence or a nucleotide sequence that is found in nature and includes allelic variations; that is, an amino acid sequence or a nucleotide sequence that has not been intentionally modified. In a preferred embodiment, the wild type sequence is SEQ_ID NO:1.

Identification of MHC-Binding Agretopes in TNF Super Family Members

MHC-binding peptides are obtained from proteins by a process called antigen processing. First, the protein is transported into an antigen presenting cell (APC) by endocytosis or phagocytosis. A variety of proteolytic enzymes then cleave the protein into a number of peptides. These peptides can then be loaded onto class II MHC molecules, and the resulting peptide-MHC complexes are transported to the cell surface. Relatively stable peptide-MHC complexes can be recognized by T-cell receptors that are present on the surface of naive T cells. This recognition event is required for the initiation of an immune response. Accordingly, blocking the formation of stable peptide-MHC complexes is an effective approach for preventing unwanted immune responses.

The factors that determine the affinity of peptide-MHC interactions have been characterized using biochemical and structural methods. Peptides bind in an extended conformation bind along a groove in the class II MHC molecule. While peptides that bind class II MHC molecules are typically approximately 13-18 residues long, a nine-residue region is responsible for most of the binding affinity and specificity. The peptide binding groove can be subdivided into “pockets”, commonly named P1 through P9, where each pocket is comprises the set of MHC residues that interacts with a specific residue in the peptide. A number of polymorphic residues face into the peptide-binding groove of the MHC molecule. The identity of the residues lining each of the peptide-binding pockets of each MHC molecule determines its peptide binding specificity. Conversely, the sequence of a peptide determines its affinity for each MHC allele.

Several methods of identifying MHC-binding agretopes in protein sequences are known in the art and may be used to identify agretopes in TNF Super Family members. Sequence-based information can be used to determine a binding score for a given peptide—MHC interaction (see for example Mallios, Bioinformatics 15: 432-439 (1999); Mallios, Bioinformatics 17: p942-948 (2001); Sturniolo et. al. Nature Biotech. 17: 555-561(1999), all entirely incorporated by reference). It is possible to use structure-based methods in which a given peptide is computationally placed in the peptide-binding groove of a given MHC molecule and the interaction energy is determined (for example, see WO 98/59244 and WO 02/069232, entirely incorporated by reference). Such methods may be referred to as “threading” methods. Alternatively, purely experimental methods can be used; for example a set of overlapping peptides derived from the protein of interest can be experimentally tested for the ability to induce T-cell activation and/or other aspects of an immune response. (see for example WO 02/77187, entirely incorporated by reference).

In a preferred embodiment, MHC-binding propensity scores are calculated for each 9-residue frame along the TNF Super Family sequence using a matrix method (see Sturniolo et. al., supra; Marshall et. al., J. Immunol. 154: 5927-5933 (1995), and Hammer et al., J. Exp. Med. 180: 2353-2358 (1994), entirely incorporated by reference). It is also possible to consider scores for only a subset of these residues, or to consider also the identities of the peptide residues before and after the 9-residue frame of interest. The matrix comprises binding scores for specific amino acids interacting with the peptide binding pockets in different human class II MHC molecule. In the most preferred embodiment, the scores in the matrix are obtained from experimental peptide binding studies. In an alternate preferred embodiment, scores for a given amino acid binding to a given pocket are extrapolated from experimentally characterized alleles to additional alleles with identical or similar residues lining that pocket. Matrices that are produced by extrapolation are referred to as “virtual matrices”.

In a preferred embodiment, the matrix method is used to calculate scores for each peptide of interest binding to each allele of interest. Several methods can then be used to determine whether a given peptide will bind with significant affinity to a given MHC allele. In one embodiment, the binding score for the peptide of interest is compared with the binding propensity scores of a large set of reference peptides. Peptides whose binding propensity scores are large compared to the reference peptides are likely to bind MHC and may be classified as “hits”. For example, if the binding propensity score is among the highest 1% of possible binding scores for that allele, it may be scored as a “hit” at the 1% threshold. The total number of hits at one or more threshold values is calculated for each peptide. In some cases, the binding score may directly correspond with a predicted binding affinity. Then, a hit may be defined as a peptide predicted to bind with at least 100 μM or 10 μM or 1 μM affinity.

In a preferred embodiment, the number of hits for each 9-mer frame in the protein is calculated using one or more threshold values ranging from 0.5% to 10%. In an especially preferred embodiment, the number of hits is calculated using 1%, 3%, and 5% thresholds. In a preferred embodiment, MHC-binding agretopes are identified as the 9-mer frames that bind to several class II MHC alleles. In an especially preferred embodiment, MHC-binding agretopes are predicted to bind at least 10 alleles at 5% threshold and/or at least 5 alleles at 1% threshold. Such 9-mer frames may be especially likely to elicit an immune response in many members of the human population. In a preferred embodiment, MHC-binding agretopes are predicted to bind MHC alleles that are present in at least 0.01-10% of the human population. Alternatively, to treat conditions that are linked to specific class II MHC alleles, MHC-binding agretopes are predicted to bind MHC alleles that are present in at least 0.01-10% of the relevant patient population.

Data about the prevalence of different MHC alleles in different ethnic and racial groups has been acquired by groups such as the National Marrow Donor Program (NMDP); for example see Mignot et al. Am. J. Hum. Genet. 68: 686-699 (2001), Southwood et al. J. Immunol. 160: 3363-3373 (1998), Hurley et al. Bone Marrow Transplantation 25: 136-137 (2000), Sintasath Hum. Immunol. 60: 1001 (1999), Collins et al. Tissue Antigens 55: 48 (2000), Tang et al. Hum. Immunol. 63: 221 (2002), Chen et al. Hum. Immunol. 63: 665 (2002), Tang et al. Hum. Immunol. 61: 820 (2000), Gans et al. Tissue Antigens 59: 364-369, and Baldassarre et al. Tissue Antigens 61: 249-252 (2003), all entirely incorporated by reference.

In a preferred embodiment, MHC binding agretopes are predicted for MHC heterodimers comprising highly prevalent MHC alleles. Class II MHC alleles that are present in at least 10% of the US population include but are not limited to: DPA1*0103, DPA1*0201, DPB1*0201, DPB1*0401, DPB1*0402, DQA1*0101, DQA1*0102, DQA1*0201, DQA1*0501, DQB1*0201, DQB1*0202, DQB1*0301, DQB1*0302, DQB1*0501, DQB1*0602, DRA*0101, DRB1*0701, DRB1*1501, DRB1*0301, DRB1*0101, DRB1*1101, DRB1*1301, DRB3*0101, DRB3*0202, DRB4*0101, DRB4*0103, and DRB5*0101.

In a preferred embodiment, MHC binding agretopes are also predicted for MHC heterodimers comprising moderately prevalent MHC alleles. Class II MHC alleles that are present in 1% to 10% of the US population include but are not limited to: DPA1*0104, DPA1*0302, DPA1*0301, DPB1*0101, DPB1*0202, DPB1*0301, DPB1*0501, DPB1*0601, DPB1*0901, DPB1*1001, DPB1*1101, DPB1*1301, DPB1*1401, DPB1*1501, DPB1*1701, DPB1*1901, DPB1*2001, DQA1*0103, DQA1*0104, DQA1*0301, DQA1*0302, DQA1*0401, DQB1*0303, DQB1*0402, DQB1*0502, DQB1*0503, DQB1*0601, DQB1*0603, DRB1*1302, DRB1*0404, DRB1*0801, DRB1*0102, DRB1*1401, DRB1*1104, DRB1*1201, DRB1*1503, DRB1*0901, DRB1*1601, DRB1*0407, DRB1*1001, DRB1*1303, DRB1*0103, DRB1*1502, DRB1*0302, DRB1*0405, DRB1*0402, DRB1*1102, DRB1*0803, DRB1*0408, DRB1*1602, DRB1*0403, DRB3*0301, DRB5*0102, and DRB5*0202.

MHC binding agretopes may also be predicted for MHC heterodimers comprising less prevalent alleles. Information about MHC alleles in humans and other species can be obtained, for example, from the IMGT/HLA sequence database (.ebi.ac.uk/imgt/hla/).

In an especially preferred embodiment, an immunogenicity score is determined for each peptide, wherein said score depends on the fraction of the population with one or more MHC alleles that are hit at multiple thresholds. For example, the equation
Iscore=N(W₁P₁+W₃P₃+W₅P₅)
may be used, where P₁ is the percent of the population hit at 1%, P₃ is the percent of the population hit at 3%, P₅ is the percent of the population hit at 5%, each W is a weighting factor, and N is a normalization factor. In a preferred embodiment, W₁=10, W₃=5, W₅=2, and N is selected so that possible scores range from 0 to 100. In this embodiment, agretopes with Iscore greater than or equal to 10 are preferred and agretopes with Iscore greater than or equal to 25 are especially preferred. Preferred MHC-binding agretopes are those agretopes that are predicted to bind at a 3% threshold to MHC alleles and are present in at least 5% of the population.

In an additional preferred embodiment, MHC-binding agretopes are identified as the 9-mer frames that are located among “nested” agretopes, or overlapping 9-residue frames that are each predicted to bind a significant number of alleles. Such sequences may be especially likely to elicit an immune response. Preferred MHC-binding agretopes are those agretopes that are predicted to bind, at a 3% threshold, to MHC alleles that are present in at least 5% of the population. Especially preferred MHC-binding agretopes are those agretopes that are predicted to bind, at a 1% threshold, to MHC alleles that are present in at least 10% of the population.

Preferred MHC-binding agretopes in BAFF include, but are not limited to, agretope 2: residues 168-176; agretope 3: residues 169-177; agretope 6: residues 192-200; agretope 7: residues 193-201; agretope 9: residues 200-208; agretope 10: residues 212-220; agretope 12: residues 226-234; agretope 14: residues 230-238; agretope 16: residues 276-284. Especially preferred MHC-binding agretopes in BAFF include, but are not limited to, agretope 2: residues 168-176; agretope 6: residues 192-200; agretope 9: residues 200-208; agretope 10: residues 212-220; agretope 12: residues 226-234; agretope 16: residues 276-284.

Preferred MHC-binding agretopes in RANKL include, but are not limited to, agretope 2: residues 207-215; agretope 3: residues 213-221; agretope 4: residues 214-222; agretope 5: residues 215-223; agretope 6: residues 222-230; agretope 9: residues 238-246; agretope 10: residues 239-247; agretope 12: residues 241-249; agretope 14: residues 270-278; agretope 15: residues 277-285; agretope 17: residues 289-297; agretope 18: residues 308-316. Especially preferred MHC-binding agretopes in RANKL include, but are not limited to, agretope 3: residues 213-221; agretope 4: residues 214-222; agretope 10: residues 239-247; agretope 12: residues 241-249; agretope 15: residues 277-285; agretope 17: residues 289-297.

Preferred MHC-binding agretopes in APRIL include, but are not limited to, agretope 1: residues 117-125; agretope 2: residues 120-128; agretope 4: residues 138-146; agretope 5: residues 142-150; agretope 6: residues 155-163; agretope 7: residues 162-170; agretope 9: residues 164-172; agretope 10: residues 170-178; agretope 11: residues 194-202; agretope 12: residues 197-205; agretope 15: residues 228-236. Especially preferred MHC-binding agretopes in APRIL include, but are not limited to, agretope 5: residues 142-150; agretope 9: residues 164-172; agretope 10: residues 170-178; agretope 11: residues 194-202.

Preferred MHC-binding agretopes in CD40L include, but are not limited to, agretope 1: residues 145-153; agretope 2: residues 146-154; agretope 3: residues 152-160; agretope 4: residues 168-176; agretope 5: residues 169-177; agretope 6: residues 170-178; agretope 7: residues 171-179; agretope 9: residues 189-197; agretope 10: residues 204-212; agretope 11: residues 205-213; agretope 12: residues 206-214; agretope 13: residues 223-231; agretope 14: residues 229-237; agretope 15: residues 237-245. Especially preferred MHC-binding agretopes in CD40L include, but are not limited to, agretope 12: residues 206-214.

Preferred MHC-binding agretopes in TRAIL include, but are not limited to, agretope 1: residues 151-159; agretope 2: residues 174-182; agretope 3: residues 181-189; agretope 6: residues 206-214; agretope 7: residues 207-215; agretope 9: residues 220-228; agretope 10: residues 221-229; agretope 12: residues 237-245; agretope 14: residues 256-264; agretope 15: residues 257-265. Especially preferred MHC-binding agretopes in TRAIL include, but are not limited to, agretope 2: residues 174-182; agretope 7: residues 207-215; agretope 10: residues 221-229; agretope 14: residues 256-264; agretope 15: residues 257-265.

Confirmation of MHC-Binding Agretopes

In a preferred embodiment, the immunogenicity of the above-predicted MHC-binding agretopes is experimentally confirmed by measuring the extent to which peptides comprising each predicted agretope can elicit an immune response. However, it is possible to proceed from agretope prediction to agretope removal without the intermediate step of agretope confirmation.

Several methods, discussed in more detail below, can be used for experimental confirmation of agretopes. For example, sets of naïve T cells and antigen presenting cells from matched donors can be stimulated with a peptide containing an agretope of interest, and T-cell activation can be monitored. It is also possible to first stimulate T cells with the whole protein of interest, and then re-stimulate with peptides derived from the whole protein. If sera are available from patients who have raised an immune response to TNF Super Family, it is possible to detect mature T cells that respond to specific epitopes. In a preferred embodiment, interferon gamma or IL-5 production by activated T-cells is monitored using Elispot assays, although it is also possible to use other indicators of T-cell activation or proliferation such as tritiated thymidine incorporation or production of other cytokines.

Patient Genotype Analysis and Screening

HLA genotype is a major determinant of susceptibility to specific autoimmune diseases (see for example Nepom Clin. Immunol. Immunopathol. 67: S50-S55 (1993), entirely incorporated by reference) and infections (see for example Singh et. al. Emerg. Infect. Dis. 3: 41-49 (1997), entirely incorporated by reference). Furthermore, the set of MHC alleles present in an individual can affect the efficacy of some vaccines (see for example Cailat-Zucman et. al. Kidney Int. 53: 1626-1630 (1998) and Poland et. al. Vaccine 20: 430-438 (2001), both entirely incorporated by reference). HLA genotype may also confer susceptibility for an individual to elicit an unwanted immune response to a TNF Super Family therapeutic.

In a preferred embodiment, class II MHC alleles that are associated with increased or decreased susceptibility to elicit an immune response to TNF Super Family proteins are identified. For example, patients treated with TNF Super Family therapeutics may be tested for the presence of anti-TNF Super Family antibodies and genotyped for class II MHC. Alternatively, T-cell activation assays such as those described above may be conducted using cells derived from a number of genotyped donors. Alleles that confer susceptibility to TNF Super Family immunogenicity may be defined as those alleles that are significantly more common in those who elicit an immune response versus those who do not. Similarly, alleles that confer resistance to TNF Super Family immunogenicity may be defined as those that are significantly less common in those who do not elicit an immune response versus those that do. It is also possible to use purely computational techniques to identify which alleles are likely to recognize TNF Super Family therapeutics. In one embodiment, the genotype association data is used to identify patients who are especially likely or especially unlikely to raise an immune response to a TNF Super Family therapeutic.

Design of Active, Less-Immunogenic Variants

In a preferred embodiment, the above-determined MHC-binding agretopes are replaced with alternate amino acid sequences to generate active variant TNF Super Family proteins with reduced or eliminated immunogenicity. Alternatively, the MHC-binding agretopes are modified to introduce one or more sites that are susceptible to cleavage during protein processing. If the agretope is cleaved before it binds to a MHC molecule, it will be unable to promote an immune response. There are several possible strategies for integrating methods for identifying less immunogenic sequences with methods for identifying structured and active sequences, including but not limited to those presented below.

In one embodiment, for one or more 9-mer agretope identified above, one or more possible alternate 9-mer sequences are analyzed for immunogenicity as well as structural and functional compatibility. The preferred alternate 9-mer sequences are then defined as those sequences that have low predicted immunogenicity and a high probability of being structured and active. It is possible to consider only the subset of 9-mer sequences that are most likely to comprise structured, active, less immunogenic variants. For example, it may be unnecessary to consider sequences that comprise highly non-conservative mutations or mutations that increase predicted immunogenicity.

In a preferred embodiment, less immunogenic variants of each agretope are predicted to bind MHC alleles in a smaller fraction of the population than the wild type agretope. In an especially preferred embodiment, the less immunogenic variant of each agretope is predicted to bind to MHC alleles that are present in not more than 5% of the population, with not more than 1% or 0.1% being most preferred.

Substitution Matrices

In another especially preferred embodiment, substitution matrices or other knowledge-based scoring methods are used to identify alternate sequences that are likely to retain the structure and function of the wild type protein. Such scoring methods can be used to quantify how conservative a given substitution or set of substitutions is. In most cases, conservative mutations do not significantly disrupt the structure and function of proteins (see for example, Bowie et. al. Science 247: 1306-1310 (1990), Bowie and Sauer Proc. Nat. Acad. Sci. USA 86: 2152-2156 (1989), and Reidhaar-Olson and Sauer Proteins 7: 306-316 (1990), entirely incorporated by reference). However, non-conservative mutations can destabilize protein structure and reduce activity (see for example, Lim et. al. Biochem. 31: 4324-4333 (1992)). Substitution matrices including but not limited to BLOSUM62 provide a quantitative measure of the compatibility between a sequence and a target structure, which can be used to predict non-disruptive substitution mutations (see Topham et al. Prot. Eng. 10: 7-21 (1997), entirely incorporated by reference). The use of substitution matrices to design peptides with improved properties has been disclosed; see Adenot et al. J. Mol. Graph. Model. 17: 292-309 (1999), entirely incorporated by reference.

Substitution matrices include, but are not limited to, the BLOSUM matrices (Henikoff and Henikoff, Proc. Nat. Acad. Sci. USA 89: 10917 (1992), entirely incorporated by reference, the PAM matrices, the Dayhoff matrix, and the like. For a review of substitution matrices, see for example Henikoff Curr. Opin. Struct. Biol. 6: 353-360 (1996), entirely incorporated by reference. It is also possible to construct a substitution matrix based on an alignment of a given protein of interest and its homologs; see for example Henikoff and Henikoff Comput. Appl. Biosci. 12: 135-143 (1996), entirely incorporated by reference. In a preferred embodiment, each of the substitution mutations that are considered has a BLOSUM 62 score of zero or higher. According to this metric, preferred substitutions include, but are not limited to:

TABLE 1
Conservative mutations

| Wild type | Preferred |
|---|---|
| residue | substitutions |
| A | C S T A G V |
| C | C A |
| D | S N D E Q |
| E | S N D E Q H R K |
| F | M I L F Y W |
| G | S A G N |
| H | N E Q H R Y |
| I | M I L V F |
| K | S N E Q R K |
| L | M I L V F |
| M | Q M I L V F |
| N | S T G N D E Q H R K |
| P | P |
| Q | S N D E Q H R K M |
| R | N E Q H R K |
| S | S T A G N D E Q K |
| T | T A M I L V |
| V | S T A N V |
| W | F Y W |
| Y | H F Y W |

In addition, it is preferred that the total BLOSUM 62 score of an alternate sequence for a nine residue MHC-binding agretope is decreased only modestly when compared to the BLOSUM 62 score of the wild type nine residue agretope. In a preferred embodiment, the score of the variant 9-mer is at least 50% of the wild type score, with at least 67%, 75%, 80% or 90% being more preferred.

Alternatively, alternate sequences can be selected that minimize the absolute reduction in BLOSUM score; for example it is preferred that the score decrease for each 9-mer is less than 20, with score decreases of less than about 10 or about 5 being especially preferred. The exact value may be chosen to produce a library of alternate sequences that is experimentally tractable and also sufficiently diverse to encompass a number of active, stable, less immunogenic variants.

In a preferred embodiment, substitution mutations are preferentially introduced at positions that are substantially solvent exposed. As is known in the art, solvent exposed positions are typically more tolerant of mutation than positions that are located in the core of the protein.

In another preferred embodiment, substitution mutations are preferentially introduced at positions that are not highly conserved. As is known in the art, positions that are highly conserved among members of a protein family are often important for protein function, stability, or structure, while positions that are not highly conserved often may be modified without significantly impacting the structural or functional properties of the protein.

Alanine Substitutions

In an alternate embodiment, one or more alanine substitutions may be made, regardless of whether an alanine substitution is conservative or non-conservative. As is known in the art, incorporation of sufficient alanine substitutions may be used to disrupt intermolecular interactions.

In a preferred embodiment, variant 9-mers are selected such that residues that have been or can be identified as especially critical for maintaining the structure or function of TNF Super Family retain their wild type identity. In alternate embodiments, it may be desirable to produce variant TNF Super Family proteins that do not retain wild type activity. In such cases, residues that have been identified as critical for function may be specifically targeted for modification.

Positions that mediate binding to the receptors BAFF-R, TACI, and BCMA include, but are not limited to, Q159, Y163, D203, T205, Y206, A207, L211, R231, 1233, P264, R265, and D275, more preferably D203, T205, Y206, 1233, R265, and D275. Residues that may impact the oligomer subunit exchange properties of BAFF include, but are not limited to, T205, Y206, F220, E223, V227, T228, I233, L240, D273 and D275.

RANKL contacts its receptor, RANK, and its decoy receptor, OPG, through three dimensional epitopes located in the Large Domain, the Small Domain, and the DE loop. Modifications to the receptor contact positions are expected to have direct effects on receptor binding or signaling. Positions that contact receptor include, but are not limited to, the Large Domain positions 172, 187-193, 222-228, 267-270, 297, and 300-302; the Small Domain positions 179-183 and 233-241; and the DE Loop positions 246-253 and 284.

RANKL is active as a trimer. Accordingly, modifications to the trimer interface positions are expected to have direct effects on RANKL activity. The trimer Interface includes positions 163, 165, 167, 193, 195, 213, 215, 217, 219, 221, 235, 237, 239, 244, 253-264, 268, 271-282, 300, 302, 304-305, 307, 311, and 313-314.

Homology modeling with APRIL's closest homolog (BAFF) and sequence alignment with homologous TNF ligands can be used to predict positions important for structure, receptor binding and activity (Karpusas et al. J Mol Biol 315: 1145-1154 (2002)). A polymorphism in the APRIL gene resulting in amino acid substitution G67R is associated with SLE (Koyama et al. Rheumatology (Oxford) 42: 980-985 (2003), entirely incorporated by reference).

Furthermore, a number of residues may be targeted for mutagenesis in order to yield a APRIL variant that functions as an antagonist, receptor specific agonist, or superagonist. Suitable residues include but are not limited to Large Domain receptor contact residues (positions 121, 139-142, 170-174, 205-208, and 237-241), Small Domain receptor contact residues (positions 175-181 and 195-197), and DE loop receptor contact residues (positions 186-190). In addition, trimer interface positions may be modified, for example to promote trimer exchange or to stabilize desired trimeric structures. Trimer interface positions include but are not limited to residues 115, 117, 119, 142, 144, 162, 164, 166, 168, 170, 176, 177, 192, 194, 201, 208-216, 237, 239, 241, 242, 245, 248, 250, and 251. Especially preferred trimer interface positions are APRIL positions 142, 144, 162, 164, 216 and 251.

Mutagenesis studies indicate that CD40L residues K143, Y145, Y146, R203, R207, and Q220 are important for CD40 receptor binding and/or activity (Bajorath et al. Biochemistry 34: 1833-1844 (1995), Bajorath et al. Biochemistry 34: 9884-9892 (1995), Singh et al. Protein Sci 7: 1124-1135 (1998)), entirely incorporated by reference. CD40L mutations associated with the X-linked form of hyper-IgM syndrome disrupt the normal function of CD40L; these mutations include A123E, H125R, V126D, V126A, W140C, W140G, W140R, W140X, G144E, T147N, L155P, Y170P, A173D, Q174R, T1761, A183@, S184X, Q186X, L193@, L195P, R200X, E202X, A208D, C218X, Q220X, Q221X, H224Y, G226A, G227V, L231S, Q232X, A235P, S236X, V237E, T254M, G257D, G257S, L258S, where X denotes a deletion of the amino acid and @ denotes insertions of one or more amino acids at these locations (uta.fi/imt/bioinfo/CD40Lbase).

Furthermore, a number of residues may be targeted for mutagenesis in order to yield a CD40L variant that functions as an antagonist of wild type CD40L protein or as a superagonist of CD40. Suitable residues include but are not limited to Large Domain receptor contact residues (positions 28-34, 63-69, 112-115, and 137-14), Small Domain receptor contact residues (positions 72-79 and 95-98), and DE loop receptor contact residues (positions 84-89). In addition, trimer interface positions may be modified, for example to promote trimer exchange or to stabilize desired trimeric structures. Trimer interface positions include but are not limited to residues 11, 13, 15, 34, 36, 53-55, 57, 59, 61, 63, 72, 73, 75, 77, 119, 87, 91-99, 102-104, 109, 112-125, 147-149, 151, and 155-157. Especially preferred trimer interface positions to be modified are positions 57, 34, and 91.

Alanine scanning mutagenesis of TRAIL reveals two clusters of residues essential for receptor binding and biological activity; these are located along the walls of a surface crevice formed by adjoining monomers that runs from the wider part of the trimer to the variable loops at the tip. Substitutions at Tyr216 at the top and Gln 205 at the tip each decreased apoptotic activity more than 300-fold, while substitutions at Val207, Glu236, or Tyr237 decreased activity more than 5-fold; all but one of these point mutants showed at least a 5-fold decreased affinity for DR4, DR5, and DcR2. Mutants D218A and D269A slightly increased apoptotic activity, but did not affect receptor binding. A zinc atom coordinated by three symmetry-related Cys230 residues in the trimerization interface appears essential for trimer stability and optimal biological activity; mutation of Cys230 to alanine or serine results in 20- and 70-fold reductions in apoptotic activity, respectively, decreases receptor binding by at least 200-fold, and reduces the stability of the trimeric structure. Removal of zinc from wild type TRAIL by dialysis with chelating agents results in a significant decrease in receptor binding affinity and a 90-fold reduction in apoptotic activity; zinc depleted TRAIL forms poorly active, disulfide-linked dimers. See Bodmer et al. J Biol Chem 275: 20632-20637 (2000), Hymowitz et al. Biochemistry 39: 633-640 (2000), entirely incorporated by reference.

Based on a model of the TRAIL-sDR4 complex, deletion of the AN″ insertion loop (TRAIL residues 137-152) and point mutants of residues believed to interact with the receptor (El44N/K on the β turn of the AA″ loop, D218N/K on the DE loop and D267N/K on the GH loop) resulted in decreased or no cytotoxic activity using a Jurkat T cell assay. Decreased cytotoxic activity or sDR5 binding was also obtained with other TRAIL variants containing deletions in the AA″ loop (residues 132-135, Ser-Leu-Leu sequence instead of residues 135-153). It has therefore been suggested that the frame insertion of 12-16 amino acids in the M″ loop, unique to TRAIL among TNF family ligands, is critical in providing the conformational flexibility required for translocation of the M″ loop to the central binding interface upon complex formation, and may be important in conferring receptor recognition specificity. See Cha et al. Immunity 11: 253-261 (1999), Mongkolsapaya et al. Nat Struct Biol 6: 1048-1053 (1999), Cha et al. J Biol Chem 275: 31171-31177 (2000), entirely incorporated by reference.

Leucine zippers introduced to the N-terminus to facilitate multimerization result in mutants (LZ-TRAIL) that are superior to normal and cross-linked TRAIL in causing cell lysis in human and mouse cell lines; LZ-TRAIL also confers survival to tumor challenge in mice without hepatotoxicity. See Walczak et al. Nat Med 5: 157-163 (1999), entirely incorporated by reference. It has been suggested that substitution of Asn228 with a large hydrophobic residue could induce stronger intersubunit interactions with Tyr240 and improve stability.

Protein Design Methods

Protein design methods and MHC agretope identification methods may be used together to identify stable, active, and minimally immunogenic protein sequences (see WO03/006154, entirely incorporated by reference). The combination of approaches provides significant advantages over the prior art for immunogenicity reduction, as most of the reduced immunogenicity sequences identified using other techniques fail to retain sufficient activity and stability to serve as therapeutics.

Protein design methods may identify non-conservative or unexpected mutations that nonetheless confer desired functional properties and reduced immunogenicity, as well as identifying conservative mutations. Nonconservative mutations are defined herein to be all substitutions not included in Table 1 above; nonconservative mutations also include mutations that are unexpected in a given structural context, such as mutations to hydrophobic residues at the protein surface and mutations to polar residues in the protein core.

Furthermore, protein design methods may identify compensatory mutations. For example, if a given first mutation that is introduced to reduce immunogenicity also decreases stability or activity, protein design methods may be used to find one or more additional mutations that serve to recover stability and activity while retaining reduced immunogenicity. Similarly, protein design methods may identify sets of two or more mutations that together confer reduced immunogenicity and retained activity and stability, even in cases where one or more of the mutations, in isolation, fails to confer desired properties.

A wide variety of methods are known for generating and evaluating sequences. These include, but are not limited to, sequence profiling (Bowie and Eisenberg, Science 253(5016): 164-70, (1991)), residue pair potentials (Jones, Protein Science 3: 567-574, (1994)), and rotamer library selections (Dahiyat and Mayo, Protein Sci 5(5): 895-903 (1996); Dahiyat and Mayo, Science 278(5335): 82-7 (1997); Desjarlais and Handel, Protein Science 4: 2006-2018 (1995); Harbury et al, PNAS USA 92(18): 8408-8412 (1995); Kono et al., Proteins: Structure, Function and Genetics 19: 244-255 (1994); Hellinga and Richards, PNAS USA 91: 5803-5807 (1994), entirely incorporated by reference).

Protein Design Automation® (PDA®) Technology

In an especially preferred embodiment, rational design of improved TNF Super Family variants is achieved by using Protein Design Automation® (PDA®) technology. (See U.S. Pat. Nos. 6,188,965; 6,269,312; 6,403,312; WO98/47089 and U.S. Ser. Nos. 09/058,459, 09/127,926, 60/104,612, 60/158,700, Ser. No. 09/419,351, 60/181,630, 60/186,904, Ser. Nos. 09/419,351, 09/782,004 and 09/927,790, 60/347,772, and Ser. No. 10/218,102; and PCT/US01/218,102 and U.S. Ser. No. 10/218,102, U.S. Ser. No. 60/345,805; U.S. Ser. No. 60/373,453 and U.S. Ser. No. 60/374,035, all entirely incorporated by reference.)

PDA® technology couples computational design algorithms that generate quality sequence diversity with experimental high-throughput screening to discover proteins with improved properties. PDA® utilizes three-dimensional structural information. The computational component uses atomic level scoring functions, side chain rotamer sampling, and advanced optimization methods to accurately capture the relationships between protein sequence, structure, and function. Calculations begin with the three-dimensional structure of the protein and a strategy to optimize one or more properties of the protein. PDA® technology then explores the sequence space comprising all pertinent amino acids (including unnatural amino acids, if desired) at the positions targeted for design. This is accomplished by sampling conformational states of allowed amino acids and scoring them using a parameterized and experimentally validated function that describes the physical and chemical forces governing protein structure. Powerful combinatorial search algorithms are then used to search through the initial sequence space, which may constitute 10⁵⁰ sequences or more, and quickly return a tractable number of sequences that are predicted to satisfy the design criteria. Useful modes of the technology span from combinatorial sequence design to prioritized selection of optimal single site substitutions. PDA® technology has been applied to numerous systems including important pharmaceutical and industrial proteins and has a demonstrated record of success in protein optimization.

In a most preferred embodiment, the structure of a TNF Super Family member is determined using X-ray crystallography or NMR methods, which are well known in the art. Crystal structures of some human TNF Super Family members have been solved to high resolution: human BAFF (PDB code 1KXG; Oren et al. 2002 Nat. Struct. Biol. 9: 288), human TRAIL (PDB code 1D4V; Mongkolsapaya et al. 1999 Nat. Struct. Biol. 6:1043), human CD40L (PDB code 1ALY; Karpusas et al. 1995 Structure 3:1426), all entirely incorporated by reference. Using homology modeling methods known in the art, the structures of human RANKL and APRIL were determined using the sequences of human RANKL and human APRIL and the structures of murine RANKL (PDB code 1/QA; Ito et al. 2002 J. Biol. Chem. 277: 6631) and murine APRIL (PDB code 1XU2; Hymowitz et al. 2005 J. Biol. Chem. 280:7218), both entirely incorporated by reference. Furthermore, crystal structures of the BAFF/BAFF-R complex (PDB codes 1OTZ and 1P0T; Kim et. al. 2003 Nat. Struct. Biol. 10:342), the BAFF/BCMA complex (PDB code 1OQD; Liu et. al. 2003 Nature 423:49), the TRAIL/Death Receptor 5 complex (PDB code 1D0G; Hymowitz et al. 1999 Mol. Cell 4:563), the APRIL/TACI complex (PDB code 1XU1; Hymowitz et al. 2005 J. Biol. Chem. 280:7218), and the APRIL/BCMA complex (PDB code 1Xu2; Hymowitz et al. 2005 J. Biol. Chem. 280:7218) have been determined, all entirely incorporated by reference.

In a preferred embodiment, the results of matrix method calculations are used to identify which of the 9 amino acid positions within the agretope(s) contribute most to the overall binding propensities for each particular allele “hit”. This analysis considers which positions (P1-P9) are occupied by amino acids which consistently make a significant contribution to MHC binding affinity for the alleles scoring above the threshold values. Matrix method calculations are then used to identify amino acid substitutions at said positions that would decrease or eliminate predicted immunogenicity and PDA® technology is used to determine which of the alternate sequences with reduced or eliminated immunogenicity are compatible with maintaining the structure and function of the protein.

In an alternate preferred embodiment, the residues in each agretope are first analyzed by one skilled in the art to identify alternate residues that are potentially compatible with maintaining the structure and function of the protein. Then, the set of resulting sequences are computationally screened to identify the least immunogenic variants. Finally, each of the less immunogenic sequences are analyzed more thoroughly in PDA® technology protein design calculations to identify protein sequences that maintain the protein structure and function and decrease immunogenicity.

In an alternate preferred embodiment, each residue that contributes significantly to the MHC binding affinity of an agretope is analyzed to identify a subset of amino acid substitutions that are potentially compatible with maintaining the structure and function of the protein. This step may be performed in several ways, including PDA® calculations or visual inspection by one skilled in the art. Sequences may be generated that contain all possible combinations of amino acids that were selected for consideration at each position. Matrix method calculations can be used to determine the immunogenicity of each sequence. The results can be analyzed to identify sequences that have significantly decreased immunogenicity. Additional PDA® calculations may be performed to determine which of the minimally immunogenic sequences are compatible with maintaining the structure and function of the protein.

In an alternate preferred embodiment, pseudo-energy terms derived from the peptide binding propensity matrices are incorporated directly into the PDA® technology calculations. In this way, it is possible to select sequences that are active and less immunogenic in a single computational step.

Combining Immunogenicity Reduction Strategies

In a preferred embodiment, more than one method is used to generate variant proteins with desired functional and immunological properties. For example, substitution matrices may be used in combination with PDA® technology calculations. Strategies for immunogenicity reduction include, but are not limited to, those described in U.S. Ser. No. 11/004,590, filed Dec. 3, 2004, entirely incorporated by reference.

In a preferred embodiment, a variant protein with reduced binding affinity for one or more class II MHC alleles is further engineered to confer improved solubility. As protein aggregation may contribute to unwanted immune responses, increasing protein solubility may reduce immunogenicity (see for example SIFN).

In an additional preferred embodiment, a variant protein with reduced binding affinity for one or more class II MHC alleles is further modified by derivitization with PEG or another molecule. As is known in the art, PEG may sterically interfere with antibody binding or improve protein solubility, thereby reducing immunogenicity. In an especially preferred embodiment, rational PEGylation methods are used U.S. Ser. No. 10/956,352, filed Sep. 30, 2004, entirely incorporated by reference. In a preferred embodiment, PDA® technology and matrix method calculations are used to remove more than one MHC-binding agretope from a protein of interest.

Generating the Variants

Variant TNF Super Family proteins of the invention and nucleic acids encoding them may be produced using a number of methods known in the art. In a preferred embodiment, nucleic acids encoding the TNF Super Family variants are prepared by total gene synthesis, or by site-directed mutagenesis of a nucleic acid encoding a parent TNF Super Family protein. Methods including template-directed ligation, recursive PCR, cassette mutagenesis, site-directed mutagenesis or other techniques that are well known in the art may be utilized (see for example Strizhov et al. PNAS 93:15012-15017 (1996), Prodromou and Perl, Prot. Eng. 5: 827-829 (1992), Jayaraman and Puccini, Biotechniques 12: 392-398 (1992), and Chalmers et al. Biotechniques 30: 249-252 (2001)), entirely incorporated by reference.

In a preferred embodiment, TNF Super Family variants are cloned into an appropriate expression vector and expressed in E. coli (see McDonald, J. R., Ko, C., Mismer, D., Smith, D. J. and Collins, F. Biochim. Biophys. Acta 1090: 70-80 (1991), entirely incorporated by reference). In an alternate preferred embodiment, TNF Super Family variants are expressed in mammalian cells, yeast, baculovirus, or in vitro expression systems. A number of expression systems and methods for their use are well known in the art (see Current Protocols in Molecular Biology, Wiley & Sons, and Molecular Cloning—A Laboratory Manual—3rd Ed., Cold Spring Harbor Laboratory Press, New York (2001), entirely incorporated by reference). The choice of codons, suitable expression vectors and suitable host cells will vary depending on a number of factors, and may be easily optimized as needed.

In a preferred embodiment, the TNF Super Family variants are purified or isolated after expression. Standard purification methods include electrophoretic, molecular, immunological and chromatographic techniques, including ion exchange, hydrophobic, affinity, and reverse-phase HPLC chromatography, and chromatofocusing. For example, a TNF Super Family variant may be purified using a standard anti-recombinant protein antibody column. Ultrafiltration and diafiltration techniques, in conjunction with protein concentration, are also useful. For general guidance in suitable purification techniques, see Scopes, R., Protein Purification, Springer-Verlag, NY, 3rd ed. (1994), entirely incorporated by reference. The degree of purification necessary will vary depending on the desired use, and in some instances no purification will be necessary.

Assaying the Activity of the Variants

The variant TNF Super Family proteins of the invention may be tested for activity using any of a number of methods, including but not limited to those described below. Suitable binding assays may be used. The kinetic association rate (Kₒₙ) and dissociation rate (K_{off}), and the equilibrium binding constants (K_{d}) may be determined using surface plasmon resonance on a BIAcore instrument following the standard procedure in the literature [Pearce et al., Biochemistry 38:81-89 (1999), entirely incorporated by reference]. Binding affinity and kinetics may also be characterized using proximity assays such as AlphaScreen™ (Packard BioScience®) or microcalorimetry (Isothermal Titration Calorimetry, Differential Scanning Calorimetry), entirely incorporated by reference. Cell-based activity assays include but are not limited to, NF-kB nuclear translocation (Wei et al., Endocrinology 142, 1290-1295, (2001)) or c-Jun (Srivastava et al., JBC 276, 8836-8840 (2001), entirely incorporated by reference) transcription factor activation assays, B-cell proliferation assays, and IgE secretion assays.

Determining the Immunogenicity of the Variants

In a preferred embodiment, the immunogenicity of the TNF Super Family variants is determined experimentally to confirm that the variants do have reduced or eliminated immunogenicity relative to the parent protein. In a preferred embodiment, ex vivo T-cell activation assays are used to experimentally quantitate immunogenicity. In this method, antigen presenting cells and naive T cells from matched donors are challenged with a peptide or whole protein of interest one or more times. Then, T cell activation can be detected using a number of methods, for example by monitoring production of cytokines or measuring uptake of tritiated thymidine. In the most preferred embodiment, interferon gamma production is monitored using Elispot assays (see Schmittel et. al. J. Immunol. Meth., 24: 17-24 (2000), entirely incorporated by reference). Other suitable T-cell assays include those disclosed in Meidenbauer, et al. Prostate 43, 88-100 (2000); Schultes, B. C and Whiteside, T. L., J. Immunol. Methods 279, 1-15 (2003); and Stickler, et al., J. Immunotherapy, 23, 654-660 (2000), all entirely incorporated by reference.

In a preferred embodiment, the PBMC donors used for the above-described T-cell activation assays will comprise class II MHC alleles that are common in patients requiring treatment for TNF Super Family responsive disorders. For example, for most diseases and disorders, it is desirable to test donors comprising all of the alleles that are prevalent in the population. However, for diseases or disorders that are linked with specific MHC alleles, it may be more appropriate to focus screening on alleles that confer susceptibility to TNF Super Family responsive disorders. In a preferred embodiment, the MHC haplotype of PBMC donors or patients that raise an immune response to the wild type or variant TNF Super Family are compared with the MHC haplotype of patients who do not raise a response. This data may be used to guide preclinical and clinical studies as well as aiding in identification of patients who will be especially likely to respond favorably or unfavorably to the TNF Super Family therapeutic.

In an alternate preferred embodiment, immunogenicity is measured in transgenic mouse systems. For example, mice expressing fully or partially human class II MHC molecules may be used. In an alternate embodiment, immunogenicity is tested by administering the TNF Super Family variants to one or more animals, including rodents and primates, and monitoring for antibody formation. Non-human primates with defined MHC haplotypes may be especially useful, as the sequences and hence peptide binding specificities of the MHC molecules in non-human primates may be very similar to the sequences and peptide binding specificities of humans. Similarly, genetically engineered mouse models expressing human MHC peptide-binding domains may be used (see for example Sonderstrup et. al. Immunol. Rev. 172: 335-343 (1999) and Forsthuber et. al. J. Immunol. 167: 119-125 (2001), entirely incorporated by reference).

Formulation and Administration to Patients

Once made, the variant TNF Super Family proteins and nucleic acids of the invention find use in a number of applications. In a preferred embodiment, the variant TNF Super Family proteins are administered to a patient to treat a TNF Super Family responsive disorder. Administration may be therapeutic or prophylactic.

The pharmaceutical compositions of the present invention comprise a variant TNF Super Family protein in a form suitable for administration to a patient. In a preferred embodiment, the pharmaceutical compositions are in a water soluble form, such as being present as pharmaceutically acceptable salts, which is meant to include both acid and base addition salts. “Pharmaceutically acceptable acid addition salt” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid and the like, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. “Pharmaceutically acceptable base addition salts” include those derived from inorganic bases such as sodium, potassium, lithium, ammonium, calcium, magnesium, iron, zinc, copper, manganese, aluminum salts and the like. Particularly preferred are the ammonium, potassium, sodium, calcium, and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, and ethanolamine.

The pharmaceutical compositions may also include one or more of the following: carrier proteins such as serum albumin; buffers such as NaOAc; fillers such as microcrystalline cellulose, lactose, corn and other starches; binding agents; sweeteners and other flavoring agents; coloring agents; and polyethylene glycol. Additives are well known in the art, and are used in a variety of formulations. Combinations of pharmaceutical compositions may be administered. Moreover, the compositions may be administered in combination with other therapeutics.

The administration of the variant TNF Super Family proteins of the present invention, preferably in the form of a sterile aqueous solution, may be done in a variety of ways, including, but not limited to, orally, subcutaneously, intravenously, intranasally, transdermally, intraperitoneally, intramuscularly, parenterally, intrapulmonary, vaginally, rectally, or intraocularly. In some instances, for example, the variant TNF Super Family protein may be directly applied as a solution or spray. Depending upon the manner of introduction, the pharmaceutical composition may be formulated in a variety of ways. In a preferred embodiment, a therapeutically effective dose of a variant TNF Super Family protein is administered to a patient in need of treatment. By “therapeutically effective dose” herein is meant a dose that produces the effects for which it is administered. The exact dose will depend on the purpose of the treatment, and will be ascertainable by one skilled in the art using known techniques. In a preferred embodiment, the concentration of the therapeutically active variant TNF Super Family protein in the formulation may vary from about 0.1 to about 100 weight %. In another preferred embodiment, the concentration of the variant TNF Super Family protein is in the range of 0.003 to 1.0 molar, with dosages from 0.03, 0.05, 0.1, 0.2, and 0.3 millimoles per kilogram of body weight being preferred. As is known in the art, adjustments for variant TNF Super Family protein degradation, systemic versus localized delivery, and rate of new protease synthesis, as well as the age, body weight, general health, sex, diet, time of administration, drug interaction and the severity of the condition may be necessary, and will be ascertainable with routine experimentation by those skilled in the art.

In an alternate embodiment, variant TNF Super Family nucleic acids may be administered; i.e., “gene therapy” approaches may be used. In this embodiment, variant TNF Super Family nucleic acids are introduced into cells in a patient in order to achieve in vivo synthesis of a therapeutically effective amount of variant TNF Super Family protein. Variant TNF Super Family nucleic acids may be introduced using a number of techniques, including but not limited to transfection with liposomes, viral (typically retroviral) vectors, and viral coat protein-liposome mediated transfection [Dzau et al., Trends in Biotechnology 11:205-210 (1993), entirely incorporated by reference]. In some situations it is desirable to provide the nucleic acid source with an agent that targets the target cells, such as an antibody specific for a cell surface membrane protein or the target cell, a ligand for a receptor on the target cell, etc. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262:4429-4432 (1987); and Wagner et al., Proc. Natl. Acad. Sci. U.S.A. 87:3410-3414 (1990), entirely incorporated by reference. For review of gene marking and gene therapy protocols see Anderson et al., Science 256:808-813 (1992), entirely incorporated by reference.

### EXAMPLES

### Example 1

### Identification of MHC-Binding Agretopes in TNF SF Members

Matrix method calculations (Sturniolo, supra) were conducted using the parent TNF SF members sequences: BAFF (SEQ_ID_NO:1); RANKL (SEQ_ID_NO:2); and APRIL (SEQ_ID_NO:3).

Agretopes were predicted for the following alleles, each of which is present in at least 1% of the US population: DRB1*0101, DRB1*0102, DRB1*0301, DRB1*0401, DRB1*0402, DRB1*0404, DRB1*0405, DRB1*0408, DRB1*0701, DRB1*0801, DRB1*1101, DRB1*1102, DRB1*1104, DRB1*1301, DRB1*1302, DRB1*1501, and DRB1*1502.

Table 2. Predicted MHC-binding agretopes in TNF SF members. Iscore, the number of alleles, and the percent of the population hit at 1%, 3%, and 5% thresholds are shown. Especially preferred agretopes are predicted to affect at least 10% of the population, using a 1% threshold.

TABLE 2.A
Predicted MHC-binding agretopes in BAFF.

| Agretope | | | | 1% | 3% | 5% | 1% | 3% | 5% |
|---|---|---|---|---|---|---|---|---|---|
| number | Residues | Sequence | Iscore | hits | hits | hits | pop | pop | pop |
| Ag. A1 | 163-171 | YTFVPWLLS | 2.8 | 0 | 0 | 1 | 0.00 | 0.00 | 0.11 |
| Ag. A2 | 168-176 | WLLSFKRGS | 24.2 | 2 | 4 | 4 | 0.10 | 0.29 | 0.29 |
| Ag. A3 | 169-177 | LLSFKRGSA | 14.3 | 0 | 1 | 2 | 0.00 | 0.23 | 0.24 |
| Ag. A4 | 185-193 | ILVKETGYF | 1.3 | 0 | 0 | 1 | 0.00 | 0.00 | 0.05 |
| Ag. A5 | 186-194 | LVKETGYFF | 1.2 | 0 | 0 | 1 | 0.00 | 0.00 | 0.05 |
| Ag. A6 | 192-200 | YFFIYGQVL | 42.5 | 2 | 2 | 3 | 0.34 | 0.34 | 0.37 |
| Ag. A7 | 193-201 | FFIYGQVLY | 3.6 | 0 | 1 | 2 | 0.00 | 0.05 | 0.07 |
| Ag. A8 | 194-202 | FIYGQVLYT | 0.5 | 0 | 0 | 1 | 0.00 | 0.00 | 0.02 |
| Ag. A9 | 200-208 | LYTDKTYAM | 26.2 | 1 | 1 | 1 | 0.21 | 0.21 | 0.21 |
| Ag. A10 | 212-220 | IQRKKVHVF | 26.4 | 2 | 4 | 5 | 0.19 | 0.24 | 0.25 |
| Ag. A11 | 219-227 | VFGDELSLV | 5.2 | 0 | 0 | 1 | 0.00 | 0.00 | 0.21 |
| Ag. A12 | 226-234 | LVTLFRCIQ | 27.9 | 4 | 4 | 5 | 0.21 | 0.21 | 0.29 |
| Ag. A13 | 227-235 | VTLFRCIQN | 5.6 | 0 | 0 | 1 | 0.00 | 0.00 | 0.23 |
| Ag. A14 | 230-238 | FRCIQNMPE | 16.6 | 2 | 3 | 7 | 0.03 | 0.17 | 0.35 |
| Ag. A15 | 259-267 | LQLAIPREN | 3.3 | 0 | 0 | 3 | 0.00 | 0.00 | 0.14 |
| Ag. A16 | 276-284 | VTFFGALKL | 40.9 | 1 | 3 | 4 | 0.23 | 0.43 | 0.46 |

TABLE 2.B
Predicted MHC-binding agretopes in RANKL.

| Agretope | | | | 1% | 3% | 5% | 1% | 3% | 5% |
|---|---|---|---|---|---|---|---|---|---|
| number | Residues | Sequence | Iscore | hits | hits | hits | pop | pop | pop |
| Ag. B1 | 193-201 | WAKISNMTF | 0.7 | 0 | 0 | 2 | 0% | 0% | 3% |
| Ag. B2 | 207-215 | IVNQDGFYY | 8.1 | 0 | 1 | 3 | 0% | 5% | 25% |
| Ag. B3 | 213-221 | FYYLYANIC | 27.9 | 2 | 5 | 6 | 13% | 31% | 35% |
| Ag. B4 | 214-222 | YYLYANICF | 34.7 | 2 | 2 | 5 | 24% | 24% | 45% |
| Ag. B5 | 215-223 | YLYANICFR | 5.5 | 0 | 1 | 1 | 0% | 9% | 9% |
| Ag. B6 | 222-230 | FRHHETSGD | 15.5 | 1 | 3 | 4 | 2% | 23% | 24% |
| Ag. B7 | 235-243 | YLQLMVYVT | 3.0 | 0 | 0 | 1 | 0% | 0% | 12% |
| Ag. B8 | 236-244 | LQLMVYVTK | 1.5 | 0 | 0 | 1 | 0% | 0% | 6% |
| Ag. B9 | 238-246 | LMVYVTKTS | 13.9 | 0 | 4 | 6 | 0% | 18% | 30% |
| Ag. B10 | 239-247 | MVYVTKTSI | 44.6 | 1 | 3 | 6 | 21% | 43% | 63% |
| Ag. B11 | 240-248 | VYVTKTSIK | 4.7 | 0 | 0 | 2 | 0% | 0% | 19% |
| Ag. B12 | 241-249 | YVTKTSIKI | 35.4 | 1 | 3 | 5 | 25% | 30% | 37% |
| Ag. B13 | 247-255 | IKIPSSHTL | 10.3 | 0 | 0 | 2 | 0% | 0% | 42% |
| Ag. B14 | 270-278 | FHFYSINVG | 9.4 | 0 | 3 | 3 | 0% | 15% | 15% |
| Ag. B15 | 277-285 | VGGFFKLRS | 45.1 | 4 | 6 | 7 | 26% | 48% | 49% |
| Ag. B16 | 280-288 | FFKLRSGEE | 0.4 | 0 | 0 | 1 | 0% | 0% | 2% |
| Ag. B17 | 289-297 | ISIEVSNPS | 18.5 | 1 | 1 | 2 | 14% | 14% | 19% |
| Ag. B18 | 308-316 | FGAFKVRDI | 18.5 | 1 | 1 | 4 | 9% | 9% | 40% |

TABLE 2.C
Predicted MHC-binding agretopes in APRIL.

| Agretope | | | | 1% | 3% | 5% | 1% | 3% | 5% |
|---|---|---|---|---|---|---|---|---|---|
| number | Residues | Sequence | Iscore | hits | hits | hits | pop | pop | pop |
| Ag. C1 | 117-125 | VLHLVPINA | 22.2 | 1 | 4 | 5 | 5% | 30% | 34% |
| Ag. C2 | 120-128 | LVPINATSK | 4.3 | 0 | 2 | 2 | 0% | 7% | 7% |
| Ag. C3 | 121-129 | VPINATSKD | 0.4 | 0 | 0 | 1 | 0% | 0% | 2% |
| Ag. C4 | 138-146 | WQPALRRGR | 13.3 | 1 | 1 | 2 | 9% | 9% | 19% |
| Ag. C5 | 142-150 | LRRGRGLQA | 36.5 | 1 | 4 | 4 | 23% | 37% | 37% |
| Ag. C6 | 155-163 | VRIQDAGVY | 17.8 | 0 | 2 | 3 | 0% | 25% | 34% |
| Ag. C7 | 162-170 | VYLLYSQVL | 19.5 | 1 | 2 | 5 | 5% | 17% | 42% |
| Ag. C8 | 163-171 | YLLYSQVLF | 3.8 | 0 | 0 | 3 | 0% | 0% | 15% |
| Ag. C9 | 164-172 | LLYSQVLFQ | 35.4 | 2 | 6 | 8 | 18% | 34% | 49% |
| Ag. C10 | 170-178 | LFQDVTFTM | 26.2 | 1 | 1 | 1 | 21% | 21% | 21% |
| Ag. C11 | 194-202 | FRCIRSMPS | 56.7 | 7 | 9 | 14 | 35% | 44% | 78% |
| Ag. C12 | 197-205 | IRSMPSHPD | 7.6 | 1 | 2 | 5 | 2% | 8% | 15% |
| Ag. C13 | 217-225 | FHLHQGDIL | 0.5 | 0 | 0 | 1 | 0% | 0% | 2% |
| Ag. C14 | 227-235 | VIIPRARAK | 0.4 | 0 | 0 | 1 | 0% | 0% | 2% |
| Ag. C15 | 228-236 | IIPRARAKL | 3.1 | 0 | 1 | 1 | 0% | 5% | 5% |
| Ag. C16 | 236-244 | LNLSPHGTF | 5.2 | 0 | 0 | 1 | 0% | 0% | 21% |
| Ag. C17 | 238-246 | LSPHGTFLG | 3.0 | 0 | 0 | 2 | 0% | 0% | 12% |

TABLE 2.D
Predicted MHC-binding agretopes in CD40L.

| Agretope | | | | 1% | 3% | 5% | 1% | 3% | 5% |
|---|---|---|---|---|---|---|---|---|---|
| number | Residues | Sequence | Iscore | hits | hits | hits | pop | pop | pop |
| Ag. D1 | 145-153 | YYTMSNNLV | 17.74 | 2 | 4 | 6 | 0.03 | 0.22 | 0.33 |
| Ag. D2 | 146-154 | YTMSNNLVT | 14.14 | 0 | 1 | 3 | 0.00 | 0.14 | 0.37 |
| Ag. D3 | 152-160 | LVTLENGKQ | 9.35 | 0 | 3 | 5 | 0.00 | 0.09 | 0.25 |
| Ag. D4 | 168-176 | LYYIYAQVT | 10.25 | 0 | 2 | 2 | 0.00 | 0.17 | 0.17 |
| Ag. D5 | 169-177 | YYIYAQVTF | 7.7 | 0 | 2 | 3 | 0.00 | 0.11 | 0.15 |
| Ag. D6 | 170-178 | YIYAQVTFC | 13.76 | 0 | 3 | 5 | 0.00 | 0.17 | 0.31 |
| Ag. D7 | 171-179 | IYAQVTFCS | 18.95 | 0 | 6 | 7 | 0.00 | 0.27 | 0.37 |
| Ag. D8 | 175-183 | VTFCSNREA | 1.04 | 0 | 1 | 1 | 0.00 | 0.02 | 0.02 |
| Ag. D9 | 189-197 | FIASLCLKS | 14.62 | 0 | 2 | 2 | 0.00 | 0.24 | 0.24 |
| Ag. D10 | 204-212 | ILLRAANTH | 4.51 | 0 | 2 | 3 | 0.00 | 0.07 | 0.08 |
| Ag. D11 | 205-213 | LLRAANTHS | 12.4 | 1 | 3 | 5 | 0.06 | 0.09 | 0.23 |
| Ag. D12 | 206-214 | LRAANTHSS | 48.23 | 6 | 10 | 10 | 0.31 | 0.48 | 0.48 |
| Ag. D13 | 223-231 | IHLGGVFEL | 17.86 | 0 | 1 | 2 | 0.00 | 0.21 | 0.41 |
| Ag. D14 | 229-237 | FELQPGASV | 7.56 | 0 | 1 | 1 | 0.00 | 0.12 | 0.12 |
| Ag. D15 | 237-245 | VFVNVTDPS | 8.6 | 0 | 1 | 1 | 0.00 | 0.14 | 0.14 |
| Ag. D16 | 253-261 | FTSFGLLKL | 12.5 | 1 | 1 | 3 | 0.02 | 0.02 | 0.43 |

TABLE 2.E
Predicted MHC-binding agretopes in TRAIL.

| Agretope | | | | 1% | 3% | 5% | 1% | 3% | 5% |
|---|---|---|---|---|---|---|---|---|---|
| number | Residues | Sequence | Iscore | hits | hits | hits | pop | pop | pop |
| Ag. E1 | 151-159 | INSWESSRS | 5.9 | 1 | 2 | 3 | 2% | 8% | 9% |
| Ag. E2 | 174-182 | LVIHEKGFY | 46.1 | 4 | 4 | 5 | 37% | 37% | 40% |
| Ag. E3 | 181-189 | FYYIYSQTY | 21.0 | 0 | 3 | 5 | 0% | 27% | 45% |
| Ag. E4 | 182-190 | YYIYSQTYF | 1.2 | 0 | 1 | 1 | 0% | 2% | 2% |
| Ag. E5 | 183-191 | YIYSQTYFR | 3.7 | 0 | 0 | 2 | 0% | 0% | 15% |
| Ag. E6 | 206-214 | MVQYIYKYT | 14.3 | 0 | 1 | 2 | 0% | 23% | 24% |
| Ag. E7 | 207-215 | VQYIYKYTS | 47.2 | 3 | 5 | 6 | 33% | 41% | 47% |
| Ag. E8 | 209-217 | YIYKYTSYP | 1.3 | 0 | 0 | 1 | 0% | 0% | 5% |
| Ag. E9 | 220-228 | ILLMKSARN | 7.6 | 1 | 3 | 4 | 2% | 9% | 13% |
| Ag. E10 | 221-229 | LLMKSARNS | 28.5 | 4 | 5 | 5 | 21% | 25% | 25% |
| Ag. E11 | 223-231 | MKSARNSCW | 1.5 | 0 | 0 | 1 | 0% | 0% | 6% |
| Ag. E12 | 237-245 | YGLYSIYQG | 6.3 | 0 | 2 | 3 | 0% | 7% | 15% |
| Ag. E13 | 240-248 | YSIYQGGIF | 1.2 | 0 | 1 | 1 | 0% | 2% | 2% |
| Ag. E14 | 256-264 | IFVSVTNEH | 21.8 | 1 | 3 | 5 | 14% | 21% | 23% |
| Ag. E15 | 257-265 | FVSVTNEHL | 33.9 | 1 | 3 | 4 | 25% | 27% | 36% |

Table 3. Predicted MHC-binding agretopes in TNF SF members. DRB1 alleles that are predicted to bind to each allele at 1%, 3%, 5% and 10% cutoffs are markd with “1”, “3”, “5” or “10” respectively.

TABLE 3.A
Alleles predicted to bind MHC agretopes in BAFF.

Agretope

| number | 101 | 102 | 301 | 401 | 402 | 404 | 405 | 408 | 701 | 801 | 1101 | 1102 | 1104 | 1301 | 1302 | 1501 | 1502 |
|---|---|---|---|---|---|---|---|---|---|---|---|---|---|---|---|---|---|
| Ag. A1 | — | — | — | — | — | — | — | 10 | — | — | 5 | — | — | — | — | — | — |
| Ag. A2 | — | — | 10 | — | — | — | — | — | — | 10 | 3 | 1 | 10 | 3 | 1 | — | — |
| Ag. A3 | — | — | — | — | — | — | — | — | — | — | 10 | — | 10 | — | — | 3 | 5 |
| Ag. A4 | — | — | — | — | — | — | — | — | — | 5 | — | — | — | 10 | — | — | — |
| Ag. A5 | 10 | 5 | — | — | — | — | — | — | — | — | — | — | — | — | — | — | — |
| Ag. A6 | 1 | 5 | — | — | — | — | — | — | 1 | — | — | — | — | — | — | — | 10 |
| Ag. A7 | — | — | — | — | — | — | — | — | — | 3 | — | — | — | — | 10 | — | 5 |
| Ag. A8 | 10 | — | — | — | — | — | — | — | — | — | 10 | — | — | — | — | — | 5 |
| Ag. A9 | — | — | 1 | 10 | — | — | — | — | — | — | — | — | — | — | — | — | — |
| Ag. A10 | — | — | — | — | 5 | — | — | — | — | 3 | — | 3 | — | 1 | 1 | — | — |
| Ag. A11 | — | — | 5 | — | — | — | — | — | — | — | — | — | — | — | — | — | — |
| Ag. A12 | — | — | 10 | — | — | — | — | — | — | 1 | 1 | 1 | 1 | 5 | 10 | — | — |
| Ag. A13 | — | — | — | — | — | — | — | — | — | — | — | — | — | — | — | 5 | 10 |
| Ag. A14 | — | — | — | 3 | — | 5 | 1 | 1 | 10 | 5 | 5 | — | — | — | — | — | 5 |
| Ag. A15 | — | 10 | — | — | 5 | — | — | — | — | — | — | 5 | — | 5 | 10 | — | — |
| Ag. A16 | 10 | 5 | — | — | — | — | — | — | 3 | — | — | — | 10 | — | — | 1 | 3 |

TABLE 3.B
Alleles predicted to bind MHC agretopes in RANKL.

Agretope

| number | 101 | 102 | 301 | 401 | 402 | 404 | 405 | 408 | 701 | 801 | 1101 | 1102 | 1104 | 1301 | 1302 | 1501 | 1502 |
|---|---|---|---|---|---|---|---|---|---|---|---|---|---|---|---|---|---|
| Ag. B1 | — | — | — | 10 | — | — | 5 | 5 | 10 | — | — | — | — | — | — | — | 10 |
| Ag. B2 | 5 | 3 | — | — | — | — | — | — | — | — | — | 10 | — | 5 | 10 | — | — |
| Ag. B3 | 1 | 3 | — | 3 | — | 5 | 3 | 1 | — | — | 10 | — | — | — | 10 | — | 10 |
| Ag. B4 | — | — | — | — | — | — | 5 | 5 | 5 | 10 | — | — | — | — | 10 | 1 | 1 |
| Ag. B5 | — | — | — | — | — | — | — | — | — | — | 10 | — | — | — | 3 | — | — |
| Ag. B6 | — | — | — | 3 | 10 | — | 1 | 5 | — | 10 | — | — | — | — | 3 | — | — |
| Ag. B7 | 5 | — | — | — | — | — | 10 | 10 | — | — | 10 | — | — | — | — | — | — |
| Ag. B8 | — | — | — | — | — | 5 | — | 10 | — | — | — | — | — | — | — | — | — |
| Ag. B9 | — | — | — | — | 3 | 5 | — | 10 | — | 3 | 10 | 3 | 10 | 3 | 5 | 10 | — |
| Ag. B10 | 10 | 5 | 1 | — | — | — | — | — | 3 | — | 5 | 10 | 3 | 10 | 10 | 5 | 10 |
| Ag. B11 | — | — | — | 5 | 10 | 5 | — | 10 | — | — | — | — | — | — | — | — | — |
| Ag. B12 | 10 | — | — | — | 5 | — | — | 10 | 1 | 3 | 10 | — | — | — | 5 | 10 | 3 |
| Ag. B13 | — | 10 | — | — | 10 | — | — | — | 5 | — | — | — | — | — | — | 5 | 10 |
| Ag. B14 | — | — | — | — | — | — | 10 | — | — | 3 | — | — | — | — | 3 | 10 | 3 |
| Ag. B15 | — | — | — | — | — | — | — | — | — | 10 | 1 | 1 | 1 | 1 | 3 | 3 | 5 |
| Ag. B16 | — | — | — | — | — | — | 5 | — | — | 10 | — | — | — | — | — | — | — |
| Ag. B17 | — | — | — | 1 | — | 5 | — | 10 | — | — | — | — | — | — | — | — | — |
| Ag. B18 | 10 | — | — | — | 10 | — | — | 10 | 5 | — | 10 | 5 | — | 5 | 1 | — | 10 |

TABLE 3.C
Alleles predicted to bind MHC agretopes in APRIL.

Agretope

| number | 101 | 102 | 301 | 401 | 402 | 404 | 405 | 408 | 701 | 801 | 1101 | 1102 | 1104 | 1301 | 1302 | 1501 | 1502 |
|---|---|---|---|---|---|---|---|---|---|---|---|---|---|---|---|---|---|
| Ag. C1 | 3 | 1 | — | — | — | 5 | — | 10 | — | — | 3 | 10 | 3 | — | — | 10 | — |
| Ag. C2 | 10 | 10 | — | 10 | 10 | 3 | 10 | 3 | — | — | — | — | — | — | — | — | — |
| Ag. C3 | — | — | — | — | — | — | 5 | — | — | — | — | — | — | — | — | — | — |
| Ag. C4 | — | — | — | — | — | — | — | — | — | — | — | 10 | — | 5 | 1 | — | — |
| Ag. C5 | 3 | 3 | 10 | — | 10 | — | — | — | — | — | — | — | 10 | — | — | 1 | 3 |
| Ag. C6 | 5 | 3 | 3 | — | — | 10 | — | — | — | — | — | 10 | 10 | 10 | 10 | — | — |
| Ag. C7 | 3 | 1 | — | 10 | 5 | 5 | 10 | 10 | 5 | — | — | — | — | — | — | 10 | — |
| Ag. C8 | — | — | — | — | — | — | — | — | — | 5 | — | — | — | — | 5 | — | 5 |
| Ag. C9 | — | — | 5 | 1 | 3 | 3 | 10 | 5 | — | 10 | 3 | 3 | 1 | 10 | — | — | — |
| Ag. C10 | — | — | 1 | 10 | — | — | — | — | — | — | — | — | — | — | — | — | — |
| Ag. C11 | 3 | 10 | 5 | 1 | 3 | 1 | 1 | 1 | 5 | 5 | 1 | — | 1 | — | 5 | 5 | 1 |
| Ag. C12 | — | 10 | — | 10 | 5 | 3 | 1 | 5 | — | 5 | — | 10 | — | 10 | — | — | — |
| Ag. C13 | — | — | — | — | — | — | — | — | — | — | — | — | — | — | — | — | 5 |
| Ag. C14 | — | — | — | — | 5 | — | — | — | — | — | — | 10 | — | — | — | — | — |
| Ag. C15 | — | — | — | — | — | — | — | — | — | 3 | — | 10 | — | 10 | — | 10 | — |
| Ag. C16 | — | — | 5 | — | — | — | — | — | — | — | — | — | — | — | — | — | — |
| Ag. C17 | — | — | — | — | 5 | — | — | — | — | — | — | 10 | — | 5 | 10 | — | — |

TABLE 3.D
Alleles predicted to bind MHC agretopes in CD40L.

Agretope

| number | 101 | 102 | 301 | 401 | 402 | 404 | 405 | 408 | 701 | 801 | 1101 | 1102 | 1104 | 1301 | 1302 | 1501 | 1502 |
|---|---|---|---|---|---|---|---|---|---|---|---|---|---|---|---|---|---|
| Ag. D1 | 5 | — | — | 3 | — | 3 | 1 | 1 | — | — | — | — | — | — | — | — | 5 |
| Ag. D2 | — | — | — | 3 | — | — | — | — | 5 | — | — | — | — | — | — | — | 5 |
| Ag. D3 | — | — | — | 5 | — | 3 | 3 | 3 | — | — | — | — | 5 | — | — | — | — |
| Ag. D4 | 3 | 3 | — | — | — | — | — | — | — | — | — | — | — | — | — | — | — |
| Ag. D5 | — | — | — | — | — | — | — | — | — | 5 | — | — | — | — | 3 | — | 3 |
| Ag. D6 | 5 | — | — | 3 | — | 5 | 3 | 3 | — | — | — | — | — | — | — | — | — |
| Ag. D7 | — | — | — | 5 | 3 | 3 | — | 3 | — | — | — | 3 | — | 3 | 3 | — | — |
| Ag. D8 | — | — | — | — | 3 | — | — | — | — | — | — | — | — | — | — | — | — |
| Ag. D9 | — | — | — | 3 | — | — | — | — | — | — | 3 | — | — | — | — | — | — |
| Ag. D10 | — | — | — | — | 3 | — | — | — | — | 3 | — | 5 | — | — | — | — | — |
| Ag. D11 | — | — | — | 5 | 3 | 1 | 5 | 3 | — | — | — | — | — | — | — | — | — |
| Ag. D12 | — | — | — | 1 | 1 | 1 | 3 | 1 | — | — | 3 | 1 | 3 | 1 | 3 | — | — |
| Ag. D13 | — | — | 3 | — | — | — | — | — | 5 | — | — | — | — | — | — | — | — |
| Ag. D14 | 3 | — | — | — | — | — | — | — | — | — | — | — | — | — | — | — | — |
| Ag. D15 | — | — | — | 3 | — | — | — | — | — | — | — | — | — | — | — | — | — |
| Ag. D16 | — | — | — | — | — | — | — | — | 5 | — | — | — | — | — | — | 5 | 1 |

TABLE 3.E
Alleles predicted to bind MHC agretopes in TRAIL.

Agretope

| number | 101 | 102 | 301 | 401 | 402 | 404 | 405 | 408 | 701 | 801 | 1101 | 1102 | 1104 | 1301 | 1302 | 1501 | 1502 |
|---|---|---|---|---|---|---|---|---|---|---|---|---|---|---|---|---|---|
| Ag. E1 | — | — | — | — | 1 | 3 | 10 | 5 | — | — | — | — | — | — | — | — | — |
| Ag. E2 | — | — | 1 | — | — | — | — | — | — | 10 | 10 | 1 | 5 | 1 | 1 | 10 | 10 |
| Ag. E3 | 5 | — | — | 5 | 10 | 10 | 3 | 3 | 3 | — | — | — | — | — | — | — | — |
| Ag. E4 | — | — | — | — | — | — | — | — | — | — | — | — | — | — | 10 | 10 | 3 |
| Ag. E5 | — | — | 10 | 5 | 10 | — | 10 | 5 | 10 | — | — | — | — | — | 10 | — | — |
| Ag. E6 | — | — | — | — | — | — | — | — | — | — | — | — | — | — | — | 3 | 5 |
| Ag. E7 | — | 10 | 1 | — | — | — | — | — | — | 10 | 1 | 3 | 1 | 3 | 5 | — | — |
| Ag. E8 | — | — | — | — | — | — | 10 | 10 | — | 5 | — | — | — | — | 10 | — | — |
| Ag. E9 | 10 | 10 | — | 10 | 10 | 3 | 1 | 3 | — | 5 | — | — | — | — | — | — | — |
| Ag. E10 | — | — | — | — | 1 | — | — | — | — | 3 | — | 1 | 10 | 1 | 1 | — | — |
| Ag. E11 | — | — | — | — | — | 5 | 10 | 10 | — | — | — | — | — | — | — | — | — |
| Ag. E12 | — | — | — | — | — | — | — | — | — | 3 | — | — | — | — | 5 | 10 | 3 |
| Ag. E13 | — | — | — | — | — | — | — | — | — | 10 | — | — | — | — | — | — | 3 |
| Ag. E14 | — | — | — | 1 | 5 | 3 | 3 | 5 | — | — | — | 10 | — | — | — | — | — |
| Ag. E15 | 5 | — | — | 10 | — | — | 3 | 3 | 1 | — | — | — | — | — | — | — | 10 |

### Example 2

### Identification of Suitable Less Immunogenic Sequences for MHC-Binding AgretoDes in TNF SF Members

MHC-binding agretopes that were predicted to bind alleles present in at least 10% of the US population, using a 1% threshold, were analyzed to identify suitable less immunogenic variants. At each agretope, all possible combinations of amino acid substitutions were considered, with the following requirements: (1) each substitution has a score of 0 or greater in the BLOSUM62 substitution matrix, (2) each substitution is capable of conferring reduced binding to at least one of the MHC alleles considered, and (3) once sufficient substitutions are incorporated to prevent any allele hits at a 1% threshold, no additional substitutions are added to that sequence.

Alternate sequences were scored for immunogenicity and structural compatibility. Preferred alternate sequences were defined to be those sequences that are not predicted to bind to any of the 17 MHC alleles tested above using a 1% threshold, and that have a total BLOSUM62 score that is at least 80% of the wild type score.

Table 4. Suitable less immunogenic variants of of TNF SF members. B(wt) is the BLOSUM62 score of the wild type 9-mer, l(alt) is the percent of the US population containing one or more MHC alleles that are predicted to bind the alternate 9-mer at a 1% threshold and is 0 for all variants listed in Table 4, and B(alt) is the BLOSUM62 score of the alternate 9-mer.

TABLE 4A.i

Suitable less immunogenic variants of BAFF
agretope 2 (residues 168-176; WLLSFKRGS);
B (wt) = 49.

| | | |
|---|---|---|
| | Variant | |
| Variant | sequence | B (alt) |

| | | |
|---|---|---|
| Var:A1 | WLLSFSRGS | 44 |
| Var:A2 | WLLSFNRGS | 44 |
| Var:A3 | WLLSFERGS | 45 |
| Var:A4 | WLLSFQRGS | 45 |
| Var:A5 | WLLSFKEGS | 44 |
| Var:A6 | WLLSFKQGS | 45 |
| Var:A7 | WLLSFKRGT | 46 |
| Var:A8 | WLLSFKRGN | 46 |
| Var:A9 | WLLSFKRGD | 45 |
| Var:A10 | WLLSFKRGE | 45 |
| Var:A11 | WLLSFKRGQ | 45 |
| Var:A12 | WLLSFKRGK | 45 |
| Var:A13 | WFLSFKNGS | 40 |
| Var:A14 | WFLSFKKGS | 42 |
| Var:A15 | WFLSFKRGA | 42 |
| Var:A16 | WLVGFKRGS | 42 |
| Var:A17 | WLVDFKRGS | 42 |
| Var:A18 | WLVSFKNGS | 41 |
| Var:A19 | WLVSFKHGS | 41 |
| Var:A20 | WLVSFKKGS | 43 |
| Var:A21 | WLVSFKRGA | 43 |
| Var:A22 | WLFSFKNGS | 40 |
| Var:A23 | WLFSFKKGS | 42 |
| Var:A24 | WLFSFKRGA | 42 |
| Var A25 | WLLTFKNGS | 41 |
| Var:A26 | WLLTFKHGS | 41 |
| Var:A27 | WLLTFKKGS | 43 |
| Var:A28 | WLLTFKRGA | 43 |
| Var:A29 | WLLGFKNGS | 40 |
| Var:A30 | WLLGFKHGS | 40 |
| Var:A31 | WLLGFKKGS | 42 |
| Var:A32 | WLLGFKRGA | 42 |
| Var:A33 | WLLGFKRGG | 41 |
| Var:A34 | WLLDFKRGA | 42 |
| Var:A35 | WLLDFKRGG | 41 |
| Var:A36 | WLLEFKNGS | 40 |
| Var:A37 | WLLEFKHGS | 40 |
| Var:A38 | WLLEFKKGS | 42 |
| Var:A39 | WLLEFKRGA | 42 |
| Var:A40 | WLLSFRNGS | 41 |
| Var:A41 | WLLSFRKGS | 43 |
| Var:A42 | WLLSFRRGA | 43 |
| Var:A43 | WLLSFKNGA | 41 |
| Var:A44 | WLLSFKNGG | 40 |
| Var:A45 | WLLSFKHGA | 41 |
| Var:A46 | WLLSFKHGG | 40 |
| Var:A47 | WLLSFKKGA | 43 |
| Var:A48 | WLLSFKKGG | 42 |
| Var:A49 | WLVTFRRGS | 40 |

TABLE 4.A.ii

lists suitable less immunogenic variants of BAFF
agretope 6 (residues 192-200; YFFIYGQVL);
B (wt) = 49.

| | | |
|---|---|---|
| | Variant | |
| Variant | sequence | B (alt) |

| | | |
|---|---|---|
| Var:A52 | YFFVYGQVL | 48 |
| Var:A53 | YFFIYGDVL | 44 |
| Var:A54 | YFFIYGEVL | 46 |
| Var:A55 | YFFIYGQVM | 47 |
| Var:A56 | YFFIYGQVF | 45 |
| Var:A57 | YFFLYNQVL | 41 |
| Var:A50 | YWFIYGQVL | 44 |
| Var:A51 | YFWIYGQVL | 44 |
| Var:A58 | YFFFYGRVL | 41 |
| Var:A59 | YFFFYGQVV | 42 |
| Var:A60 | YFFIYSQVV | 40 |
| Var:A61 | YFFIYNQVV | 40 |
| Var:A62 | YFFIYGSVV | 41 |
| Var:A63 | YFFIYGKVV | 42 |

TABLE 4.A.iii

Suitable less immunogenic variants of BAFF
agretope 9 (residues 200-208 LYTDKTYAM);
B (wt) = 48.

| | | |
|---|---|---|
| | Variant | |
| Variant | sequence | B (alt) |

| | | |
|---|---|---|
| Var:A64 | FYTDKTYAM | 44 |
| Var:A65 | LWTDKTYAM | 43 |
| Var:A66 | LYTSKTYAM | 42 |
| Var:A67 | LYTNKTYAM | 43 |
| Var:A68 | LYTEKTYAM | 44 |
| Var:A69 | LYTQKTYAM | 42 |
| Var:A70 | LYTDKSYAM | 44 |
| Var:A71 | LYTDKAYAM | 43 |
| Var:A72 | LYTDKNYAM | 43 |
| Var:A73 | LYTDKTHAM | 43 |
| Var:A74 | LYTDKTWAM | 43 |
| Var:A75 | LYTDKTYAQ | 44 |
| Var:A76 | LYTDKTYAI | 44 |
| Var:A77 | LYTDKTYAL | 45 |
| Var:A78 | LYTDKTYAV | 44 |

TABLE 4.A.iv

Suitable less immunogenic variants of BAFF
agretope 10 (residues 212-220; IQRKKVHVF);
B (wt) = 46.

| | | |
|---|---|---|
| | Variant | |
| Variant | sequence | B (alt) |

| | | |
|---|---|---|
| Var:A79 | ISRKKVHVF | 41 |
| Var:A80 | IDRKKVHVF | 41 |
| Var:A81 | IERKKVHVF | 43 |
| Var:A82 | IQEKKVHVF | 41 |
| Var:A83 | IQRSKVHVF | 41 |
| Var:A84 | IQREKVHVF | 42 |
| Var:A85 | IQRKKAHVF | 43 |
| Var:A86 | IQRKKMHVF | 43 |
| Var:A87 | IQRKKLHVF | 43 |
| Var:A88 | IQRKKVEVF | 38 |
| Var:A89 | IQRKKVHVL | 40 |
| Var:A90 | IQRKKVHVW | 41 |
| Var:A91 | INQKKVHVF | 37 |
| Var:A92 | INKKKVHVF | 38 |
| Var:A93 | INRRKVHVF | 38 |
| Var:A94 | INRKKIHVF | 40 |
| Var:A95 | INRKKVHVY | 38 |
| Var:A96 | IHQKKVHVF | 37 |
| Var:A97 | IHKKKVHVF | 38 |
| Var:A98 | IHRRKVHVF | 38 |
| Var:A99 | IHRKKIHVF | 40 |
| Var:A100 | IHRKKVHVY | 38 |
| Var:A101 | IQQNKVHVF | 37 |
| Var:A102 | IQQQKVHVF | 38 |
| Var:A103 | IQQRKVHVF | 39 |
| Var:A104 | IQQKKTHVF | 39 |
| Var:A105 | IQQKKIHVF | 41 |
| Var:A106 | IQQKKVHVY | 39 |
| Var:A107 | IQHRKVHVF | 38 |
| Var:A108 | IQHKKTHVF | 38 |
| Var:A109 | IQHKKIHVF | 40 |
| Var:A110 | IQHKKVHVY | 38 |
| Var:A111 | IQKNKVHVF | 38 |
| Var:A112 | IQKQKVHVF | 39 |
| Var:A113 | IQKRKVHVF | 40 |
| Var:A114 | IQKKKTHVF | 40 |
| Var:A115 | IQKKKIHVF | 42 |
| Var:A116 | IQKKKVYVF | 37 |
| Var:A117 | IQKKKVHVI | 37 |
| Var:A118 | IQKKKVHVY | 40 |
| Var:A119 | IQRNKTHVF | 38 |
| Var:A120 | IQRNKIHVF | 40 |
| Var:A121 | IQRNKVHVY | 38 |
| Var:A122 | IQRRKIHVF | 42 |
| Var:A123 | IQRRKVYVF | 37 |
| Var:A124 | IQRRKVHVY | 40 |
| Var:A125 | IQRKKTYVF | 37 |
| Var:A126 | IQRKKTHVY | 40 |
| Var:A127 | IQRKKIQVF | 37 |
| Var:A128 | IQRKKIYVF | 39 |
| Var:A129 | IQRKKIHVY | 42 |
| Var:A130 | IQRKKVYVY | 37 |

TABLE 4.A.v

Suitable less immunogenic variants of BAFF
agretope 12 (residues 226-234; LVTLFRCIQ);
B (wt) = 46.

| | | |
|---|---|---|
| | Variant | |
| Variant | sequence | B (alt) |

| | | |
|---|---|---|
| Var:A131 | LTTLFRCIQ | 43 |
| Var:A132 | LATLFRCIQ | 43 |
| Var:A133 | LMTLFRCIQ | 43 |
| Var:A134 | LITLFRCIQ | 45 |
| Var:A135 | LLTLFRCIQ | 43 |
| Var:A136 | LVTLFECIQ | 41 |
| Var:A137 | LVTLFQCIQ | 42 |
| Var:A138 | LVTLFHCIQ | 41 |
| Var:A139 | LVTLFRCIN | 41 |
| Var:A140 | LVTLFRCID | 41 |
| Var:A141 | LVTLFRCIR | 42 |
| Var:A142 | LVTIFRCIE | 41 |
| Var:A143 | LVTIFRCIK | 40 |
| Var:A144 | LVTVFRCIE | 40 |
| Var:A145 | LVTVFRCIH | 38 |
| Var:A146 | LVTVFRCIK | 39 |
| Var:A147 | LVTVFRCIM | 38 |
| Var:A148 | LVTFFRCIE | 39 |

TABLE 4.A.vi

Suitable less immunogenic variants of BAFF
agretope 16 (residues 276-284; VTFFGALKL);
B (wt) = 44.

| | | |
|---|---|---|
| | Variant | |
| Variant | sequence | B (alt) |

| | | |
|---|---|---|
| Var:A149 | VTWFGALKL | 39 |
| Var:A150 | VTFMGALKL | 38 |
| Var:A151 | VTFIGALKL | 38 |
| Var:A152 | VTFWGALKL | 39 |
| Var:A153 | VTFFGAVKL | 41 |
| Var:A154 | VTFFGALKF | 40 |
| Var:A155 | VSFFGVLKL | 36 |
| Var:A156 | VSFFGAIKL | 38 |
| Var:A157 | VSFFGAFKL | 36 |
| Var:A158 | VSFFGALKM | 38 |
| Var:A159 | VTFLGAMKL | 36 |
| Var:A160 | VTFLGALKM | 36 |
| Var:A161 | VTFFGTFKL | 36 |
| Var:A162 | VTFFGVIKL | 38 |
| Var:A163 | VTFFGVFKL | 36 |
| Var:A164 | VTFFGAMKM | 40 |
| Var:A165 | VTFFGAIKM | 40 |
| Var:A166 | VTFFGAIKV | 39 |
| Var:A167 | VTFFGAFKM | 38 |
| Var:A168 | VTFFGAFKV | 37 |

TABLE 4.B.i

Suitable less immunogenic variants of RANKL
agretope 3 (residues 213-221; FYYLYANIC);
B (wt) = 54.

| | | |
|---|---|---|
| | Variant | |
| Variant | sequence | B (alt) |

| | | |
|---|---|---|
| Var:B1 | EWYLYANIC | 49 |
| Var:B2 | FYWLYANIC | 49 |
| Var:B3 | FYYVYANIC | 51 |
| Var:B4 | FYYLYAGIC | 48 |
| Var:B5 | FYYLYADIC | 49 |
| Var:B6 | FYYLYAEIC | 48 |
| Var:B7 | MYYIYANIC | 46 |
| Var:B8 | MYYFYANIC | 44 |
| Var:B9 | MYYLYGNIC | 44 |
| Var:B10 | IYYIYANIC | 46 |
| Var:B11 | IYYFYANIC | 44 |
| Var:B12 | IYYLYGNIC | 44 |
| Var:B13 | LYYIYANIC | 46 |
| Var:B14 | LYYFYANIC | 44 |
| Var:B15 | LYYLYGNIC | 44 |
| Var:B16 | FHHLYANIC | 44 |
| Var:B17 | FFHLYANIC | 45 |
| Var:B18 | FYHIYANIC | 47 |
| Var:B19 | FYHFYANIC | 45 |
| Var:B20 | FYHLYGNIC | 45 |
| Var:B21 | FYHLYASIC | 44 |
| Var:B22 | FYYIYGNIC | 48 |
| Var:B23 | FYYIYASIC | 47 |
| Var:B24 | FYYIYAKIC | 46 |
| Var:B25 | FYYFYASIC | 45 |
| Var:B26 | FYYFYATIC | 44 |
| Var:B27 | FYYFYAQIC | 44 |
| Var:B28 | FYYFYAHIC | 45 |
| Var:B29 | FYYFYARIC | 44 |
| Var:B30 | FYYFYAKIC | 44 |
| Var:B31 | FYYLYSRIC | 45 |
| Var:B32 | FYYLYSKIC | 45 |

TABLE 4.B.ii

Suitable less immunogenic variants of RANKL
agretope 4 (residues 214-222; YYLYANICF);
B (wt) = 54.

| | | |
|---|---|---|
| | Variant | |
| Variant | sequence | B (alt) |

| | | |
|---|---|---|
| Var:B33 | YYLWANICF | 49 |
| Var:B34 | YYLYAEICF | 48 |
| Var:B35 | YFLYAQICF | 44 |
| Var:B36 | YWVYANICF | 46 |
| Var:B37 | YWLHANICF | 44 |
| Var:B38 | YWLYADICF | 44 |
| Var:B39 | YWLYAHICF | 44 |
| Var:B40 | YWLYANVCF | 48 |
| Var:B41 | YWLYANICY | 46 |
| Var:B42 | YWLYANICW | 44 |
| Var:B43 | YYVHANICF | 46 |
| Var:B44 | YYVYADICF | 46 |
| Var:B45 | YYVYAQICF | 45 |
| Var:B46 | YYVYAHICF | 46 |
| Var:B47 | YYVYANVCF | 50 |
| Var:B48 | YYVYANICW | 46 |
| Var:B49 | YYFHANICF | 45 |
| Var:B50 | YYFYAQICF | 44 |
| Var:B51 | YYLHADICF | 44 |
| Var:B52 | YYLHAHICF | 44 |
| Var:B53 | YYLHANVCF | 48 |
| Var:B54 | YYLHANICY | 46 |
| Var:B55 | YYLHANICW | 44 |
| Var:B56 | YYLFAQICF | 44 |
| Var:B57 | YYLYADVCF | 48 |
| Var:B58 | YYLYADICY | 46 |
| Var:B59 | YYLYADICW | 44 |
| Var:B60 | YYLYAQVCF | 47 |
| Var:B61 | YYLYAQFCF | 44 |
| Var:B62 | YYLYAQICY | 45 |
| Var:B63 | YYLYAHVCF | 48 |
| Var:B64 | YYLYAHICY | 46 |
| Var:B65 | YYLYAHICW | 44 |
| Var:B66 | YYLYANVCY | 50 |
| Var:B67 | YYLYANVCW | 48 |
| Var:B68 | YHFYANVCF | 44 |
| Var:B69 | YFFYANVCF | 45 |
| Var:B70 | YYFFANVCF | 45 |
| Var:B71 | YYFYASVCF | 44 |

TABLE 4.B.iii

Suitable less immunogenic variants of RANKL
agretope 10 (residues 239-247; MVYVTKTSI);
B (wt) = 43.

| | | |
|---|---|---|
| | Variant | |
| Variant | sequence | B (alt) |

| | | |
|---|---|---|
| Var:B72 | MTYVTKTSI | 40 |
| Var:B73 | MAYVTKTSI | 40 |
| Var:B74 | MMYVTKTSI | 40 |
| Var:B75 | MIYVTKTSI | 42 |
| Var:B76 | MLYVTKTSI | 40 |
| Var:B77 | MVHVTKTSI | 38 |
| Var:B78 | MVWVTKTSI | 38 |
| Var:B79 | MVYTTKTSI | 40 |
| Var:B80 | MVYVTETSI | 39 |
| Var:B81 | MVYVTQTSI | 39 |
| Var:B82 | MVYVTRTSI | 40 |
| Var:B83 | MVYVTKTSL | 41 |
| Var:B84 | MVYVTKTSV | 42 |
| Var:B85 | FVYVTKSSI | 35 |
| Var:B86 | FVYVTKTSM | 36 |
| Var:B87 | FVYVTKTSF | 35 |

TABLE 4.B.iv

Suitable less immunogenic variants of RANKL
agretope 12 (residues 212-220; YVTKTSIKI);
B (wt) = 43.

| | | |
|---|---|---|
| | Variant | |
| Variant | sequence | B (alt) |

| | | |
|---|---|---|
| Var:B88 | YTTKTSIKI | 40 |
| Var:B89 | YATKTSIKI | 40 |
| Var:B90 | YMTKTSIKI | 40 |
| Var:B91 | YITKTSIKI | 42 |
| Var:B92 | YLTKTSIKI | 40 |
| Var:B93 | YVTKTGIKI | 39 |
| Var:B94 | YVTKTNIKI | 40 |
| Var:B95 | YVTKTDIKI | 39 |
| Var:B96 | YVTKTEIKI | 39 |
| Var:B97 | YVTKTQIKI | 39 |
| Var:B98 | YVTKTSVKI | 42 |
| Var:B99 | YVTKTSIKV | 42 |
| Var:B100 | YVTKTSIKF | 39 |
| Var:B101 | YVTETKIKI | 35 |

TABLE 4.B.v

Suitable less immunogenic variants of RANKL
agretope 15 (residues 212-220; VGGFFKLRS);
B (wt) = 46.

| | | |
|---|---|---|
| | Variant | |
| Variant | sequence | B (alt) |

| | | |
|---|---|---|
| Var:B102 | VSGFFKLRS | 40 |
| Var:B103 | VGGYFKLRS | 43 |
| Var:B104 | VGGFFELRS | 42 |
| Var:B105 | VGGFFQLRS | 42 |
| Var:B106 | VGGFFKVRS | 43 |
| Var:B107 | VGGFFKLRT | 43 |
| Var:B108 | VGGFFKLRG | 42 |
| Var:B109 | VGGFFKLRN | 43 |
| Var:B110 | VGGFFKLRD | 42 |
| Var:B111 | VGGFFKLRE | 42 |
| Var:B112 | VGGFFKLRK | 42 |
| Var:B113 | VAGEEKIRS | 38 |
| Var:B114 | VAGFFKLRA | 37 |
| Var:B115 | VGAFFKIRS | 38 |
| Var:B116 | VGAFFKLRA | 37 |
| Var:B117 | VGGWFRLRS | 38 |
| Var:B118 | VGGWFKIRS | 39 |
| Var:B119 | VGGWFKLRA | 38 |
| Var:B120 | VGGFFSIRS | 39 |
| Var:B121 | VGGFFSFRS | 37 |
| Var:B122 | VGGFFSLRQ | 37 |
| Var:B123 | VGGFFNIRS | 39 |
| Var:B124 | VGGFFNFRS | 37 |
| Var:B125 | VGGFFKMRA | 41 |
| Var:B126 | VGGFFKIRA | 41 |
| Var:B127 | VGGFFKIRQ | 40 |
| Var:B128 | VGGFFKFRA | 39 |

TABLE 4.B.vi

Suitable less immunogenic variants of RANKL
agretope 17 (residues 212-220; ISIEVSNPS);
B (wt) = 42.

| | | |
|---|---|---|
| | Variant | |
| Variant | sequence | B (alt) |

| | | |
|---|---|---|
| Var:B129 | IDIEVSNPS | 38 |
| Var:B130 | ISLEVSNPS | 40 |
| Var:B131 | ISVEVSNPS | 41 |
| Var:B132 | ISFEVSNPS | 38 |
| Var:B133 | ISISVSNPS | 37 |
| Var:B134 | ISINVSNPS | 37 |
| Var:B135 | ISIQVSNPS | 39 |
| Var:B136 | ISIKVSNPS | 38 |
| Var:B137 | ISIEVANPS | 39 |
| Var:B138 | ISIEVGNPS | 38 |
| Var:B139 | ISIEVDNPS | 38 |
| Var:B140 | ISIEVENPS | 38 |
| Var:B141 | ISIEVQNPS | 38 |
| Var:B142 | ISIEVKNPS | 38 |
| Var:B143 | ISIEVSSPS | 37 |
| Var:B144 | ISIEVSTPS | 36 |
| Var:B145 | ISIEVSGPS | 36 |
| Var:B146 | ISIEVSDPS | 37 |
| Var:B147 | ISIEVSQPS | 36 |
| Var:B148 | ISIEVSHPS | 37 |
| Var:B149 | ISIEVSRPS | 36 |
| Var:B150 | ISIEVSKPS | 36 |
| Var:B151 | ISIEVSNPT | 39 |
| Var:B152 | ISIEVSNPA | 39 |
| Var:B153 | ISIEVSNPG | 38 |
| Var:B154 | ISIEVSNPN | 39 |
| Var:B155 | ISIEVSNPD | 38 |
| Var:B156 | ISIEVSNPE | 38 |
| Var:B157 | ISIEVSNPK | 38 |

TABLE 4.C.i

Suitable less immunogenic variants of APRIL
agretope 5 (residues 142-150; LRRGRGLQA);
B (wt) = 44.

| | | |
|---|---|---|
| | Variant | |
| Variant | sequence | B (alt) |

| | | |
|---|---|---|
| Var:C1 | LNRGRGLQA | 39 |
| Var:C2 | LERGRGLQA | 39 |
| Var:C3 | LQRGRGLQA | 40 |
| Var:C4 | LHRGRGLQA | 39 |
| Var:C5 | LKRGRGLQA | 41 |
| Var:C6 | LREGRGLQA | 39 |
| Var:C7 | LRQGRGLQA | 40 |
| Var:C8 | LRHGRGLQA | 39 |
| Var:C9 | LRKGRGLQA | 41 |
| Var:C10 | LRRSRGLQA | 38 |
| Var:C11 | LRRGRGFQA | 40 |
| Var:C12 | LRRGRGLQS | 41 |
| Var:C13 | LRRGRGLQG | 40 |
| Var:C14 | LRNGRGMQA | 37 |
| Var:C15 | LRNGRGIQA | 37 |
| Var:C16 | LRNGRGVQA | 36 |
| Var:C17 | LRRGRSIQA | 36 |
| Var:C18 | LRRGRAMQA | 36 |
| Var:C19 | LRRGRGMQT | 38 |
| Var:C20 | LRRGRGIQT | 38 |
| Var:C21 | LRRGRGVQT | 37 |

TABLE 4.C.II

Suitable less immunogenic variants of APRIL
agretope 9 (residues 164-172; LLYSQVLFQ);
B (wt) = 43.

| | | |
|---|---|---|
| | Variant | |
| Variant | sequence | B (alt) |

| | | |
|---|---|---|
| Var:C22 | LLHSQVLFQ | 38 |
| Var:C23 | LLWSQVLFQ | 38 |
| Var:C24 | LLYGQVLFQ | 39 |
| Var:C25 | LLYSQALFQ | 40 |
| Var:C26 | LLYSQMLFQ | 40 |
| Var:C27 | LLYSQILFQ | 42 |
| Var:C28 | LLYSQLLFQ | 40 |
| Var:C29 | LLYSQVIFQ | 41 |
| Var:C30 | LLYSQVVFQ | 40 |
| Var:C31 | LLYSQVFFQ | 39 |
| Var:C32 | LLYSQVLFN | 38 |
| Var:C33 | LLYSQVLFD | 38 |
| Var:C34 | LLYSQVLFE | 40 |
| Var:C35 | LLYSQVLFH | 38 |
| Var:C36 | LLYSQVLFR | 39 |
| Var:C37 | LLYSQVLFK | 39 |
| Var:C38 | LLYTQVLFM | 35 |

TABLE 4.C.iii

Suitable less immunogenic variants of APRIL
agretope 10 (residues 170-178; LFQDVTFTM);
B (wt) = 46.

| | | |
|---|---|---|
| | Variant | |
| Variant | sequence | B (alt) |

| | | |
|---|---|---|
| Var:C39 | FFQDVTFTM | 42 |
| Var:C40 | LWQDVTFTM | 41 |
| Var:C41 | LFDDVTFTM | 41 |
| Var:C42 | LFEDVTFTM | 43 |
| Var:C43 | LFQSVTFTM | 40 |
| Var:C44 | LFQNVTFTM | 41 |
| Var:C45 | LFQEVTFTM | 42 |
| Var:C46 | LFQQVTFTM | 40 |
| Var:C47 | LFQDVSFTM | 42 |
| Var:C48 | LFQDVAFTM | 41 |
| Var:C49 | LFQDVNFTM | 41 |
| Var:C50 | LFQDVTWTM | 41 |
| Var:C51 | LFQDVTFTQ | 42 |
| Var:C52 | LFQDVTFTL | 43 |
| Var:C53 | LFQDVTFTV | 42 |
| Var:C54 | LFQDVTYTI | 39 |

TABLE 4.C.iv

Suitable less immunogenic variants of APRIL
agretope 11 (residues 194-202; FRCIRSMPS);
B (wt) = 49.

| | | |
|---|---|---|
| | Variant | |
| Variant | sequence | B (alt) |

| | | |
|---|---|---|
| Var:C55 | FNCIRGMPS | 40 |
| Var:C56 | FNCIRDMPS | 40 |
| Var:C57 | FNCIREMPS | 40 |
| Var:C58 | FNCIRSMPT | 41 |
| Var:C59 | FNCIRSMPG | 40 |
| Var:C60 | FNCIRSMPK | 40 |
| Var:C61 | FECVRSMPS | 43 |
| Var:C62 | FECIRAMPS | 41 |
| Var:C63 | FECIRGMPS | 40 |
| Var:C64 | FECIRDMPS | 40 |
| Var:C65 | FECIREMPS | 40 |
| Var:C66 | FECIRQMPS | 40 |
| Var:C67 | FECIRSQPS | 40 |
| Var:C68 | FECIRSIPS | 40 |
| Var:C69 | FECIRSLPS | 41 |
| Var:C70 | FECIRSVPS | 40 |
| Var:C71 | FECIRSFPS | 40 |
| Var:C72 | FECIRSMPT | 41 |
| Var:C73 | FECIRSMPA | 41 |
| Var:C74 | FECIRSMPG | 40 |
| Var:C75 | FECIRSMPN | 41 |
| Var:C76 | FECIRSMPD | 40 |
| Var:C77 | FECIRSMPK | 40 |
| Var:C78 | FQCIRDMPS | 41 |
| Var:C79 | FQCIREMPS | 41 |
| Var:C80 | FQCIRSMPT | 42 |
| Var:C81 | FHCIRGMPS | 40 |
| Var:C82 | FHCIRDMPS | 40 |
| Var:C83 | FHCIREMPS | 40 |
| Var:C84 | FHCIRSMPT | 41 |
| Var:C85 | FHCIRSMPG | 40 |
| Var:C86 | FHCIRSMPK | 40 |
| Var:C87 | FKCIRGMPS | 42 |
| Var:C88 | FKCIRDMPS | 42 |
| Var:C89 | FKCIREMPS | 42 |
| Var:C90 | FKCIRSMPT | 43 |
| Var:C91 | FRCVRDMPS | 44 |
| Var:C92 | FRCVREMPS | 44 |
| Var:C93 | FRCIRGMPG | 41 |
| Var:C94 | FRCIRGMPN | 42 |
| Var:C95 | FRCIRGMPD | 41 |
| Var:C96 | FRCIRGMPE | 41 |
| Var:C97 | FRCIRDQPS | 41 |
| Var:C98 | FRCIRDIPS | 41 |
| Var:C99 | FRCIRDLPS | 42 |
| Var:C100 | FRCIRDVPS | 41 |
| Var:C101 | FRCIRDFPS | 41 |
| Var:C102 | FRCIRDMPT | 42 |
| Var:C103 | FRCIRDMPA | 42 |
| Var:C104 | FRCIRDMPG | 41 |
| Var:C105 | FRCIRDMPN | 42 |
| Var:C106 | FRCIRDMPD | 41 |
| Var:C107 | FRCIRDMPK | 41 |
| Var:C108 | FRCIREQPS | 41 |
| Var:C109 | FRCIREIPS | 41 |
| Var:C110 | FRCIREVPS | 41 |
| Var:C111 | FRCIREEPS | 41 |
| Var:C112 | FRCIREMPT | 42 |
| Var:C113 | FRCIREMPA | 42 |
| Var:C114 | FRCIREMPG | 41 |
| Var:C115 | FRCIREMPN | 42 |
| Var:C116 | FRCIREMPD | 41 |
| Var:C117 | FRCIREMPE | 41 |
| Var:C118 | FRCIREMPK | 41 |
| Var:C119 | FRCIRQQPS | 41 |
| Var:C120 | FRCIRQVPS | 41 |
| Var:C121 | FRCIRQFPS | 41 |
| Var:C122 | FRCIRQMPT | 42 |
| Var:C123 | FRCIRQMPG | 41 |
| Var:C124 | FRCIRQMPN | 42 |
| Var:C125 | FRCIRQMPD | 41 |
| Var:C126 | FRCIRQMPE | 41 |
| Var:C127 | FNCVRAMPS | 40 |
| Var:C128 | FNCVRSLPS | 40 |
| Var:C129 | FNCVRSMPA | 40 |
| Var:C130 | FNCVRSMPN | 40 |
| Var:C131 | FQCVRGMPS | 40 |
| Var:C132 | FQCVRQMPS | 40 |
| Var:C133 | FQCVRSQPS | 40 |
| Var:C134 | FQCVRSVPS | 40 |
| Var:C135 | FQCVRSFPS | 40 |
| Var:C136 | FQCVRSMPG | 40 |
| Var:C137 | FQCVRSMPN | 41 |
| Var:C138 | FQCVRSMPK | 40 |
| Var:C139 | FHCVRAMPS | 40 |
| Var:C140 | FHCVRSLPS | 40 |
| Var:C141 | FHCVRSMPA | 40 |
| Var:C142 | FHCVRSMPN | 40 |
| Var:C143 | FKCVRQMPS | 41 |
| Var:C144 | FKCVRSQPS | 41 |
| Var:C145 | FKCVRSIPS | 41 |
| Var:C146 | FKCVRSVPS | 41 |
| Var:C147 | FKCVRSFPS | 41 |
| Var:C148 | FKCVRSMPA | 42 |
| Var:C149 | FKCVRSMPG | 41 |
| Var:C150 | FKCVRSMPN | 42 |
| Var:C151 | FKCVRSMPK | 41 |
| Var:C152 | FKCIRAMPA | 40 |
| Var:C153 | FKCIRAMPN | 40 |
| Var:C154 | FRCVRAQPS | 41 |
| Var:C155 | FRCVRAMPT | 42 |
| Var:C156 | FRCVRAMPG | 41 |
| Var:C157 | FRCVRAMPN | 42 |
| Var:C158 | FRCVRGQPS | 40 |
| Var:C159 | FRCVRGFPS | 40 |
| Var:C160 | FRCVRGMPT | 41 |
| Var:C161 | FRCVRGMPA | 41 |
| Var:C162 | FRCVRGMPK | 40 |
| Var:C163 | FRCVRQMPK | 40 |
| Var:C164 | FRCVRSQPT | 41 |
| Var:C165 | FRCVRSQPG | 40 |
| Var:C166 | FRCVRSQPK | 40 |
| Var:C167 | FRCVRSIPT | 41 |
| Var:C168 | FRCVRSIPG | 40 |
| Var:C169 | FRCVRSVPT | 41 |
| Var:C170 | FRCVRSVPG | 40 |
| Var:C171 | FRCVRSVPK | 40 |
| Var:C172 | FRCVRSFPT | 41 |
| Var:C173 | FRCVRSFPG | 40 |
| Var:C174 | FRCVRSFPK | 40 |

TABLE 4.D.i
Suitable less immunogenic variants of CD40L
agretope 12 (residues 84-92; LRAANTHSS);
B (wt) = 44.

| | | |
|---|---|---|
| | Variant | |
| Variant | sequence | B (alt) |

| | | |
|---|---|---|
| Var:D1 | LEAANTHSS | 39 |
| Var:D2 | LRAANAHSS | 39 |
| Var:D3 | LRAANTHST | 41 |
| Var:D4 | LRAANTHSG | 40 |
| Var:D5 | LRAANTHSN | 41 |
| Var:D6 | LRAANTHSD | 40 |
| Var:D7 | LRAANTHSE | 40 |
| Var:D8 | FRAGNTHSS | 36 |
| Var:D9 | LNASNTHSS | 36 |
| Var:D10 | LNAANTHSA | 36 |
| Var:D11 | LQASNTHSS | 37 |
| Var:D12 | LQATNTHSS | 36 |
| Var:D13 | LQAGNTHSS | 36 |
| Var:D14 | LQAANSHSS | 36 |
| Var:D15 | LQAANTHSA | 37 |
| Var:D16 | LQAANTHSK | 36 |
| Var:D17 | LHASNTHSS | 36 |
| Var:D18 | LHAANTHSA | 36 |
| Var:D19 | LKASNTHSS | 38 |
| Var:D20 | LKATNTHSS | 37 |
| Var:D21 | LKAGNTHSS | 37 |
| Var:D22 | LKAANSHSS | 37 |
| Var:D23 | LKAANNHSS | 36 |
| Var:D24 | LKAANVHSS | 36 |
| Var:D25 | LKAANTHSA | 38 |
| Var:D26 | LKAANTHSK | 37 |
| Var:D27 | LRATNTHSK | 36 |
| Var:D28 | LRAGNSHSS | 36 |
| Var:D29 | LRAGNTHSK | 36 |
| Var:D30 | LRAANSHSA | 37 |
| Var:D31 | LRAANSHSK | 36 |

TABLE 4.E.i

Suitable less immunogenic variants of TRAIL
agretope 2 (residues 174-182; LVIHEKGFY);
B (wt) = 49.

| | | |
|---|---|---|
| | Variant | |
| Variant | sequence | B (alt) |

| | | |
|---|---|---|
| Var:E1 | LTIHEKGFY | 46 |
| Var:E2 | LAIHEKGFY | 46 |
| Var:E3 | LMIHEKGFY | 46 |
| Var:E4 | LIIHEKGFY | 48 |
| Var:E5 | LLIHEKGFY | 46 |
| Var:E6 | LVVHEKGFY | 48 |
| Var:E7 | LVIEEKGFY | 41 |
| Var:E8 | LVIHESGFY | 44 |
| Var:E9 | LVIHENGFY | 44 |
| Var:E10 | LVIHEEGFY | 45 |
| Var:E11 | LVIHEQGFY | 45 |
| Var:E12 | LVLHEKGFW | 42 |
| Var:E13 | LVFHERGFY | 42 |
| Var:E14 | LVFHEKGFW | 40 |
| Var:E15 | LVIHERGFW | 41 |

TABLE 4.E.ii

Suitable less immunogenic variants of TRAIL
agretope 7 (residues 207-215; VQYIYKYTS);
B (wt) = 48.

| | | |
|---|---|---|
| | Variant | |
| Variant | sequence | B (alt) |

| | | |
|---|---|---|
| Var:E16 | VSYIYKYTS | 43 |
| Var:E17 | VDYIYKYTS | 43 |
| Var:E18 | VEYIYKYTS | 45 |
| Var:E19 | VQHIYKYTS | 43 |
| Var:E20 | VQWIYKYTS | 43 |
| Var:E21 | VQYIYEYTS | 44 |
| Var:E22 | VQYIYQYTS | 44 |
| Var:E23 | VQYIYKYTT | 45 |
| Var:E24 | VQYIYKYTA | 45 |
| Var:E25 | VQYIYKYTG | 44 |
| Var:E26 | VQYIYKYTN | 45 |
| Var:E27 | VQYIYKYTD | 44 |
| Var:E28 | VQYIYKYTE | 44 |
| Var:E29 | VQYIYKYTK | 44 |
| Var:E30 | VNYVYKYTS | 42 |
| Var:E31 | VNYIYRYTS | 40 |
| Var:E32 | VHYVYKYTS | 42 |
| Var:E33 | VHYIYRYTS | 40 |
| Var:E34 | VQYVYRYTS | 44 |
| Var:E35 | VQYVYKYTQ | 43 |

TABLE 4.E.iii

Suitable less immunogenic variants of TRAIL
agretope 10 (residues 221-229; LLMKSARNS);
B (wt) = 41.

| | | |
|---|---|---|
| | Variant | |
| Variant | sequence | B (alt) |

| | | |
|---|---|---|
| Var:E36 | LLMESARNS | 37 |
| Var:E37 | LLMKSAENS | 36 |
| Var:E38 | LLMKSARNT | 38 |
| Var:E39 | LLMKSARNN | 38 |
| Var:E40 | LFQKSARNS | 33 |
| Var:E41 | LFMKSARND | 33 |
| Var:E42 | LLQQSARNS | 33 |
| Var:E43 | LLQKSGRNS | 33 |
| Var:E44 | LLQKSAQNS | 33 |
| Var:E45 | LLQKSAKNS | 34 |
| Var:E46 | LLQKSARNA | 34 |
| Var:E47 | LLQKSARNG | 33 |
| Var:E48 | LLQKSARND | 33 |
| Var:E49 | LLQKSARNE | 33 |
| Var:E50 | LLQKSARNQ | 33 |
| Var:E51 | LLQKSARNK | 33 |
| Var:E52 | LLLSSARNS | 33 |
| Var:E53 | LLLKSANNS | 33 |
| Var:E54 | LLLKSAKNS | 35 |
| Var:E55 | LLLKSARND | 34 |
| Var:E56 | LLVKSGRNS | 33 |
| Var:E57 | LLVKSAQNS | 33 |
| Var:E58 | LLVKSAKNS | 34 |
| Var:E59 | LLVKSARNA | 34 |
| Var:E60 | LLVKSARNG | 33 |
| Var:E61 | LLVKSARND | 33 |
| Var:E62 | LLVKSARNE | 33 |
| Var:E63 | LLVKSARNK | 33 |
| Var:E64 | LLFKSAQNS | 33 |
| Var:E65 | LLFKSAKNS | 34 |
| Var:E66 | LLFKSARNG | 33 |
| Var:E67 | LLFKSARND | 33 |
| Var:E68 | LLFKSARNE | 33 |
| Var:E69 | LLFKSARNK | 33 |
| Var:E70 | LLMSSAKNS | 33 |
| Var:E71 | LLMSSARNA | 33 |
| Var:E72 | LLMNSAKNS | 33 |
| Var:E73 | LLMQSAQNS | 33 |
| Var:E74 | LLMQSARND | 33 |
| Var:E75 | LLMQSARNE | 33 |
| Var:E76 | LLMKSSRND | 34 |
| Var:E77 | LLMKSGQNS | 33 |
| Var:E78 | LLMKSGKNS | 34 |
| Var:E79 | LLMKSGRNG | 33 |
| Var:E80 | LLMKSGRND | 33 |
| Var:E81 | LLMKSGRNE | 33 |
| Var:E82 | LLMKSGRNK | 33 |
| Var:E83 | LLMKSANNA | 33 |
| Var:E84 | LLMKSAQNA | 34 |
| Var:E85 | LLMKSAQNG | 33 |
| Var:E86 | LLMKSAQND | 33 |
| Var:E87 | LLMKSAQNE | 33 |
| Var:E88 | LLMKSAQNK | 33 |
| Var:E89 | LLMKSAHNA | 33 |
| Var:E90 | LLMKSAKNA | 35 |
| Var:E91 | LLMKSAKNG | 34 |
| Var:E92 | LLMKSAKND | 34 |
| Var:E93 | LLMKSAKNK | 34 |

TABLE 4.E.iv

Suitable less immunogenic variants of TRAIL
agretope 14 (residues 256-264; IFVSVTNEH);
B (wt) = 46.

| | | |
|---|---|---|
| | Variant | |
| Variant | sequence | B (alt) |

| | | |
|---|---|---|
| Var:E94 | IWVSVTNEH | 41 |
| Var:E95 | IFTSVTNEH | 43 |
| Var:E96 | IFASVTNEH | 43 |
| Var:E97 | IFVTVTNEH | 43 |
| Var:E98 | IFVGVTNEH | 42 |
| Var:E99 | IFVSVSNEH | 42 |
| Var:E100 | IFVSVANEH | 41 |
| Var:E101 | IFVSVNNEH | 41 |
| Var:E102 | IFVSWNEH | 41 |
| Var:E103 | IFVSVTSEH | 41 |
| Var:E104 | IFVSVTTEH | 40 |
| Var:E105 | IFVSVTGEH | 40 |
| Var:E106 | IFVSVTDEH | 41 |
| Var:E107 | IFVSVTEEH | 40 |
| Var:E108 | IFVSVTQEH | 40 |
| Var:E109 | IFVSVTHEH | 41 |
| Var:E110 | IFVSVTKEH | 40 |
| Var:E111 | IFVSVTNEN | 39 |
| Var:E112 | IFVSVTNEE | 38 |
| Var:E113 | IFVSVTNER | 38 |
| Var:E114 | IFVSVTNEY | 40 |

TABLE 4.E.v

Suitable less immunogenic variants of TRAIL
agretope 15 (residues 257-265; FVSVTNEHL);
B (wt) = 46.

| | | |
|---|---|---|
| | Variant | |
| Variant | sequence | B (alt) |

| | | |
|---|---|---|
| Var:E115 | MVSVTNEHL | 40 |
| Var:E116 | IVSVTNEHL | 40 |
| Var:E117 | LVSVTNEHL | 40 |
| Var:E118 | FTSVTNEHL | 43 |
| Var:E119 | FASVTNEHL | 43 |
| Var:E120 | FMSVTNEHL | 43 |
| Var:E121 | FISVTNEHL | 45 |
| Var:E122 | FLSVTNEHL | 43 |
| Var:E123 | FVDVTNEHL | 42 |
| Var:E124 | FVEVTNEHL | 42 |
| Var:E125 | FVSATNEHL | 43 |
| Var:E126 | FVSVTDEHL | 41 |
| Var:E127 | FVSVTEEHL | 40 |
| Var:E128 | FVSVTQEHL | 40 |
| Var:E129 | FVSVTREHL | 40 |
| Var:E130 | FVSVTKEHL | 40 |
| Var:E131 | FVSVTNDHL | 43 |
| Var:E132 | FVSVTNKHL | 42 |
| Var:E133 | FVSVTNEHV | 43 |
| Var:E134 | FVSVTNEHF | 42 |
| Var:E135 | FVTLTNEHL | 40 |
| Var:E136 | FVTVTHEHL | 38 |
| Var:E137 | FVTVTNRHL | 38 |
| Var:E138 | FVTVTNEHM | 41 |
| Var:E139 | FVALTNEHL | 40 |
| Var:E140 | FVAVTHEHL | 38 |
| Var:E141 | FVAVTNRHL | 38 |
| Var:E142 | FVAVTNEHM | 41 |
| Var:E143 | FVQLTNEHL | 39 |
| Var:E144 | FVQVTHEHL | 37 |
| Var:E145 | FVQVTNRHL | 37 |
| Var:E146 | FVQVTNEHM | 40 |
| Var:E147 | FVKLTNEHL | 39 |
| Var:E148 | FVKVTHEHL | 37 |
| Var:E149 | FVKVTNRHL | 37 |
| Var:E150 | FVKVTNEHM | 40 |
| Var:E151 | FVSMTHEHL | 38 |
| Var:E152 | FVSLTHEHL | 38 |
| Var:E153 | FVSVTGEHM | 38 |
| Var:E154 | FVSVTHEHM | 39 |
| Var:E155 | FVSVTNRHM | 39 |

### Example 3

### Identification of Suitable Less Immunogenic Sequences for MHC-Binding Agretopes as Determined by PDA® Technology

Table 5. Each position in the agretopes of interest was analyzed to identify a subset of amino acid substitutions that are potentially compatible with maintaining the structure and function of the protein. PDA® technology calculations were run for each position of each nine-mer agretope and compatible amino acids for each position were saved. In these calculations, side-chains within 5 Angstroms of the position of interest were permitted to change conformation but not amino acid identity. The variant agretopes were then analyzed for immunogenicity. The PDA® energies and Iscore values for the wild-type nine-mer agretope were compared to the variants and the subset of variant sequences with lower predicted immunogenicity and PDA® energies within 5.0 kcal/mol of the wild-type were noted. In the tables below, E(PDA) is the energy determined using PDA® technology calculations compared against the wild-type, Iscore: Anchor is the Iscore for the agretope, and Iscore: Overlap is the sum of the Iscores for all of the overlapping agretopes.

TABLE 5.A.i
Less immunogenic variants of BAFF agretope 2.

| | | Iscore | Iscore |
|---|---|---|---|
| Var. | E(PDA) | Anchor | Overlap |
| wt | 0.00 | 24.2 | 17.1 |
| L169A | −0.70 | 11.3 | 0.0 |
| L169D | 1.11 | 2.2 | 0.0 |
| L169E | 0.99 | 15.9 | 0.0 |
| L169F | 0.76 | 23.3 | 4.0 |
| L169G | 0.65 | 19.9 | 0.0 |
| L169H | −0.44 | 23.3 | 0.0 |
| L169N | −0.12 | 23.3 | 0.0 |
| L169S | −0.06 | 5.8 | 0.0 |
| L169T | 0.17 | 11.3 | 0.0 |
| L169W | 3.28 | 11.3 | 4.0 |
| L169Y | 0.81 | 23.3 | 4.0 |
| L170A | 3.78 | 11.3 | 3.5 |
| L170D | 4.20 | 2.2 | 2.8 |
| L170E | 3.18 | 2.2 | 8.4 |
| L170G | 4.54 | 16.4 | 8.4 |
| L170P | 2.77 | 16.4 | 3.5 |
| L170S | 4.17 | 16.4 | 7.1 |
| L170T | 4.16 | 11.3 | 15.7 |
| S171G | 3.53 | 13.6 | 14.3 |
| K173N | 3.80 | 14.7 | 17.1 |
| R174D | 2.51 | 5.2 | 2.8 |
| R174E | 1.77 | 16.8 | 2.8 |
| R174G | 4.31 | 18.6 | 8.4 |
| R174H | 2.55 | 19.7 | 2.8 |
| R174K | 0.07 | 17.7 | 3.9 |
| R174Q | 1.89 | 19.4 | 2.8 |
| R174S | 2.61 | 16.8 | 10.2 |
| R174T | 2.32 | 16.8 | 6.7 |
| R174Y | 3.32 | 22.4 | 8.4 |
| S176A | 0.57 | 14.4 | 17.1 |
| S176D | 0.94 | 6.6 | 17.1 |
| S176E | 0.84 | 6.6 | 17.1 |
| S176F | 1.25 | 21.8 | 17.1 |
| S176G | 0.74 | 13.3 | 17.1 |
| S176H | 1.18 | 9.9 | 17.1 |
| S176K | 1.21 | 5.8 | 17.1 |
| S176M | 2.12 | 22.0 | 17.1 |
| S176N | 0.48 | 5.5 | 17.1 |
| S176P | 2.25 | 5.5 | 17.1 |
| S176Q | 1.22 | 13.7 | 17.1 |
| S176R | 1.15 | 13.3 | 17.1 |
| S176T | 0.34 | 5.5 | 17.1 |
| S176V | 1.15 | 18.2 | 17.1 |
| S176W | 3.15 | 2.2 | 17.1 |
| S176Y | 1.20 | 13.3 | 17.1 |

TABLE 5.A.ii
Less immunogenic variants of BAFF agretope 6.

| | | Iscore | Iscore |
|---|---|---|---|
| Var. | E(PDA) | Anchor | Overlap |
| wt | 0.00 | 42.6 | 32.7 |
| Y192D | 2.99 | 0.0 | 31.5 |
| Y192E | 1.50 | 0.0 | 31.5 |
| Y192K | 4.04 | 0.0 | 31.5 |
| Y192Q | 3.25 | 0.0 | 31.5 |
| F194H | 4.45 | 20.9 | 31.0 |
| I195D | 4.69 | 0.0 | 28.6 |
| I195L | 2.08 | 17.8 | 29.9 |
| I195N | 3.77 | 7.6 | 28.6 |
| I195T | 3.67 | 32.6 | 28.6 |
| L200E | 3.31 | 0.0 | 6.1 |
| L200K | 3.96 | 0.0 | 6.1 |
| L200N | 4.29 | 3.0 | 6.1 |
| L200Q | 3.46 | 0.0 | 6.1 |

TABLE 5.A.iii
Less immunogenic variants of BAFF agretope 9.

| | | Iscore | Iscore |
|---|---|---|---|
| Var. | E(PDA) | Anchor | Overlap |
| wt | 0.00 | 26.2 | 46.7 |
| L200E | 3.31 | 0.0 | 3.6 |
| L200K | 3.96 | 0.0 | 3.6 |
| L200N | 4.29 | 0.0 | 6.6 |
| L200Q | 3.46 | 0.0 | 3.6 |
| D203A | 4.22 | 2.5 | 46.7 |
| T205D | −3.46 | 0.0 | 46.7 |
| T205E | −4.94 | 0.0 | 46.7 |
| T205G | −2.83 | 5.2 | 46.7 |
| T205N | −5.81 | 5.2 | 46.7 |
| T205Q | −6.91 | 13.1 | 46.7 |
| T205S | −3.93 | 13.1 | 46.7 |
| Y206D | −0.04 | 15.8 | 46.7 |
| Y206H | 0.05 | 19.8 | 46.7 |
| Y206K | −1.35 | 15.8 | 46.7 |
| Y206R | −0.71 | 13.1 | 46.7 |
| Y206W | −0.75 | 13.1 | 46.7 |
| M208A | −0.15 | 13.1 | 46.7 |
| M208E | 4.79 | 5.2 | 46.7 |
| M208G | 2.51 | 13.1 | 46.7 |
| M208K | 4.37 | 5.2 | 46.7 |
| M208R | 4.25 | 13.1 | 46.7 |
| M208T | 1.85 | 5.2 | 46.7 |

TABLE 5.A.iv
Less immunogenic variants of BAFF agretope 10.

| | | Iscore | Iscore |
|---|---|---|---|
| Var. | E(PDA) | Anchor | Overlap |
| wt | 0.00 | 26.4 | 5.2 |
| I212D | 4.99 | 0.0 | 5.2 |
| I212E | 3.42 | 0.0 | 5.2 |
| I212N | 4.17 | 0.0 | 5.2 |
| I212Q | 2.86 | 0.0 | 5.2 |
| I212T | 3.49 | 0.0 | 5.2 |
| Q213A | 4.44 | 8.4 | 5.2 |
| Q213D | 1.08 | 0.0 | 5.2 |
| Q213E | 0.70 | 8.4 | 5.2 |
| R214E | 3.57 | 2.6 | 5.2 |
| R214N | 3.20 | 26.0 | 5.2 |
| K215A | −1.05 | 18.2 | 5.2 |
| K215D | −0.04 | 26.2 | 5.2 |
| K215E | −2.16 | 5.7 | 5.2 |
| K215G | 0.27 | 5.2 | 5.2 |
| K215H | 0.36 | 25.2 | 5.2 |
| K215I | −2.09 | 17.8 | 5.2 |
| K215L | −2.87 | 24.8 | 5.2 |
| K215N | −0.94 | 22.3 | 5.2 |
| K215Q | −1.33 | 23.6 | 5.2 |
| K215R | −1.47 | 21.4 | 5.2 |
| K215T | −0.71 | 8.0 | 5.2 |
| K215V | −2.02 | 8.0 | 5.2 |
| K215W | 0.43 | 25.2 | 5.2 |
| V217D | 2.63 | 0.0 | 5.2 |
| V217E | 2.85 | 0.0 | 5.2 |
| V217I | 1.34 | 19.1 | 5.2 |
| V217K | 2.80 | 26.0 | 5.2 |
| V217N | 1.11 | 2.9 | 5.2 |
| V217Q | 0.40 | 8.4 | 5.2 |
| V217S | −0.49 | 8.8 | 5.2 |
| V217T | −0.27 | 20.5 | 5.2 |
| V217Y | 2.10 | 0.0 | 5.2 |
| H218D | −0.78 | 2.6 | 5.2 |
| H218E | 0.48 | 8.4 | 5.2 |
| H218G | −1.07 | 5.6 | 5.2 |
| H218K | 0.42 | 26.0 | 5.2 |
| H218N | −0.65 | 24.9 | 5.2 |
| H218Q | 0.28 | 20.0 | 5.2 |
| H218S | −0.85 | 9.4 | 5.2 |
| H218T | −0.06 | 8.4 | 5.2 |
| F220A | −1.88 | 16.5 | 0.0 |
| F220D | −0.60 | 12.8 | 0.0 |
| F220E | −0.61 | 14.6 | 0.0 |
| F220G | −1.58 | 21.4 | 5.2 |
| F220H | −0.44 | 16.9 | 5.2 |
| F220N | −0.98 | 13.3 | 5.2 |
| F220P | 1.46 | 9.3 | 0.0 |
| F220T | −1.38 | 12.0 | 0.0 |
| F220W | 2.11 | 6.9 | 0.0 |

TABLE 5.A.v
Less immunogenic variants of BAFF agretope 12.

| | | Iscore | Iscore |
|---|---|---|---|
| Var. | E(PDA) | Anchor | Overlap |
| wt | 0.00 | 28.0 | 27.4 |
| L226A | 2.43 | 0.0 | 27.4 |
| L226D | 2.01 | 0.0 | 27.4 |
| L226E | 0.92 | 0.0 | 27.4 |
| L226F | 3.02 | 27.0 | 27.4 |
| L226G | 3.17 | 0.0 | 27.4 |
| L226H | 2.88 | 0.0 | 27.4 |
| L226K | 2.74 | 0.0 | 27.4 |
| L226N | 1.94 | 0.0 | 27.4 |
| L226P | 4.55 | 0.0 | 27.4 |
| L226Q | 2.25 | 0.0 | 27.4 |
| L226R | 2.93 | 0.0 | 27.4 |
| L226S | 2.55 | 0.0 | 27.4 |
| L226T | 1.73 | 0.0 | 27.4 |
| L226Y | 2.59 | 27.0 | 27.4 |
| V227A | 2.29 | 1.1 | 21.9 |
| V227D | 0.44 | 0.0 | 21.9 |
| V227E | 0.23 | 1.1 | 16.6 |
| V227H | 2.71 | 6.8 | 17.7 |
| V227K | −0.25 | 13.0 | 17.1 |
| V227L | −0.14 | 11.4 | 27.4 |
| V227N | 1.76 | 6.8 | 16.6 |
| V227Q | 0.84 | 13.0 | 16.6 |
| V227T | 0.88 | 1.1 | 16.6 |
| V227W | 1.72 | 1.1 | 17.9 |
| L229A | 4.89 | 14.0 | 21.9 |
| L229D | 4.85 | 26.2 | 21.9 |
| L229E | 3.47 | 0.4 | 21.9 |
| L229K | 4.71 | 20.3 | 21.9 |
| L229N | 3.60 | 14.0 | 23.3 |
| L229Q | 3.28 | 13.2 | 21.9 |
| L229T | 4.16 | 5.8 | 26.2 |
| R231A | 0.46 | 20.4 | 13.0 |
| R231D | 2.68 | 1.4 | 11.2 |
| R231E | 0.90 | 0.0 | 13.0 |
| R231G | 2.30 | 7.0 | 13.3 |
| R231L | −0.52 | 16.9 | 17.1 |
| R231M | −0.57 | 2.5 | 17.1 |
| R231Q | 0.22 | 8.0 | 17.1 |
| Q234A | 1.84 | 19.4 | 27.4 |
| Q234D | 3.47 | 4.2 | 27.4 |
| Q234E | 1.73 | 9.7 | 27.4 |
| Q234G | 3.86 | 17.5 | 27.4 |
| Q234K | −3.82 | 15.2 | 27.4 |
| Q234R | 1.11 | 19.6 | 27.4 |

TABLE 5.A.vi
Less immunogenic variants of BAFF agretope 16.

| | | Iscore | Iscore |
|---|---|---|---|
| Var. | E(PDA) | Anchor | Overlap |
| wt | 0.00 | 40.9 | 0.0 |
| V276A | 2.87 | 0.0 | 0.0 |
| V276N | 4.97 | 0.0 | 0.0 |
| V276S | 2.50 | 0.0 | 0.0 |
| V276T | 1.63 | 0.0 | 0.0 |
| T277S | 4.03 | 34.0 | 0.0 |
| F278H | 4.43 | 19.4 | 0.0 |
| F278K | 4.93 | 14.8 | 0.0 |
| F279H | 3.94 | 25.9 | 0.0 |
| F279Y | 1.94 | 28.7 | 0.0 |
| A281G | 3.12 | 34.9 | 0.0 |
| L282A | 4.64 | 6.5 | 0.0 |
| L282D | 4.80 | 0.0 | 0.0 |
| L282E | 3.72 | 0.0 | 0.0 |
| L282K | 3.66 | 0.0 | 0.0 |
| L282M | 2.38 | 34.0 | 0.0 |
| L282N | 3.87 | 15.2 | 0.0 |
| L282Q | 2.31 | 1.2 | 0.0 |
| L282T | 3.74 | 6.5 | 0.0 |
| L284E | 3.17 | 0.0 | 0.0 |
| L284M | 4.65 | 30.7 | 0.0 |
| L284N | 4.29 | 5.6 | 0.0 |
| L284Q | 4.69 | 7.3 | 0.0 |
| L284T | 4.74 | 14.8 | 0.0 |
| L284V | 3.80 | 29.6 | 0.0 |

TABLE 5.B.i
Less immunogenic variants of RANKL agretope 3.

| | | Iscore | Iscore |
|---|---|---|---|
| Var. | E(PDA) | Anchor | Overlap |
| wt | 0.00 | 28.0 | 48.2 |
| F213A | −0.17 | 0.0 | 41.3 |
| F213D | −0.35 | 0.0 | 40.1 |
| F213E | −0.91 | 0.0 | 40.1 |
| F213G | 1.97 | 0.0 | 40.1 |
| F213H | −3.87 | 0.0 | 41.3 |
| F213L | 2.83 | 16.9 | 48.2 |
| F213M | −3.20 | 16.9 | 41.3 |
| F213N | 0.20 | 0.0 | 41.3 |
| F213Q | −0.13 | 0.0 | 43.2 |
| F213S | 1.31 | 0.0 | 41.3 |
| F213T | −0.83 | 0.0 | 46.6 |
| Y214F | 2.40 | 25.9 | 48.2 |
| Y215H | 4.99 | 19.1 | 31.9 |
| L216D | 4.81 | 17.2 | 9.3 |
| L216E | 3.19 | 23.7 | 9.3 |
| L216N | 3.78 | 15.1 | 26.3 |
| L216Q | 3.97 | 15.7 | 21.4 |
| L216T | 4.00 | 11.2 | 16.0 |
| L216V | 1.70 | 8.6 | 44.6 |
| A218G | 2.76 | 16.8 | 42.7 |
| N219A | 4.55 | 25.5 | 45.6 |
| N219D | 4.46 | 3.4 | 21.4 |
| N219H | 0.97 | 26.4 | 23.9 |
| N219K | −1.38 | 24.7 | 39.3 |
| N219Q | 4.21 | 21.3 | 17.2 |
| N219T | 2.06 | 21.0 | 41.0 |

TABLE 5.B.ii
Less immunogenic variants of RANKL agretope 4.

| | | Iscore | Iscore |
|---|---|---|---|
| Var. | E(PDA) | Anchor | Overlap |
| wt | 0.00 | 34.6 | 57.0 |
| I220S | −5.55 | 8.5 | 51.6 |
| I220A | −4.58 | 5.5 | 54.6 |
| I220G | −2.89 | 3.7 | 51.6 |
| I220N | −2.07 | 19.1 | 51.9 |
| F222Y | −1.52 | 17.5 | 57.0 |
| N219K | −1.38 | 25.3 | 54.2 |
| I220L | −0.60 | 32.6 | 51.6 |
| I220E | −0.23 | 1.2 | 51.6 |
| I220D | 0.11 | 1.2 | 51.6 |
| I220M | 0.52 | 32.0 | 51.6 |
| I220Q | 0.61 | 1.2 | 51.6 |
| N219H | 0.97 | 8.8 | 56.9 |
| I220H | 1.15 | 17.7 | 51.6 |
| L216V | 1.70 | 16.0 | 52.7 |
| N219T | 2.06 | 27.4 | 50.1 |
| F222H | 2.91 | 20.9 | 41.5 |
| L216E | 3.19 | 1.2 | 47.3 |
| L216N | 3.78 | 16.0 | 40.9 |
| L216Q | 3.97 | 7.9 | 44.7 |
| L216T | 4.00 | 7.9 | 34.8 |
| N219Q | 4.21 | 3.7 | 50.3 |
| N219D | 4.46 | 7.9 | 32.5 |
| N219A | 4.55 | 32.0 | 54.6 |
| F222K | 4.56 | 8.6 | 45.4 |
| F222M | 4.59 | 33.4 | 50.2 |
| L216D | 4.81 | 1.2 | 40.8 |
| Y215H | 4.99 | 29.6 | 36.8 |

TABLE 5.B.iii
Less immunogenic variants of RANKL agretope 10.

| | | Iscore | Iscore |
|---|---|---|---|
| Var. | E(PDA) | Anchor | Overlap |
| wt | 0.00 | 44.6 | 68.7 |
| I247A | −3.98 | 17.6 | 34.5 |
| I247S | −3.84 | 41.5 | 30.3 |
| I247N | −3.78 | 5.2 | 43.9 |
| I247E | −3.67 | 5.2 | 38.2 |
| I247T | −3.62 | 5.2 | 38.2 |
| I247G | −3.47 | 13.1 | 26.2 |
| I247D | −3.24 | 5.2 | 23.0 |
| T245H | −3.02 | 42.1 | 64.0 |
| I247Q | −2.96 | 21.2 | 43.9 |
| I247H | −2.72 | 10.1 | 45.1 |
| I247R | −2.58 | 13.1 | 55.0 |
| I247K | −2.50 | 17.3 | 36.6 |
| I247V | −1.54 | 13.9 | 54.7 |
| I247Y | −1.34 | 13.1 | 61.2 |
| T245K | −0.15 | 39.0 | 64.0 |
| T245E | 0.12 | 28.3 | 64.0 |
| M239T | 0.59 | 0.0 | 54.2 |
| I247W | 0.63 | 0.0 | 65.8 |
| T245D | 0.64 | 13.1 | 64.0 |
| V240N | 1.10 | 5.2 | 64.6 |
| V240T | 1.19 | 0.0 | 57.5 |
| V242T | 1.19 | 19.1 | 36.1 |
| M239E | 1.24 | 0.0 | 54.2 |
| V240E | 1.50 | 0.0 | 47.1 |
| V242K | 1.80 | 24.6 | 44.1 |
| V240A | 1.85 | 0.0 | 67.9 |
| T245G | 1.86 | 40.4 | 64.0 |
| V240D | 1.89 | 0.0 | 47.1 |
| Y241H | 1.96 | 17.9 | 56.3 |
| V240S | 2.44 | 0.0 | 68.5 |
| V242E | 2.59 | 5.2 | 33.2 |
| M239K | 2.71 | 0.0 | 67.2 |
| M239N | 2.73 | 0.0 | 67.2 |
| M239A | 2.98 | 0.0 | 54.2 |
| V240K | 3.32 | 10.0 | 52.7 |
| Y241E | 3.38 | 0.0 | 27.4 |
| Y241T | 3.40 | 17.9 | 62.8 |
| M239S | 3.46 | 0.0 | 53.8 |
| Y241D | 3.48 | 0.0 | 51.4 |
| V242A | 3.59 | 38.4 | 36.1 |
| M239D | 3.61 | 0.0 | 53.4 |
| V242Q | 3.73 | 43.6 | 44.1 |
| V242N | 3.73 | 38.4 | 50.9 |
| Y241N | 3.85 | 26.4 | 63.3 |
| M239H | 3.86 | 0.0 | 67.2 |
| V240G | 4.25 | 0.0 | 68.5 |
| Y241A | 4.29 | 17.9 | 45.1 |
| Y241Q | 4.50 | 17.9 | 57.0 |
| V242D | 4.64 | 26.2 | 27.1 |

TABLE 5.B.iv
Less immunogenic variants of RANKL agretope 12.

| | | Iscore | Iscore |
|---|---|---|---|
| Var. | E(PDA) | Anchor | Overlap |
| wt | 0.00 | 35.4 | 77.9 |
| I247A | −3.98 | 11.4 | 40.7 |
| I247S | −3.84 | 7.2 | 64.6 |
| I247N | −3.78 | 20.9 | 28.3 |
| I247E | −3.67 | 15.2 | 28.3 |
| I247T | −3.62 | 15.2 | 28.3 |
| I247G | −3.47 | 3.1 | 36.1 |
| I249E | −3.25 | 6.7 | 67.6 |
| I247D | −3.24 | 0.0 | 28.3 |
| I249N | −3.03 | 6.3 | 67.6 |
| I249D | −3.03 | 6.3 | 67.6 |
| I247Q | −2.96 | 20.9 | 44.2 |
| I249L | −2.74 | 33.6 | 67.6 |
| I247H | −2.72 | 22.0 | 33.2 |
| I247R | −2.58 | 31.9 | 36.1 |
| I247K | −2.50 | 13.6 | 40.4 |
| I249H | −2.39 | 9.6 | 67.6 |
| I249S | −2.18 | 18.9 | 67.6 |
| I249Y | −2.10 | 10.5 | 76.2 |
| I249T | −2.04 | 3.6 | 67.6 |
| I249A | −1.95 | 6.5 | 67.6 |
| I249K | −1.85 | 0.4 | 67.6 |
| I249Q | −1.83 | 14.4 | 67.6 |
| I249F | −1.60 | 21.4 | 76.2 |
| I249R | −1.56 | 2.2 | 67.6 |
| I247V | −1.54 | 21.4 | 47.2 |
| I249V | −1.49 | 20.0 | 67.6 |
| I247Y | −1.34 | 22.0 | 52.2 |
| I249G | −1.29 | 5.2 | 67.6 |
| I249M | −1.11 | 24.9 | 77.9 |
| S246N | −0.39 | 17.3 | 75.0 |
| S246D | 0.16 | 0.5 | 65.9 |
| S246T | 0.17 | 35.3 | 64.3 |
| I249W | 0.22 | 3.1 | 76.2 |
| S246E | 0.39 | 0.0 | 74.2 |
| I247W | 0.63 | 26.6 | 39.1 |
| S246R | 0.82 | 35.0 | 64.0 |
| T243E | 0.96 | 15.2 | 68.5 |
| V242T | 1.19 | 6.1 | 49.1 |
| V242K | 1.80 | 15.5 | 53.1 |
| Y241H | 1.96 | 0.0 | 74.2 |
| S246G | 2.17 | 19.1 | 64.9 |
| V242E | 2.59 | 6.1 | 32.4 |
| S246P | 2.69 | 31.1 | 59.3 |
| I249P | 3.25 | 1.3 | 67.6 |
| Y241E | 3.38 | 0.0 | 27.4 |
| Y241D | 3.48 | 0.0 | 51.4 |
| V242A | 3.59 | 6.1 | 68.5 |
| V242Q | 3.73 | 15.5 | 72.1 |
| V242N | 3.73 | 15.2 | 74.1 |
| Y241A | 4.29 | 0.0 | 63.0 |
| Y241Q | 4.50 | 0.0 | 74.9 |
| V242D | 4.64 | 0.0 | 53.3 |

TABLE 5.B.v
Less immunogenic variants of RANKL agretope 15.

| | | Iscore | Iscore |
|---|---|---|---|
| Var. | E(PDA) | Anchor | Overlap |
| wt | 0.00 | 45.2 | 9.8 |
| F280Y | −1.69 | 29.3 | 9.8 |
| K282E | −0.21 | 0.0 | 9.4 |
| S285A | 0.16 | 27.1 | 9.4 |
| K282Q | 0.67 | 10.4 | 9.8 |
| S285E | 0.67 | 0.0 | 9.4 |
| S285D | 0.78 | 0.0 | 9.4 |
| K282A | 1.07 | 20.4 | 9.8 |
| K282D | 1.23 | 14.3 | 9.4 |
| L283E | 1.32 | 0.4 | 9.4 |
| S285Q | 1.34 | 20.6 | 9.4 |
| S285G | 1.40 | 13.6 | 9.4 |
| K282T | 1.50 | 38.8 | 9.8 |
| S285H | 1.58 | 13.1 | 9.4 |
| K282S | 1.72 | 37.0 | 9.8 |
| L283N | 1.73 | 12.2 | 9.4 |
| F280T | 2.09 | 9.5 | 9.4 |
| G279A | 2.11 | 39.8 | 9.8 |
| V277N | 2.18 | 0.0 | 9.8 |
| K282H | 2.36 | 18.7 | 9.8 |
| K282G | 2.44 | 29.5 | 9.8 |
| V277D | 2.84 | 0.0 | 9.8 |
| V277A | 3.11 | 0.0 | 9.8 |
| S285W | 3.24 | 1.1 | 9.4 |
| L283H | 3.46 | 14.2 | 9.4 |
| L283Q | 3.49 | 7.4 | 9.4 |
| L283D | 3.99 | 0.0 | 9.4 |
| V277P | 4.27 | 0.0 | 9.8 |
| L283T | 4.36 | 0.4 | 9.4 |
| L283S | 4.74 | 0.4 | 9.4 |
| L283A | 4.83 | 13.8 | 9.4 |

TABLE 5.B.vi
Less immunogenic variants of RANKL agretope 17.

| | | Iscore | Iscore |
|---|---|---|---|
| Var. | E(PDA) | Anchor | Overlap |
| wt | 0.00 | 18.5 | 0.0 |
| S297K | −0.99 | 8.6 | 0.0 |
| S297R | −0.72 | 3.4 | 0.0 |
| N295H | −0.57 | 8.6 | 0.0 |
| S297A | 0.13 | 8.6 | 0.0 |
| S294K | 0.14 | 0.0 | 0.0 |
| S294Q | 0.45 | 0.0 | 0.0 |
| S294R | 0.55 | 0.0 | 0.0 |
| S294E | 0.80 | 0.0 | 0.0 |
| S297D | 0.86 | 0.0 | 0.0 |
| N295E | 1.10 | 17.2 | 0.0 |
| I291V | 1.21 | 8.6 | 0.0 |
| S297G | 1.66 | 3.4 | 0.0 |
| S294I | 1.70 | 8.6 | 0.0 |
| S294A | 1.73 | 8.6 | 0.0 |
| E292A | 1.80 | 8.4 | 0.0 |
| E292S | 2.17 | 12.3 | 0.0 |
| I291P | 2.21 | 8.6 | 0.0 |
| N295Q | 2.30 | 8.6 | 0.0 |
| N295L | 2.46 | 17.2 | 0.0 |
| N295T | 2.49 | 9.9 | 0.0 |
| I291Q | 2.49 | 3.4 | 0.0 |
| N295K | 2.68 | 8.6 | 0.0 |
| I289T | 2.68 | 0.0 | 0.0 |
| I291T | 3.04 | 3.4 | 0.0 |
| N295R | 3.08 | 3.4 | 0.0 |
| N295D | 3.19 | 8.6 | 0.0 |
| I289K | 3.21 | 0.0 | 0.0 |
| I289N | 3.54 | 0.0 | 0.0 |
| I291E | 3.56 | 0.0 | 0.0 |
| N295A | 3.62 | 8.6 | 0.0 |
| S290D | 3.62 | 8.6 | 0.0 |
| I291K | 3.82 | 3.4 | 0.0 |
| I289A | 3.90 | 0.0 | 0.0 |
| E292G | 3.98 | 0.4 | 0.0 |
| S294H | 4.19 | 0.0 | 0.0 |
| N295S | 4.27 | 9.9 | 0.0 |
| I291N | 4.32 | 8.6 | 0.0 |
| I289S | 4.51 | 0.0 | 0.0 |
| I291A | 4.79 | 3.4 | 0.0 |

TABLE 5.C.i
Less immunogenic variants of APRIL agretope 5.

| | | Iscore | Iscore |
|---|---|---|---|
| Var. | E(PDA) | Anchor | Overlap |
| wt | 0.00 | 36.5 | 13.3 |
| L142A | 3.52 | 0.0 | 13.3 |
| L142D | 3.81 | 0.0 | 13.3 |
| L142E | 2.15 | 0.0 | 13.3 |
| L142F | 1.49 | 31.1 | 13.3 |
| L142G | 4.69 | 0.0 | 13.3 |
| L142H | 2.23 | 0.0 | 13.3 |
| L142K | 3.05 | 0.0 | 13.3 |
| L142N | 3.23 | 0.0 | 13.3 |
| L142Q | 3.35 | 0.0 | 13.3 |
| L142S | 4.28 | 0.0 | 13.3 |
| L142T | 3.11 | 0.0 | 13.3 |
| L142W | 4.37 | 31.1 | 13.3 |
| L142Y | 1.93 | 31.1 | 13.3 |
| R143A | −0.18 | 0.0 | 5.5 |
| R143D | 0.24 | 0.0 | 0.0 |
| R143E | −0.15 | 0.0 | 0.0 |
| R143F | 1.98 | 5.6 | 6.7 |
| R143G | 0.93 | 0.0 | 0.0 |
| R143H | 1.61 | 5.6 | 5.5 |
| R143M | 0.64 | 5.9 | 9.2 |
| R143N | −1.91 | 5.6 | 2.6 |
| R143P | −1.58 | 0.0 | 5.5 |
| R143Q | −1.30 | 14.3 | 2.2 |
| R143S | 0.13 | 0.0 | 5.5 |
| R143T | −0.40 | 0.0 | 11.9 |
| R143W | 2.95 | 0.0 | 6.7 |
| R143Y | 2.05 | 5.6 | 6.7 |
| R144A | −1.00 | 15.2 | 6.7 |
| R144D | −0.92 | 0.0 | 0.0 |
| R144E | −1.45 | 0.0 | 0.0 |
| R144G | −0.27 | 17.1 | 0.0 |
| R144H | −0.68 | 17.1 | 5.5 |
| R144K | 0.05 | 15.2 | 5.5 |
| R144N | −1.31 | 33.2 | 5.5 |
| R144P | 4.14 | 19.3 | 0.0 |
| R144Q | −1.22 | 15.2 | 3.7 |
| R144S | −1.37 | 17.1 | 8.8 |
| R144T | −1.33 | 15.2 | 1.2 |
| R144V | −1.83 | 33.2 | 8.1 |
| R144W | 1.63 | 15.2 | 5.5 |
| G147A | −1.63 | 30.1 | 13.3 |
| G147D | 4.67 | 14.3 | 13.3 |
| G147E | 1.41 | 5.6 | 13.3 |
| G147P | −2.40 | 33.9 | 13.3 |
| L148A | 4.23 | 5.9 | 13.3 |
| L148D | 2.78 | 0.0 | 13.3 |
| L148E | 2.54 | 1.2 | 13.3 |
| L148K | 1.52 | 3.1 | 13.3 |
| L148N | 2.46 | 10.6 | 13.3 |
| L148P | 1.37 | 13.9 | 13.3 |
| L148Q | 3.06 | 5.9 | 13.3 |
| L148S | 4.36 | 8.4 | 13.3 |
| A150G | 2.53 | 15.2 | 13.3 |
| A150K | 3.66 | 0.0 | 13.3 |
| A150P | 0.50 | 14.3 | 13.3 |
| A150S | 0.38 | 29.1 | 13.3 |
| A150T | 1.99 | 33.2 | 13.3 |

TABLE 5.C.ii
Less immunogenic variants of APRIL agretope 9.

| | | Iscore | Iscore |
|---|---|---|---|
| Var. | E(PDA) | Anchor | Overlap |
| wt | 0.00 | 35.4 | 49.5 |
| L164A | 3.67 | 0.0 | 32.1 |
| L164D | 4.25 | 0.0 | 26.2 |
| L164E | 2.77 | 0.0 | 26.2 |
| L164F | −0.19 | 35.3 | 48.1 |
| L164H | 2.10 | 0.0 | 34.7 |
| L164K | 0.56 | 0.0 | 39.2 |
| L164N | 4.13 | 0.0 | 40.3 |
| L164Q | 3.22 | 0.0 | 39.8 |
| L164S | 4.94 | 0.0 | 32.1 |
| L164Y | −2.17 | 35.3 | 48.1 |
| L165A | 2.37 | 13.6 | 46.5 |
| L165D | 2.49 | 0.0 | 42.0 |
| L165E | 2.63 | 13.6 | 30.7 |
| L165G | 4.83 | 18.8 | 33.2 |
| L165N | 1.00 | 25.5 | 37.4 |
| S167E | 3.86 | 23.7 | 29.9 |
| S167K | 0.45 | 34.1 | 33.6 |
| S167T | −0.44 | 28.7 | 48.0 |
| S167V | 3.73 | 30.5 | 47.5 |
| V169D | 5.00 | 0.0 | 45.7 |
| V169G | 3.53 | 0.0 | 49.3 |
| V169S | 1.90 | 26.0 | 46.2 |
| L170A | 2.01 | 15.6 | 25.0 |
| L170G | 0.53 | 8.6 | 4.9 |
| L170E | 3.01 | 8.6 | 4.9 |
| L170F | −1.24 | 15.3 | 27.0 |
| L170G | 4.88 | 5.2 | 7.1 |
| L170H | 0.76 | 11.9 | 17.8 |
| L170N | 2.18 | 26.0 | 6.3 |
| L170Q | 2.78 | 15.2 | 22.7 |
| L170S | 3.08 | 16.4 | 27.9 |
| L170T | 1.97 | 16.4 | 15.7 |
| L170Y | 0.80 | 7.6 | 22.9 |
| Q172A | −0.06 | 27.9 | 49.5 |
| Q172E | −1.41 | 5.2 | 36.4 |
| Q172G | 1.16 | 18.2 | 49.5 |
| Q172K | 0.14 | 21.0 | 49.5 |
| Q172N | 0.60 | 0.0 | 49.5 |
| Q172T | −0.49 | 10.7 | 49.5 |

TABLE 5.C.iii
Less immunogenic variants of APRIL agretope 10.

| | | Iscore | Iscore |
|---|---|---|---|
| Var. | E(PDA) | Anchor | Overlap |
| wt | 0.00 | 26.2 | 58.7 |
| L170A | 2.01 | 0.0 | 40.5 |
| L170D | 0.53 | 0.0 | 13.5 |
| L170E | 3.01 | 0.0 | 13.5 |
| L170F | −1.24 | 15.8 | 26.5 |
| L170G | 4.88 | 0.0 | 12.4 |
| L170H | 0.76 | 0.0 | 29.7 |
| L170N | 2.18 | 0.0 | 32.3 |
| L170Q | 2.78 | 0.0 | 37.9 |
| L170S | 3.08 | 0.0 | 44.4 |
| L170T | 1.97 | 0.0 | 32.1 |
| L170Y | 0.80 | 15.8 | 14.7 |
| Q172D | 0.61 | 13.1 | 49.5 |
| Q172E | −1.41 | 13.1 | 28.5 |
| D173E | 3.33 | 0.0 | 58.7 |
| D173N | 0.58 | 7.6 | 58.7 |
| D173T | 4.41 | 6.1 | 58.7 |
| T175D | 0.56 | 0.0 | 58.7 |
| T175E | 1.00 | 0.0 | 58.7 |
| T175G | 2.90 | 13.1 | 58.7 |
| T175S | 0.86 | 13.1 | 58.7 |
| T175Y | 3.15 | 0.0 | 58.7 |
| F176D | −2.14 | 15.8 | 58.7 |
| F176H | −0.33 | 15.8 | 58.7 |
| F176K | −0.51 | 15.8 | 58.7 |
| F176R | −0.15 | 13.1 | 58.7 |
| F176W | −0.13 | 13.1 | 58.7 |
| M178A | 4.85 | 13.1 | 58.7 |
| M178E | 4.13 | 13.1 | 58.7 |
| M178K | 2.64 | 13.1 | 58.7 |
| M178N | 4.80 | 5.2 | 58.7 |
| M178Q | 3.37 | 15.8 | 58.7 |
| M178T | 3.94 | 5.2 | 58.7 |

TABLE 5.C.iv
Less immunogenic variants of APRIL agretope 11.

| | | Iscore | Iscore |
|---|---|---|---|
| Var. | E(PDA) | Anchor | Overlap |
| wt | 0.00 | 56.7 | 7.5 |
| F194A | 2.04 | 0.0 | 7.5 |
| F194D | 3.83 | 0.0 | 7.5 |
| F194G | 4.42 | 0.0 | 7.5 |
| F194H | 4.32 | 0.0 | 7.5 |
| F194N | 3.40 | 0.0 | 7.5 |
| F194S | 2.28 | 0.0 | 7.5 |
| F194T | 2.74 | 0.0 | 7.5 |
| R195A | 0.39 | 17.8 | 7.5 |
| R195D | −0.16 | 0.3 | 7.5 |
| R195E | −1.22 | 17.8 | 7.5 |
| R195G | 2.17 | 27.9 | 7.5 |
| R195I | −0.93 | 36.6 | 7.5 |
| R195K | −0.73 | 36.6 | 7.5 |
| R195L | 3.22 | 36.1 | 7.5 |
| R195M | −1.47 | 36.6 | 7.5 |
| R195N | −0.35 | 35.8 | 7.5 |
| R195Q | −1.24 | 39.7 | 7.5 |
| R195S | 0.63 | 17.1 | 7.5 |
| R195T | −0.99 | 17.8 | 7.5 |
| R195V | −1.65 | 56.2 | 7.5 |
| I197A | 0.27 | 46.1 | 0.0 |
| I197D | −0.07 | 44.8 | 0.0 |
| I197E | −1.00 | 25.2 | 0.0 |
| I197G | 2.02 | 16.9 | 0.0 |
| I197H | 0.26 | 56.2 | 0.0 |
| I197K | 3.09 | 38.8 | 0.0 |
| I197N | 1.26 | 47.9 | 0.0 |
| I197Q | 0.98 | 49.0 | 0.0 |
| I197R | 3.80 | 33.2 | 0.0 |
| I197S | 1.05 | 52.8 | 0.0 |
| I197T | 0.11 | 45.0 | 0.0 |
| I197V | −0.65 | 49.6 | 7.5 |
| S199G | 3.35 | 42.9 | 7.5 |
| S199H | 1.50 | 34.5 | 7.5 |
| S199W | −0.80 | 26.4 | 7.5 |
| M200A | −3.91 | 45.5 | 0.4 |
| M200D | −5.12 | 22.4 | 0.0 |
| M200E | −3.68 | 31.2 | 3.4 |
| M200G | 0.13 | 19.4 | 0.0 |
| M200K | −5.10 | 39.9 | 3.1 |
| M200N | −8.27 | 43.1 | 0.0 |
| M200Q | −6.10 | 38.6 | 0.4 |
| M200S | −5.91 | 34.8 | 0.0 |
| M200T | −3.41 | 35.2 | 0.0 |
| S202D | −0.39 | 20.8 | 0.4 |
| S202E | −0.76 | 24.7 | 0.0 |
| S202G | −0.11 | 38.5 | 0.0 |
| S202H | 1.29 | 53.4 | 1.3 |
| S202N | 0.17 | 26.1 | 7.0 |
| S202Q | −0.01 | 42.3 | 1.3 |
| S202W | 2.60 | 40.1 | 5.7 |
| S202Y | 1.69 | 56.6 | 5.2 |

TABLE 5.D.i
Less immunogenic variants of CD40L agretope 12.

| | | Iscore | Iscore |
|---|---|---|---|
| Var. | E(PDA) | Anchor | Overlap |
| wt | 0.00 | 48.3 | 16.9 |
| L206A | 4.78 | 0.0 | 12.3 |
| L206D | 4.72 | 0.0 | 0.0 |
| L206E | 0.74 | 0.0 | 4.3 |
| L206N | 3.82 | 0.0 | 15.4 |
| L206T | 4.91 | 0.0 | 12.5 |
| R207A | 3.31 | 2.5 | 7.5 |
| R207D | 2.19 | 0.0 | 13.1 |
| R207E | 2.96 | 2.5 | 10.0 |
| R207K | 1.61 | 16.7 | 9.8 |
| R207N | 1.51 | 9.4 | 15.8 |
| R207Q | 1.46 | 17.4 | 10.7 |
| R207S | 1.93 | 0.4 | 8.7 |
| R207T | 3.41 | 2.5 | 8.7 |
| A209G | 0.86 | 10.2 | 12.4 |
| T211D | −1.51 | 0.0 | 9.2 |
| T211E | −1.01 | 0.0 | 16.8 |
| T211F | 1.17 | 0.0 | 11.2 |
| T211G | 2.35 | 3.5 | 6.0 |
| T211K | 2.72 | 37.8 | 10.1 |
| T211R | 2.80 | 30.7 | 13.7 |
| T211Y | −0.55 | 0.0 | 10.8 |
| H212D | 4.55 | 16.7 | 13.6 |
| H212F | 1.50 | 46.2 | 15.3 |
| H212N | 3.76 | 46.9 | 12.4 |
| S214A | 1.01 | 18.1 | 16.9 |
| S214D | 0.28 | 1.5 | 16.9 |
| S214E | 0.21 | 1.5 | 16.9 |
| S214F | 4.42 | 13.7 | 16.9 |
| S214G | 1.84 | 11.1 | 16.9 |
| S214H | 1.19 | 16.7 | 16.9 |
| S214I | 2.11 | 29.0 | 16.9 |
| S214K | 1.70 | 15.8 | 16.9 |
| S214L | 3.12 | 11.1 | 16.9 |
| S214M | 2.87 | 30.5 | 16.9 |
| S214N | 0.83 | 3.6 | 16.9 |
| S214P | −0.23 | 2.5 | 16.9 |
| S214Q | 1.20 | 32.3 | 16.9 |
| S214R | 1.76 | 10.8 | 16.9 |
| S214T | 1.22 | 2.5 | 16.9 |
| S214V | 1.20 | 14.8 | 16.9 |
| S214W | 3.73 | 2.9 | 16.9 |
| S214Y | 3.88 | 9.4 | 16.9 |

TABLE 5.E.i
Less immunogenic variants of TRAIL agretope 2.

| | | Iscore | Iscore |
|---|---|---|---|
| Var. | E(PDA) | Anchor | Overlap |
| wt | 0.00 | 46.0 | 22.2 |
| L174E | 3.54 | 0.0 | 22.2 |
| L174Q | 4.46 | 0.0 | 22.2 |
| V175A | 2.45 | 0.0 | 22.2 |
| V175D | 2.36 | 0.0 | 22.2 |
| V175E | 1.99 | 2.6 | 22.2 |
| V175G | 4.63 | 11.4 | 22.2 |
| V175I | 1.79 | 22.7 | 22.2 |
| V175K | 2.19 | 22.7 | 22.2 |
| V175N | 2.48 | 20.0 | 22.2 |
| V175Q | 1.86 | 29.1 | 22.2 |
| V175S | 2.08 | 0.0 | 22.2 |
| V175T | 1.85 | 0.0 | 22.2 |
| I176L | 4.00 | 40.8 | 22.2 |
| I176N | 4.62 | 22.7 | 22.2 |
| I176T | 3.41 | 12.9 | 22.2 |
| I176V | 0.65 | 22.7 | 22.2 |
| H177D | −0.64 | 26.2 | 22.2 |
| H177E | −1.84 | 13.1 | 22.2 |
| H177N | −1.84 | 41.2 | 22.2 |
| H177T | −0.48 | 13.9 | 22.2 |
| H177W | 1.19 | 34.4 | 22.2 |
| K179A | 2.42 | 23.5 | 22.2 |
| K179D | 1.13 | 0.0 | 22.2 |
| K179E | −0.20 | 0.0 | 22.2 |
| K179G | 0.98 | 16.2 | 22.2 |
| K179H | 1.99 | 20.0 | 22.2 |
| K179N | 1.04 | 25.8 | 22.2 |
| K179P | −1.03 | 29.1 | 22.2 |
| K179Q | −0.01 | 11.4 | 22.2 |
| K179S | −0.58 | 31.4 | 22.2 |

TABLE 5.E.ii
Less immunogenic variants of TRAIL agretope 7.

| | | Iscore | Iscore |
|---|---|---|---|
| Var. | E(PDA) | Anchor | Overlap |
| wt | 0.00 | 47.2 | 15.5 |
| V207A | 3.18 | 0.0 | 1.3 |
| V207D | 2.82 | 0.0 | 1.3 |
| V207E | 3.43 | 0.0 | 1.3 |
| V207K | 3.60 | 0.0 | 1.3 |
| V207N | 1.45 | 0.0 | 1.3 |
| V207Q | 2.31 | 0.0 | 1.3 |
| V207S | 3.86 | 0.0 | 1.3 |
| V207T | 1.86 | 0.0 | 1.3 |
| Q208A | 1.05 | 9.9 | 15.5 |
| Q208D | −0.04 | 0.0 | 1.3 |
| Q208E | −0.91 | 9.9 | 1.3 |
| Q208T | 3.88 | 9.9 | 15.5 |
| Y209E | 4.60 | 1.1 | 0.0 |
| Y209H | 3.29 | 20.8 | 0.0 |
| Y209K | 4.53 | 20.8 | 0.0 |
| I210E | 4.71 | 2.9 | 14.3 |
| I210K | 4.80 | 33.5 | 15.5 |
| I210L | 3.04 | 44.3 | 15.5 |
| I210N | 4.45 | 36.0 | 14.3 |
| I210Q | 2.45 | 38.3 | 15.5 |
| I210T | 4.22 | 7.9 | 14.3 |
| K212G | 4.84 | 12.3 | 14.8 |
| Y213H | 3.26 | 38.9 | 15.5 |
| Y213K | 4.85 | 36.2 | 15.5 |
| S215D | −0.24 | 0.0 | 14.3 |
| S215E | −0.27 | 6.2 | 14.3 |
| S215T | 0.50 | 1.1 | 15.5 |

TABLE 5.E.iii
Less immunogenic variants of TRAIL agretope 10.

| | | Iscore | Iscore |
|---|---|---|---|
| Var. | E(PDA) | Anchor | Overlap |
| wt | 0.00 | 28.5 | 9.1 |
| L221A | 4.71 | 0.0 | 4.0 |
| L221K | 4.03 | 0.0 | 9.1 |
| L221N | 3.25 | 0.0 | 8.6 |
| L221P | 2.86 | 0.0 | 1.9 |
| L221T | 4.75 | 0.0 | 4.0 |
| L222E | 2.83 | 22.3 | 1.4 |
| L222T | 4.97 | 22.3 | 4.0 |
| M223Q | 4.87 | 14.1 | 0.0 |
| K224A | 0.97 | 27.7 | 7.6 |
| K224E | 1.61 | 13.2 | 7.6 |
| K224G | 3.12 | 6.7 | 7.6 |
| K224S | 1.60 | 15.0 | 7.6 |
| K224T | 1.21 | 9.3 | 7.6 |
| A226G | 4.02 | 26.2 | 1.9 |
| R227A | 0.51 | 26.4 | 9.1 |
| R227D | −0.27 | 0.4 | 9.1 |
| R227E | −2.18 | 3.6 | 9.1 |
| R227F | 4.22 | 27.7 | 9.1 |
| R227G | 1.50 | 3.5 | 9.1 |
| R227K | −1.11 | 22.9 | 9.1 |
| R227Q | −2.75 | 13.8 | 9.1 |
| R227Y | 2.44 | 17.6 | 9.1 |
| S229A | 0.48 | 22.3 | 7.6 |
| S229G | 1.10 | 26.8 | 7.6 |
| S229T | 1.84 | 11.2 | 7.6 |

TABLE 5.E.iv
Less immunogenic variants of TRAIL agretope 14.

| | | Iscore | Iscore |
|---|---|---|---|
| Var. | E(PDA) | Anchor | Overlap |
| wt | 0.00 | 21.8 | 33.9 |
| I256A | 4.28 | 0.0 | 33.9 |
| I256K | 4.54 | 0.0 | 33.9 |
| I256N | 4.88 | 0.0 | 33.9 |
| I256Q | 4.15 | 0.0 | 33.9 |
| I256T | 2.70 | 0.0 | 33.9 |
| V258A | 4.82 | 10.2 | 0.0 |
| V258P | 0.48 | 13.2 | 0.0 |
| V258S | 4.73 | 13.2 | 0.0 |
| V258T | 1.49 | 10.2 | 0.0 |
| S259A | −1.64 | 18.7 | 33.6 |
| S259G | 2.50 | 1.0 | 33.9 |
| T261E | 3.90 | 0.0 | 33.9 |
| T261N | 4.15 | 10.2 | 33.9 |
| T261Q | 4.76 | 0.0 | 33.9 |
| T261S | 3.39 | 9.9 | 33.9 |
| N262D | 0.02 | 8.6 | 6.1 |
| N262E | −0.35 | 8.6 | 6.1 |
| N262F | 0.02 | 2.3 | 30.3 |
| N262H | −1.07 | 10.2 | 30.3 |
| N262K | 1.84 | 8.6 | 15.2 |
| N262Q | 2.18 | 10.7 | 15.2 |
| N262R | 1.59 | 2.5 | 15.5 |
| N262W | 3.07 | 1.0 | 30.3 |
| N262Y | 0.54 | 2.5 | 15.2 |
| H264A | 0.99 | 20.9 | 33.9 |
| H264D | 0.63 | 1.1 | 33.9 |
| H264E | −0.12 | 1.1 | 33.9 |
| H264G | 1.13 | 4.7 | 33.9 |
| H264K | 1.71 | 12.3 | 33.9 |
| H264L | 2.22 | 18.7 | 33.9 |
| H264M | 2.04 | 20.4 | 33.9 |
| H264N | 0.56 | 3.8 | 33.9 |
| H264P | 2.79 | 3.4 | 33.9 |
| H264R | 1.98 | 4.7 | 33.9 |
| H264T | 1.36 | 3.4 | 33.9 |
| H264V | 1.76 | 14.8 | 33.9 |

TABLE 5.E.v
Less immunogenic variants of TRAIL agretope 15.

| | | Iscore | Iscore |
|---|---|---|---|
| Var. | E(PDA) | Anchor | Overlap |
| wt | 0.00 | 33.9 | 21.8 |
| F257H | 3.50 | 0.0 | 21.8 |
| V258A | 4.82 | 0.0 | 10.2 |
| V258N | 4.72 | 15.2 | 21.8 |
| V258P | 0.48 | 0.0 | 13.2 |
| V258S | 4.73 | 0.0 | 13.2 |
| V258T | 1.49 | 0.0 | 10.2 |
| S259A | −1.64 | 33.6 | 18.7 |
| V260A | 4.64 | 24.0 | 21.8 |
| V260D | 2.13 | 17.2 | 21.8 |
| V260N | 4.81 | 24.4 | 21.8 |
| N262D | 0.02 | 6.1 | 8.6 |
| N262E | −0.35 | 6.1 | 8.6 |
| N262F | 0.02 | 30.3 | 2.3 |
| N262H | −1.07 | 30.3 | 10.2 |
| N262K | 1.84 | 15.2 | 8.6 |
| N262Q | 2.18 | 15.2 | 10.7 |
| N262R | 1.59 | 15.5 | 2.5 |
| N262W | 3.07 | 30.3 | 1.0 |
| N262Y | 0.54 | 15.2 | 2.5 |
| E263G | 2.63 | 16.6 | 21.8 |
| E263K | 0.22 | 21.7 | 21.8 |
| E263P | −1.97 | 22.1 | 21.8 |
| L265A | 3.27 | 12.1 | 21.8 |
| L265D | 2.41 | 2.2 | 21.8 |
| L265E | 0.97 | 2.2 | 21.8 |
| L265F | 3.23 | 16.5 | 21.8 |
| L265G | 5.00 | 1.8 | 21.8 |
| L265H | 2.95 | 13.2 | 21.8 |
| L265K | 1.90 | 5.4 | 21.8 |
| L265M | 4.40 | 20.2 | 21.8 |
| L265N | 2.16 | 1.4 | 21.8 |
| L265Q | 2.61 | 15.0 | 21.8 |
| L265R | 4.55 | 0.7 | 21.8 |
| L265S | 3.18 | 23.4 | 21.8 |
| L265Y | 2.71 | 1.8 | 21.8 |

### Example 4

### MHC AgretoDes in TNF SF Member Variants Designed for Soluble Expression, Superagonism, Dominant Negative Inhibition, and Competitive Inhibition

Previously described TNF SF member variants have been designed for a number of improved properties, including but not limited to superagonism, dominant negative inhibition, competitive inhibition, and receptor specificity. All 9-mers for which Iscore is altered, relative to wild type, in one or more variants is shown below.

TABLE 10
Iscores of MHC agretopes in wild type human BAFF versus
designed BAFF variants.

| Var. | 170-178 | 171-179 | 203-211 | 207-215 | 208-216 | 213-221 | 216-224 | 217-225 | 228-236 | 234-242 |
|---|---|---|---|---|---|---|---|---|---|---|
| WT | 0 | 2.8 | 0 | 0 | 26 | 0 | 0 | 0 | 0 | 28 |
| Q167E | 0 | 2.8 | 0 | 0 | 26 | 0 | 0 | 0 | 0 | 28 |
| Q167K | 0 | 2.8 | 0 | 0 | 26 | 0 | 0 | 0 | 0 | 28 |
| Q167R | 0 | 2.8 | 0 | 0 | 26 | 0 | 0 | 0 | 0 | 28 |
| Q167D | 0 | 2.8 | 0 | 0 | 26 | 0 | 0 | 0 | 0 | 28 |
| S170N | 0 | 2.8 | 0 | 0 | 26 | 0 | 0 | 0 | 0 | 28 |
| S170L | 5.6 | 2.8 | 0 | 0 | 26 | 0 | 0 | 0 | 0 | 28 |
| S170D | 0 | 2.8 | 0 | 0 | 26 | 0 | 0 | 0 | 0 | 28 |
| Y171A | 0 | 0 | 0 | 0 | 26 | 0 | 0 | 0 | 0 | 28 |
| Y171E | 0 | 0 | 0 | 0 | 26 | 0 | 0 | 0 | 0 | 28 |
| Y171H | 0 | 0 | 0 | 0 | 26 | 0 | 0 | 0 | 0 | 28 |
| Y171K | 0 | 0 | 0 | 0 | 26 | 0 | 0 | 0 | 0 | 28 |
| Y171R | 0 | 0 | 0 | 0 | 26 | 0 | 0 | 0 | 0 | 28 |
| Y171D | 0 | 0 | 0 | 0 | 26 | 0 | 0 | 0 | 0 | 28 |
| Y171T | 0 | 0 | 0 | 0 | 26 | 0 | 0 | 0 | 0 | 28 |
| Y171F | 0 | 2.8 | 0 | 0 | 26 | 0 | 0 | 0 | 0 | 28 |
| Y171L | 0 | 0 | 0 | 0 | 26 | 0 | 0 | 0 | 0 | 28 |
| Y171I | 0 | 0 | 0 | 0 | 26 | 0 | 0 | 0 | 0 | 28 |
| D211S | 0 | 2.8 | 0.4 | 0 | 10 | 0 | 0 | 0 | 0 | 28 |
| D211N | 0 | 2.8 | 0 | 0 | 0.4 | 0 | 0 | 0 | 0 | 28 |
| D211E | 0 | 2.8 | 0 | 0 | 0 | 0 | 0 | 0 | 0 | 28 |
| D211K | 0 | 2.8 | 0 | 0 | 3.6 | 0 | 0 | 0 | 0 | 28 |
| D211G | 0 | 2.8 | 0 | 0 | 0.4 | 0 | 0 | 0 | 0 | 28 |
| K212E | 0 | 2.8 | 0 | 0 | 26 | 0 | 0 | 0 | 0 | 28 |
| K212Q | 0 | 2.8 | 0 | 0 | 26 | 0 | 0 | 0 | 0 | 28 |
| T213A | 0 | 2.8 | 0 | 1.1 | 13 | 0 | 0 | 0 | 0 | 28 |
| T213K | 0 | 2.8 | 0 | 0 | 26 | 0 | 0 | 0 | 0 | 28 |
| T213N | 0 | 2.8 | 0 | 0 | 5.2 | 0 | 0 | 0 | 0 | 28 |
| T213S | 0 | 2.8 | 0 | 0 | 13 | 0 | 0 | 0 | 0 | 28 |
| T213D | 0 | 2.8 | 0 | 0 | 0 | 0 | 0 | 0 | 0 | 28 |
| T213I | 0 | 2.8 | 0 | 5.4 | 26 | 11 | 0 | 0 | 0 | 28 |
| T213L | 0 | 2.8 | 0 | 13 | 13 | 11 | 0 | 0 | 0 | 28 |
| Y214A | 0 | 2.8 | 0 | 0 | 33 | 0 | 0 | 0 | 0 | 28 |
| Y214E | 0 | 2.8 | 0 | 0 | 33 | 0 | 0 | 0 | 0 | 28 |
| Y214K | 0 | 2.8 | 0 | 0 | 16 | 0 | 0 | 0 | 0 | 28 |
| Y214Q | 0 | 2.8 | 0 | 0 | 33 | 0 | 0 | 0 | 0 | 28 |
| Y214S | 0 | 2.8 | 0 | 0 | 29 | 0 | 0 | 0 | 0 | 28 |
| Y214F | 0 | 2.8 | 0 | 0 | 26 | 0 | 0 | 0 | 0 | 28 |
| Y214I | 0 | 2.8 | 0 | 0 | 33 | 0 | 0 | 0 | 0 | 28 |
| A215S | 0 | 2.8 | 0 | 0 | 26 | 0 | 0 | 0 | 0 | 28 |
| A215T | 0 | 2.8 | 0 | 0 | 26 | 0 | 0 | 0 | 0 | 28 |
| G217A | 0 | 2.8 | 0 | 0 | 26 | 0 | 0 | 0 | 0 | 28 |
| G217V | 0 | 2.8 | 0 | 0 | 26 | 0 | 3.6 | 1.1 | 0 | 28 |
| G217S | 0 | 2.8 | 0 | 0 | 26 | 0 | 0 | 0 | 0 | 28 |
| G217T | 0 | 2.8 | 0 | 0 | 26 | 0 | 0 | 0 | 0 | 28 |
| L219K | 0 | 2.8 | 0 | 0 | 26 | 0 | 0 | 0 | 0 | 28 |
| L219V | 0 | 2.8 | 0 | 0 | 26 | 0 | 0 | 0 | 0 | 28 |
| L219D | 0 | 2.8 | 0 | 0 | 26 | 0 | 0 | 0 | 0 | 28 |
| L219E | 0 | 2.8 | 0 | 0 | 26 | 0 | 0 | 0 | 0 | 28 |
| T236N | 0 | 2.8 | 0 | 0 | 26 | 0 | 0 | 0 | 0 | 36 |
| T236V | 0 | 2.8 | 0 | 0 | 26 | 0 | 0 | 0 | 0 | 36 |
| T236K | 0 | 2.8 | 0 | 0 | 26 | 0 | 0 | 0 | 0 | 28 |
| T236Q | 0 | 2.8 | 0 | 0 | 26 | 0 | 0 | 0 | 8.6 | 28 |
| T236E | 0 | 2.8 | 0 | 0 | 26 | 0 | 0 | 0 | 0 | 11 |
| T236D | 0 | 2.8 | 0 | 0 | 26 | 0 | 0 | 0 | 0 | 6.8 |
| R239A | 0 | 2.8 | 0 | 0 | 26 | 0 | 0 | 0 | 0 | 20 |
| R239K | 0 | 2.8 | 0 | 0 | 26 | 0 | 0 | 0 | 0 | 31 |
| R239E | 0 | 2.8 | 0 | 0 | 26 | 0 | 0 | 0 | 0 | 0 |
| R239D | 0 | 2.8 | 0 | 0 | 26 | 0 | 0 | 0 | 0 | 1.5 |
| I241A | 0 | 2.8 | 0 | 0 | 26 | 0 | 0 | 0 | 0 | 28 |
| I241E | 0 | 2.8 | 0 | 0 | 26 | 0 | 0 | 0 | 0 | 28 |
| I241L | 0 | 2.8 | 0 | 0 | 26 | 0 | 0 | 0 | 0 | 28 |
| I241T | 0 | 2.8 | 0 | 0 | 26 | 0 | 0 | 0 | 0 | 28 |
| I241V | 0 | 2.8 | 0 | 0 | 26 | 0 | 0 | 0 | 0 | 28 |
| I241Q | 0 | 2.8 | 0 | 0 | 26 | 0 | 0 | 0 | 0 | 28 |
| I241Y | 0 | 2.8 | 0 | 0 | 26 | 0 | 0 | 0 | 0 | 28 |
| E246Q | 0 | 2.8 | 0 | 0 | 26 | 0 | 0 | 0 | 0 | 28 |
| E246K | 0 | 2.8 | 0 | 0 | 26 | 0 | 0 | 0 | 0 | 28 |
| L248A | 0 | 2.8 | 0 | 0 | 26 | 0 | 0 | 0 | 0 | 28 |
| L248E | 0 | 2.8 | 0 | 0 | 26 | 0 | 0 | 0 | 0 | 28 |
| L248K | 0 | 2.8 | 0 | 0 | 26 | 0 | 0 | 0 | 0 | 28 |
| L248N | 0 | 2.8 | 0 | 0 | 26 | 0 | 0 | 0 | 0 | 28 |
| L248Q | 0 | 2.8 | 0 | 0 | 26 | 0 | 0 | 0 | 0 | 28 |
| L248R | 0 | 2.8 | 0 | 0 | 26 | 0 | 0 | 0 | 0 | 28 |
| L248S | 0 | 2.8 | 0 | 0 | 26 | 0 | 0 | 0 | 0 | 28 |
| L248Y | 0 | 2.8 | 0 | 0 | 26 | 0 | 0 | 0 | 0 | 28 |
| L248F | 0 | 2.8 | 0 | 0 | 26 | 0 | 0 | 0 | 0 | 28 |
| N250A | 0 | 2.8 | 0 | 0 | 26 | 0 | 0 | 0 | 0 | 28 |
| N250S | 0 | 2.8 | 0 | 0 | 26 | 0 | 0 | 0 | 0 | 28 |
| N250D | 0 | 2.8 | 0 | 0 | 26 | 0 | 0 | 0 | 0 | 28 |
| N250Y | 0 | 2.8 | 0 | 0 | 26 | 0 | 0 | 0 | 0 | 28 |
| P272N | 0 | 2.8 | 0 | 0 | 26 | 0 | 0 | 0 | 0 | 28 |
| P272D | 0 | 2.8 | 0 | 0 | 26 | 0 | 0 | 0 | 0 | 28 |
| P272S | 0 | 2.8 | 0 | 0 | 26 | 0 | 0 | 0 | 0 | 28 |
| P272A | 0 | 2.8 | 0 | 0 | 26 | 0 | 0 | 0 | 0 | 28 |
| R273A | 0 | 2.8 | 0 | 0 | 26 | 0 | 0 | 0 | 0 | 28 |
| R273K | 0 | 2.8 | 0 | 0 | 26 | 0 | 0 | 0 | 0 | 28 |
| R273L | 0 | 2.8 | 0 | 0 | 26 | 0 | 0 | 0 | 0 | 28 |
| R273E | 0 | 2.8 | 0 | 0 | 26 | 0 | 0 | 0 | 0 | 28 |
| R273H | 0 | 2.8 | 0 | 0 | 26 | 0 | 0 | 0 | 0 | 28 |
| E274A | 0 | 2.8 | 0 | 0 | 26 | 0 | 0 | 0 | 0 | 28 |
| E274L | 0 | 2.8 | 0 | 0 | 26 | 0 | 0 | 0 | 0 | 28 |
| E274Q | 0 | 2.8 | 0 | 0 | 26 | 0 | 0 | 0 | 0 | 28 |
| E274T | 0 | 2.8 | 0 | 0 | 26 | 0 | 0 | 0 | 0 | 28 |
| E274K | 0 | 2.8 | 0 | 0 | 26 | 0 | 0 | 0 | 0 | 28 |
| E274R | 0 | 2.8 | 0 | 0 | 26 | 0 | 0 | 0 | 0 | 28 |
| E274D | 0 | 2.8 | 0 | 0 | 26 | 0 | 0 | 0 | 0 | 28 |
| E274I | 0 | 2.8 | 0 | 0 | 26 | 0 | 0 | 0 | 0 | 28 |
| E274F | 0 | 2.8 | 0 | 0 | 26 | 0 | 0 | 0 | 0 | 28 |
| N275D | 0 | 2.8 | 0 | 0 | 26 | 0 | 0 | 0 | 0 | 28 |
| N275E | 0 | 2.8 | 0 | 0 | 26 | 0 | 0 | 0 | 0 | 28 |
| N275R | 0 | 2.8 | 0 | 0 | 26 | 0 | 0 | 0 | 0 | 28 |
| N275S | 0 | 2.8 | 0 | 0 | 26 | 0 | 0 | 0 | 0 | 28 |
| Q277H | 0 | 2.8 | 0 | 0 | 26 | 0 | 0 | 0 | 0 | 28 |
| Q277K | 0 | 2.8 | 0 | 0 | 26 | 0 | 0 | 0 | 0 | 28 |
| Q277S | 0 | 2.8 | 0 | 0 | 26 | 0 | 0 | 0 | 0 | 28 |
| Q277R | 0 | 2.8 | 0 | 0 | 26 | 0 | 0 | 0 | 0 | 28 |
| Q277E | 0 | 2.8 | 0 | 0 | 26 | 0 | 0 | 0 | 0 | 28 |
| S279R | 0 | 2.8 | 0 | 0 | 26 | 0 | 0 | 0 | 0 | 28 |
| S279E | 0 | 2.8 | 0 | 0 | 26 | 0 | 0 | 0 | 0 | 28 |
| D281A | 0 | 2.8 | 0 | 0 | 26 | 0 | 0 | 0 | 0 | 28 |
| D281E | 0 | 2.8 | 0 | 0 | 26 | 0 | 0 | 0 | 0 | 28 |
| D281S | 0 | 2.8 | 0 | 0 | 26 | 0 | 0 | 0 | 0 | 28 |
| D281K | 0 | 2.8 | 0 | 0 | 26 | 0 | 0 | 0 | 0 | 28 |
| D281R | 0 | 2.8 | 0 | 0 | 26 | 0 | 0 | 0 | 0 | 28 |
| D281H | 0 | 2.8 | 0 | 0 | 26 | 0 | 0 | 0 | 0 | 28 |
| D281N | 0 | 2.8 | 0 | 0 | 26 | 0 | 0 | 0 | 0 | 28 |
| D283A | 0 | 2.8 | 0 | 0 | 26 | 0 | 0 | 0 | 0 | 28 |
| D283V | 0 | 2.8 | 0 | 0 | 26 | 0 | 0 | 0 | 0 | 28 |
| D283K | 0 | 2.8 | 0 | 0 | 26 | 0 | 0 | 0 | 0 | 28 |
| D283R | 0 | 2.8 | 0 | 0 | 26 | 0 | 0 | 0 | 0 | 28 |
| D283H | 0 | 2.8 | 0 | 0 | 26 | 0 | 0 | 0 | 0 | 28 |
| D283N | 0 | 2.8 | 0 | 0 | 26 | 0 | 0 | 0 | 0 | 28 |
| D283E | 0 | 2.8 | 0 | 0 | 26 | 0 | 0 | 0 | 0 | 28 |
| Var. | 235-243 | 236-244 | 238-246 | 250-258 | 267-275 | 269-277 | 271-279 | 274-282 | 278-286 | 280-288 |
| WT | 5.6 | 0 | 17 | 0 | 3.3 | 0 | 0 | 0 | 0 | 0 |
| Q167E | 5.6 | 0 | 17 | 0 | 3.3 | 0 | 0 | 0 | 0 | 0 |
| Q167K | 5.6 | 0 | 17 | 0 | 3.3 | 0 | 0 | 0 | 0 | 0 |
| Q167R | 5.6 | 0 | 17 | 0 | 3.3 | 0 | 0 | 0 | 0 | 0 |
| Q167D | 5.6 | 0 | 17 | 0 | 3.3 | 0 | 0 | 0 | 0 | 0 |
| S170N | 5.6 | 0 | 17 | 0 | 3.3 | 0 | 0 | 0 | 0 | 0 |
| S170L | 5.6 | 0 | 17 | 0 | 3.3 | 0 | 0 | 0 | 0 | 0 |
| S170D | 5.6 | 0 | 17 | 0 | 3.3 | 0 | 0 | 0 | 0 | 0 |
| Y171A | 5.6 | 0 | 17 | 0 | 3.3 | 0 | 0 | 0 | 0 | 0 |
| Y171E | 5.6 | 0 | 17 | 0 | 3.3 | 0 | 0 | 0 | 0 | 0 |
| Y171H | 5.6 | 0 | 17 | 0 | 3.3 | 0 | 0 | 0 | 0 | 0 |
| Y171K | 5.6 | 0 | 17 | 0 | 3.3 | 0 | 0 | 0 | 0 | 0 |
| Y171R | 5.6 | 0 | 17 | 0 | 3.3 | 0 | 0 | 0 | 0 | 0 |
| Y171D | 5.6 | 0 | 17 | 0 | 3.3 | 0 | 0 | 0 | 0 | 0 |
| Y171T | 5.6 | 0 | 17 | 0 | 3.3 | 0 | 0 | 0 | 0 | 0 |
| Y171F | 5.6 | 0 | 17 | 0 | 3.3 | 0 | 0 | 0 | 0 | 0 |
| Y171L | 5.6 | 0 | 17 | 0 | 3.3 | 0 | 0 | 0 | 0 | 0 |
| Y171I | 5.6 | 0 | 17 | 0 | 3.3 | 0 | 0 | 0 | 0 | 0 |
| D211S | 5.6 | 0 | 17 | 0 | 3.3 | 0 | 0 | 0 | 0 | 0 |
| D211N | 5.6 | 0 | 17 | 0 | 3.3 | 0 | 0 | 0 | 0 | 0 |
| D211E | 5.6 | 0 | 17 | 0 | 3.3 | 0 | 0 | 0 | 0 | 0 |
| D211K | 5.6 | 0 | 17 | 0 | 3.3 | 0 | 0 | 0 | 0 | 0 |
| D211G | 5.6 | 0 | 17 | 0 | 3.3 | 0 | 0 | 0 | 0 | 0 |
| K212E | 5.6 | 0 | 17 | 0 | 3.3 | 0 | 0 | 0 | 0 | 0 |
| K212Q | 5.6 | 0 | 17 | 0 | 3.3 | 0 | 0 | 0 | 0 | 0 |
| T213A | 5.6 | 0 | 17 | 0 | 3.3 | 0 | 0 | 0 | 0 | 0 |
| T213K | 5.6 | 0 | 17 | 0 | 3.3 | 0 | 0 | 0 | 0 | 0 |
| T213N | 5.6 | 0 | 17 | 0 | 3.3 | 0 | 0 | 0 | 0 | 0 |
| T213S | 5.6 | 0 | 17 | 0 | 3.3 | 0 | 0 | 0 | 0 | 0 |
| T213D | 5.6 | 0 | 17 | 0 | 3.3 | 0 | 0 | 0 | 0 | 0 |
| T213I | 5.6 | 0 | 17 | 0 | 3.3 | 0 | 0 | 0 | 0 | 0 |
| T213L | 5.6 | 0 | 17 | 0 | 3.3 | 0 | 0 | 0 | 0 | 0 |
| Y214A | 5.6 | 0 | 17 | 0 | 3.3 | 0 | 0 | 0 | 0 | 0 |
| Y214E | 5.6 | 0 | 17 | 0 | 3.3 | 0 | 0 | 0 | 0 | 0 |
| Y214K | 5.6 | 0 | 17 | 0 | 3.3 | 0 | 0 | 0 | 0 | 0 |
| Y214Q | 5.6 | 0 | 17 | 0 | 3.3 | 0 | 0 | 0 | 0 | 0 |
| Y214S | 5.6 | 0 | 17 | 0 | 3.3 | 0 | 0 | 0 | 0 | 0 |
| Y214F | 5.6 | 0 | 17 | 0 | 3.3 | 0 | 0 | 0 | 0 | 0 |
| Y214I | 5.6 | 0 | 17 | 0 | 3.3 | 0 | 0 | 0 | 0 | 0 |
| A215S | 5.6 | 0 | 17 | 0 | 3.3 | 0 | 0 | 0 | 0 | 0 |
| A215T | 5.6 | 0 | 17 | 0 | 3.3 | 0 | 0 | 0 | 0 | 0 |
| G217A | 5.6 | 0 | 17 | 0 | 3.3 | 0 | 0 | 0 | 0 | 0 |
| G217V | 5.6 | 0 | 17 | 0 | 3.3 | 0 | 0 | 0 | 0 | 0 |
| G217S | 5.6 | 0 | 17 | 0 | 3.3 | 0 | 0 | 0 | 0 | 0 |
| G217T | 5.6 | 0 | 17 | 0 | 3.3 | 0 | 0 | 0 | 0 | 0 |
| L219K | 5.6 | 0 | 17 | 0 | 3.3 | 0 | 0 | 0 | 0 | 0 |
| L219V | 5.6 | 0 | 17 | 0 | 3.3 | 0 | 0 | 0 | 0 | 0 |
| L219D | 5.6 | 0 | 17 | 0 | 3.3 | 0 | 0 | 0 | 0 | 0 |
| L219E | 5.6 | 0 | 17 | 0 | 3.3 | 0 | 0 | 0 | 0 | 0 |
| T236N | 14 | 0 | 17 | 0 | 3.3 | 0 | 0 | 0 | 0 | 0 |
| T236V | 42 | 6.5 | 17 | 0 | 3.3 | 0 | 0 | 0 | 0 | 0 |
| T236K | 16 | 0 | 17 | 0 | 3.3 | 0 | 0 | 0 | 0 | 0 |
| T236Q | 16 | 0 | 17 | 0 | 3.3 | 0 | 0 | 0 | 0 | 0 |
| T236E | 5.6 | 0 | 17 | 0 | 3.3 | 0 | 0 | 0 | 0 | 0 |
| T236D | 0 | 0 | 17 | 0 | 3.3 | 0 | 0 | 0 | 0 | 0 |
| R239A | 5.6 | 0 | 2.2 | 0 | 3.3 | 0 | 0 | 0 | 0 | 0 |
| R239K | 5.6 | 0 | 6.3 | 0 | 3.3 | 0 | 0 | 0 | 0 | 0 |
| R239E | 5.6 | 0 | 2.2 | 0 | 3.3 | 0 | 0 | 0 | 0 | 0 |
| R239D | 5.6 | 0 | 0.4 | 0 | 3.3 | 0 | 0 | 0 | 0 | 0 |
| I241A | 0 | 0 | 7.3 | 0 | 3.3 | 0 | 0 | 0 | 0 | 0 |
| I241E | 0 | 0 | 19 | 0 | 3.3 | 0 | 0 | 0 | 0 | 0 |
| I241L | 5.6 | 0 | 18 | 0 | 3.3 | 0 | 0 | 0 | 0 | 0 |
| I241T | 0 | 0 | 11 | 0 | 3.3 | 0 | 0 | 0 | 0 | 0 |
| I241V | 0 | 0 | 4.2 | 0 | 3.3 | 0 | 0 | 0 | 0 | 0 |
| I241Q | 0 | 0 | 11 | 0 | 3.3 | 0 | 0 | 0 | 0 | 0 |
| I241Y | 0 | 0 | 24 | 0 | 3.3 | 0 | 0 | 0 | 0 | 0 |
| E246Q | 5.6 | 0 | 41 | 0 | 3.3 | 0 | 0 | 0 | 0 | 0 |
| E246K | 5.6 | 0 | 39 | 0 | 3.3 | 0 | 0 | 0 | 0 | 0 |
| L248A | 5.6 | 0 | 17 | 0 | 3.3 | 0 | 0 | 0 | 0 | 0 |
| L248E | 5.6 | 0 | 17 | 0 | 3.3 | 0 | 0 | 0 | 0 | 0 |
| L248K | 5.6 | 0 | 17 | 0 | 3.3 | 0 | 0 | 0 | 0 | 0 |
| L248N | 5.6 | 0 | 17 | 0 | 3.3 | 0 | 0 | 0 | 0 | 0 |
| L248Q | 5.6 | 0 | 17 | 0 | 3.3 | 0 | 0 | 0 | 0 | 0 |
| L248R | 5.6 | 0 | 17 | 0 | 3.3 | 0 | 0 | 0 | 0 | 0 |
| L248S | 5.6 | 0 | 17 | 0 | 3.3 | 0 | 0 | 0 | 0 | 0 |
| L248Y | 5.6 | 0 | 17 | 0 | 3.3 | 0 | 0 | 0 | 0 | 0 |
| L248F | 5.6 | 0 | 17 | 0 | 3.3 | 0 | 0 | 0 | 0 | 0 |
| N250A | 5.6 | 0 | 17 | 0 | 3.3 | 0 | 0 | 0 | 0 | 0 |
| N250S | 5.6 | 0 | 17 | 0 | 3.3 | 0 | 0 | 0 | 0 | 0 |
| N250D | 5.6 | 0 | 17 | 0 | 3.3 | 0 | 0 | 0 | 0 | 0 |
| N250Y | 5.6 | 0 | 17 | 8.3 | 3.3 | 0 | 0 | 0 | 0 | 0 |
| P272N | 5.6 | 0 | 17 | 0 | 1 | 0 | 8.6 | 0 | 0 | 0 |
| P272D | 5.6 | 0 | 17 | 0 | 0 | 0 | 0 | 0 | 0 | 0 |
| P272S | 5.6 | 0 | 17 | 0 | 1 | 0 | 0 | 0 | 0 | 0 |
| P272A | 5.6 | 0 | 17 | 0 | 0.4 | 0 | 0 | 0 | 0 | 0 |
| R273A | 5.6 | 0 | 17 | 0 | 0 | 0 | 0 | 0 | 0 | 0 |
| R273K | 5.6 | 0 | 17 | 0 | 0 | 0 | 0 | 0 | 0 | 0 |
| R273L | 5.6 | 0 | 17 | 0 | 1.1 | 0 | 0 | 0 | 0 | 0 |
| R273E | 5.6 | 0 | 17 | 0 | 0 | 0 | 0 | 0 | 0 | 0 |
| R273H | 5.6 | 0 | 17 | 0 | 0 | 0 | 0 | 0 | 0 | 0 |
| E274A | 5.6 | 0 | 17 | 0 | 3.3 | 0 | 0 | 0 | 0 | 0 |
| E274L | 5.6 | 0 | 17 | 0 | 3.3 | 0 | 0 | 0 | 0 | 0 |
| E274Q | 5.6 | 0 | 17 | 0 | 3.3 | 0 | 0 | 0 | 0 | 0 |
| E274T | 5.6 | 0 | 17 | 0 | 3.3 | 5.3 | 0 | 0 | 0 | 0 |
| E274K | 5.6 | 0 | 17 | 0 | 3.3 | 0 | 0 | 0 | 0 | 0 |
| E274R | 5.6 | 0 | 17 | 0 | 3.3 | 0 | 1 | 0 | 0 | 0 |
| E274D | 5.6 | 0 | 17 | 0 | 3.3 | 0 | 5.2 | 0 | 0 | 0 |
| E274I | 5.6 | 0 | 17 | 0 | 3.3 | 0 | 0 | 0 | 0 | 0 |
| E274F | 5.6 | 0 | 17 | 0 | 3.3 | 0 | 0 | 3.4 | 0 | 0 |
| N275D | 5.6 | 0 | 17 | 0 | 2.6 | 0 | 0 | 0 | 0 | 0 |
| N275E | 5.6 | 0 | 17 | 0 | 8.4 | 0 | 0 | 0 | 0 | 0 |
| N275R | 5.6 | 0 | 17 | 0 | 19 | 0 | 0 | 0 | 0 | 0 |
| N275S | 5.6 | 0 | 17 | 0 | 31 | 0 | 0 | 0 | 0 | 0 |
| Q277H | 5.6 | 0 | 17 | 0 | 3.3 | 0 | 0 | 0 | 0 | 0 |
| Q277K | 5.6 | 0 | 17 | 0 | 3.3 | 0 | 0 | 0 | 0 | 0 |
| Q277S | 5.6 | 0 | 17 | 0 | 3.3 | 0 | 0 | 0 | 0 | 0 |
| Q277R | 5.6 | 0 | 17 | 0 | 3.3 | 0 | 0 | 0 | 0 | 0 |
| Q277E | 5.6 | 0 | 17 | 0 | 3.3 | 0 | 0 | 0 | 0 | 0 |
| S279R | 5.6 | 0 | 17 | 0 | 3.3 | 0 | 0 | 0 | 13 | 0 |
| S279E | 5.6 | 0 | 17 | 0 | 3.3 | 0 | 0 | 0 | 0 | 0 |
| D281A | 5.6 | 0 | 17 | 0 | 3.3 | 0 | 0 | 0 | 0 | 13 |
| D281E | 5.6 | 0 | 17 | 0 | 3.3 | 0 | 0 | 0 | 0 | 13 |
| D281S | 5.6 | 0 | 17 | 0 | 3.3 | 0 | 0 | 0 | 0 | 13 |
| D281K | 5.6 | 0 | 17 | 0 | 3.3 | 0 | 0 | 0 | 0 | 26 |
| D281R | 5.6 | 0 | 17 | 0 | 3.3 | 0 | 0 | 0 | 0 | 30 |
| D281H | 5.6 | 0 | 17 | 0 | 3.3 | 0 | 0 | 0 | 0 | 26 |
| D281N | 5.6 | 0 | 17 | 0 | 3.3 | 0 | 0 | 0 | 0 | 26 |
| D283A | 5.6 | 0 | 17 | 0 | 3.3 | 0 | 0 | 0 | 13 | 0 |
| D283V | 5.6 | 0 | 17 | 0 | 3.3 | 0 | 0 | 0 | 26 | 0 |
| D283K | 5.6 | 0 | 17 | 0 | 3.3 | 0 | 0 | 0 | 26 | 0 |
| D283R | 5.6 | 0 | 17 | 0 | 3.3 | 0 | 0 | 0 | 26 | 0 |
| D283H | 5.6 | 0 | 17 | 0 | 3.3 | 0 | 0 | 0 | 13 | 0 |
| D283N | 5.6 | 0 | 17 | 0 | 3.3 | 0 | 0 | 0 | 5.2 | 0 |
| D283E | 5.6 | 0 | 17 | 0 | 3.3 | 0 | 0 | 0 | 0 | 0 |

TABLE 12
MHC agretopes in RANKL variants designed for soluble expression,
superagonism, dominant negative inhibition, and competitive inhibition.

| | 215- | 220- | 222- | 235- | 236- | 240- | 241- | 247- | 264- | 270- | 281- | 291- |
|---|---|---|---|---|---|---|---|---|---|---|---|---|
| residues | 223 | 228 | 230 | 243 | 244 | 248 | 249 | 255 | 272 | 278 | 289 | 299 |
| wild type | 5.5 | 0.0 | 15.5 | 3.0 | 1.5 | 4.7 | 35.4 | 10.3 | 0.0 | 9.4 | 0.0 | 0.0 |
| C221S/I247E | 0.0 | 0.0 | 15.5 | 3.0 | 1.5 | 4.7 | 15.2 | 0.0 | 0.0 | 9.4 | 0.0 | 0.0 |
| C221S/I247E/H180E | 0.0 | 0.0 | 15.5 | 3.0 | 1.5 | 4.7 | 15.2 | 0.0 | 0.0 | 9.4 | 0.0 | 0.0 |
| C221S/I247E/K181Q | 0.0 | 0.0 | 15.5 | 3.0 | 1.5 | 4.7 | 15.2 | 0.0 | 0.0 | 9.4 | 0.0 | 0.0 |
| C221S/I247E/K181E | 0.0 | 0.0 | 15.5 | 3.0 | 1.5 | 4.7 | 15.2 | 0.0 | 0.0 | 9.4 | 0.0 | 0.0 |
| C221S/I247E/H189R | 0.0 | 0.0 | 15.5 | 3.0 | 1.5 | 4.7 | 15.2 | 0.0 | 0.0 | 9.4 | 0.0 | 0.0 |
| C221S/I247E/R191Q | 0.0 | 0.0 | 15.5 | 3.0 | 1.5 | 4.7 | 15.2 | 0.0 | 0.0 | 9.4 | 0.0 | 0.0 |
| C221S/I247E/G192A | 0.0 | 0.0 | 15.5 | 3.0 | 1.5 | 4.7 | 15.2 | 0.0 | 0.0 | 9.4 | 0.0 | 0.0 |
| C221S/I247E/S197E | 0.0 | 0.0 | 15.5 | 3.0 | 1.5 | 4.7 | 15.2 | 0.0 | 0.0 | 9.4 | 0.0 | 0.0 |
| C221S/I247E/R223M | 2.9 | 0.0 | 4.5 | 3.0 | 1.5 | 4.7 | 15.2 | 0.0 | 0.0 | 9.4 | 0.0 | 0.0 |
| C221S/I247E/R223E | 1.1 | 0.0 | 0.4 | 3.0 | 1.5 | 4.7 | 15.2 | 0.0 | 0.0 | 9.4 | 0.0 | 0.0 |
| C221S/I247E/R223Q | 1.8 | 0.0 | 4.5 | 3.0 | 1.5 | 4.7 | 15.2 | 0.0 | 0.0 | 9.4 | 0.0 | 0.0 |
| C221S/I247E/H225T | 0.0 | 0.4 | 10.9 | 3.0 | 1.5 | 4.7 | 15.2 | 0.0 | 0.0 | 9.4 | 0.0 | 0.0 |
| C221S/I247E/H225N | 0.0 | 0.0 | 12.3 | 3.0 | 1.5 | 4.7 | 15.2 | 0.0 | 0.0 | 9.4 | 0.0 | 0.0 |
| C221S/I247E/H225E | 0.0 | 0.0 | 19.4 | 3.0 | 1.5 | 4.7 | 15.2 | 0.0 | 0.0 | 9.4 | 0.0 | 0.0 |
| C221S/I247E/H225R | 0.0 | 0.0 | 7.3 | 3.0 | 1.5 | 4.7 | 15.2 | 0.0 | 0.0 | 9.4 | 0.0 | 0.0 |
| C221S/I247E/E226Q | 0.0 | 0.0 | 15.5 | 3.0 | 1.5 | 4.7 | 15.2 | 0.0 | 0.0 | 9.4 | 0.0 | 0.0 |
| C221S/I247E/E226D | 0.0 | 0.0 | 15.5 | 3.0 | 1.5 | 4.7 | 15.2 | 0.0 | 0.0 | 9.4 | 0.0 | 0.0 |
| C221S/I247E/E226R | 0.0 | 0.8 | 15.5 | 3.0 | 1.5 | 4.7 | 15.2 | 0.0 | 0.0 | 9.4 | 0.0 | 0.0 |
| C221S/I247E/T227K | 0.0 | 0.0 | 5.5 | 3.0 | 1.5 | 4.7 | 15.2 | 0.0 | 0.0 | 9.4 | 0.0 | 0.0 |
| C221S/I247E/S228E | 0.0 | 0.0 | 22.8 | 3.0 | 1.5 | 4.7 | 15.2 | 0.0 | 0.0 | 9.4 | 0.0 | 0.0 |
| C221S/I247E/S228H | 0.0 | 0.0 | 30.5 | 3.0 | 1.5 | 4.7 | 15.2 | 0.0 | 0.0 | 9.4 | 0.0 | 0.0 |
| C221S/I247E/Q237T | 0.0 | 0.0 | 15.5 | 3.0 | 0.0 | 4.7 | 15.2 | 0.0 | 0.0 | 9.4 | 0.0 | 0.0 |
| C221S/I247E/Q237K | 0.0 | 0.0 | 15.5 | 3.0 | 0.0 | 4.7 | 15.2 | 0.0 | 0.0 | 9.4 | 0.0 | 0.0 |
| C221S/I247E/Q237E | 0.0 | 0.0 | 15.5 | 0.0 | 0.0 | 4.7 | 15.2 | 0.0 | 0.0 | 9.4 | 0.0 | 0.0 |
| C221S/I247E/K248E | 0.0 | 0.0 | 15.5 | 3.0 | 1.5 | 1.1 | 15.2 | 0.0 | 0.0 | 9.4 | 0.0 | 0.0 |
| C221S/I247E/I249Q | 0.0 | 0.0 | 15.5 | 3.0 | 1.5 | 4.7 | 3.1 | 0.0 | 0.0 | 9.4 | 0.0 | 0.0 |
| C221S/I247E/H253S | 0.0 | 0.0 | 15.5 | 3.0 | 1.5 | 4.7 | 15.2 | 0.0 | 0.0 | 9.4 | 0.0 | 0.0 |
| C221S/I247E/H253T | 0.0 | 0.0 | 15.5 | 3.0 | 1.5 | 4.7 | 15.2 | 0.0 | 0.0 | 9.4 | 0.0 | 0.0 |
| C221S/I247E/S268D | 0.0 | 0.0 | 15.5 | 3.0 | 1.5 | 4.7 | 15.2 | 0.0 | 0.0 | 9.4 | 0.0 | 0.0 |
| C221S/I247E/E269R | 0.0 | 0.0 | 15.5 | 3.0 | 1.5 | 4.7 | 15.2 | 0.0 | 0.0 | 9.4 | 0.0 | 0.0 |
| C221S/I247E/E269T | 0.0 | 0.0 | 15.5 | 3.0 | 1.5 | 4.7 | 15.2 | 0.0 | 0.0 | 9.4 | 0.0 | 0.0 |
| C221S/I247E/E269Q | 0.0 | 0.0 | 15.5 | 3.0 | 1.5 | 4.7 | 15.2 | 0.0 | 0.0 | 9.4 | 0.0 | 0.0 |
| C221S/I247E/E269K | 0.0 | 0.0 | 15.5 | 3.0 | 1.5 | 4.7 | 15.2 | 0.0 | 2.2 | 9.4 | 0.0 | 0.0 |
| C221S/I247E/F270T | 0.0 | 0.0 | 15.5 | 3.0 | 1.5 | 4.7 | 15.2 | 0.0 | 0.0 | 0.0 | 0.0 | 0.0 |
| C221S/I247E/F270K | 0.0 | 0.0 | 15.5 | 3.0 | 1.5 | 4.7 | 15.2 | 0.0 | 0.0 | 0.0 | 0.0 | 0.0 |
| C221S/I247E/F270E | 0.0 | 0.0 | 15.5 | 3.0 | 1.5 | 4.7 | 15.2 | 0.0 | 0.0 | 0.0 | 0.0 | 0.0 |
| C221S/I247E/F270V | 0.0 | 0.0 | 15.5 | 3.0 | 1.5 | 4.7 | 15.2 | 0.0 | 0.0 | 7.6 | 0.0 | 0.0 |
| C221S/I247E/R284E | 0.0 | 0.0 | 15.5 | 3.0 | 1.5 | 4.7 | 15.2 | 0.0 | 0.0 | 9.4 | 7.6 | 0.0 |
| C221S/I247E/S297Q | 0.0 | 0.0 | 15.5 | 3.0 | 1.5 | 4.7 | 15.2 | 0.0 | 0.0 | 9.4 | 0.0 | 6.1 |
| C221S/I247E/D300N | 0.0 | 0.0 | 15.5 | 3.0 | 1.5 | 4.7 | 15.2 | 0.0 | 0.0 | 9.4 | 0.0 | 0.0 |
| C221S/I247E/D302H | 0.0 | 0.0 | 15.5 | 3.0 | 1.5 | 4.7 | 15.2 | 0.0 | 0.0 | 9.4 | 0.0 | 0.0 |
| C221S/I247E/D302E | 0.0 | 0.0 | 15.5 | 3.0 | 1.5 | 4.7 | 15.2 | 0.0 | 0.0 | 9.4 | 0.0 | 0.0 |
| C221S/I247E/E269T/F270T | 0.0 | 0.0 | 15.5 | 3.0 | 1.5 | 4.7 | 15.2 | 0.0 | 0.0 | 0.0 | 0.0 | 0.0 |
| C221S/I247E/H225E/E269K | 0.0 | 0.0 | 19.4 | 3.0 | 1.5 | 4.7 | 15.2 | 0.0 | 2.2 | 9.4 | 0.0 | 0.0 |
| C221S/I247E/H225E/F270T | 0.0 | 0.0 | 19.4 | 3.0 | 1.5 | 4.7 | 15.2 | 0.0 | 0.0 | 0.0 | 0.0 | 0.0 |
| C221S/I247E/H225R/E269K | 0.0 | 0.0 | 7.3 | 3.0 | 1.5 | 4.7 | 15.2 | 0.0 | 2.2 | 9.4 | 0.0 | 0.0 |
| C221S/I247E/H225R/F270T | 0.0 | 0.0 | 7.3 | 3.0 | 1.5 | 4.7 | 15.2 | 0.0 | 0.0 | 0.0 | 0.0 | 0.0 |
| C221S/I247E/R191E/I249R | 0.0 | 0.0 | 15.5 | 3.0 | 1.5 | 4.7 | 0.0 | 0.0 | 0.0 | 9.4 | 0.0 | 0.0 |
| C221S/I247E/G192AH225N | 0.0 | 0.0 | 12.3 | 3.0 | 1.5 | 4.7 | 15.2 | 0.0 | 0.0 | 9.4 | 0.0 | 0.0 |
| C221S/I247E/H225NI249R | 0.0 | 0.0 | 12.3 | 3.0 | 1.5 | 4.7 | 0.0 | 0.0 | 0.0 | 9.4 | 0.0 | 0.0 |
| C221S/I247E/H225RI249R | 0.0 | 0.0 | 7.3 | 3.0 | 1.5 | 4.7 | 0.0 | 0.0 | 0.0 | 9.4 | 0.0 | 0.0 |
| C221S/I247E/E226RI249R | 0.0 | 0.8 | 15.5 | 3.0 | 1.5 | 4.7 | 0.0 | 0.0 | 0.0 | 9.4 | 0.0 | 0.0 |
| C221S/I247E/T227QK248E | 0.0 | 0.0 | 0.0 | 3.0 | 1.5 | 1.1 | 15.2 | 0.0 | 0.0 | 9.4 | 0.0 | 0.0 |
| C221S/I247E/S228EI249R | 0.0 | 0.0 | 22.8 | 3.0 | 1.5 | 4.7 | 0.0 | 0.0 | 0.0 | 9.4 | 0.0 | 0.0 |
| C221S/I247E/D190T/K248E | 0.0 | 0.0 | 15.5 | 3.0 | 1.5 | 1.1 | 15.2 | 0.0 | 0.0 | 9.4 | 0.0 | 0.0 |
| C221S/I247E/D190T/H225R | 0.0 | 0.0 | 7.3 | 3.0 | 1.5 | 4.7 | 15.2 | 0.0 | 0.0 | 9.4 | 0.0 | 0.0 |
| C221S/I247E/D190Q/K248E | 0.0 | 0.0 | 15.5 | 3.0 | 1.5 | 1.1 | 15.2 | 0.0 | 0.0 | 9.4 | 0.0 | 0.0 |
| C221S/I247E/D190Q/H225R | 0.0 | 0.0 | 7.3 | 3.0 | 1.5 | 4.7 | 15.2 | 0.0 | 0.0 | 9.4 | 0.0 | 0.0 |
| C221S/I247E/D190Q/K248E | 0.0 | 0.0 | 15.5 | 3.0 | 1.5 | 1.1 | 15.2 | 0.0 | 0.0 | 9.4 | 0.0 | 0.0 |
| C221S/I247E/G192A/H225R | 0.0 | 0.0 | 7.3 | 3.0 | 1.5 | 4.7 | 15.2 | 0.0 | 0.0 | 9.4 | 0.0 | 0.0 |
| C221S/I247E/D190Q/H225R | 0.0 | 0.0 | 7.3 | 3.0 | 1.5 | 4.7 | 15.2 | 0.0 | 0.0 | 9.4 | 0.0 | 0.0 |
| C221S/I247E/G192A/K248E | 0.0 | 0.0 | 15.5 | 3.0 | 1.5 | 1.1 | 15.2 | 0.0 | 0.0 | 9.4 | 0.0 | 0.0 |
| C221S/I247E/H225E/E226D/ | 0.0 | 0.0 | 19.4 | 3.0 | 1.5 | 4.7 | 15.2 | 0.0 | 0.0 | 0.0 | 0.0 | 0.0 |
| F270T | | | | | | | | | | | | |
| C221S/I247E/R191K/E226R/ | 0.0 | 0.8 | 15.5 | 3.0 | 1.5 | 1.1 | 15.2 | 0.0 | 0.0 | 9.4 | 0.0 | 0.0 |
| K248E | | | | | | | | | | | | |
| C221S/I247E/R191K/E226Q/ | 0.0 | 0.0 | 11.3 | 3.0 | 1.5 | 1.1 | 15.2 | 0.0 | 0.0 | 9.4 | 0.0 | 0.0 |
| K248E | | | | | | | | | | | | |
| C221S/I247E/G192A/K248E/ | 0.0 | 0.0 | 15.5 | 3.0 | 1.5 | 1.1 | 15.2 | 0.0 | 0.0 | 0.0 | 0.0 | 0.0 |
| F270T | | | | | | | | | | | | |
| C221S/I247E/G192A/H225N/ | 0.0 | 0.0 | 12.3 | 3.0 | 1.5 | 4.7 | 0.0 | 0.0 | 0.0 | 9.4 | 0.0 | 0.0 |
| I249R | | | | | | | | | | | | |
| C221S/I247E/G192A/H225R/ | 0.0 | 0.0 | 7.3 | 3.0 | 1.5 | 4.7 | 0.0 | 0.0 | 0.0 | 9.4 | 0.0 | 0.0 |
| I249R | | | | | | | | | | | | |

TABLE 5.D
Comparison of MHC binding agretopes in wild type human
CD40L and designed CD40L variants.

| | 145- | 146- | 152 = | 168- | 169- | 170- | 171- | 175- | 189- | 204- | 205- | 206- | 223- | 229- | 237- | 253- |
|---|---|---|---|---|---|---|---|---|---|---|---|---|---|---|---|---|
| | 153 | 154 | 160 | 176 | 177 | 178 | 179 | 183 | 197 | 212 | 213 | 214 | 231 | 237 | 245 | 261 |
| wt | 18 | 14 | 9 | 10 | 8 | 14 | 19 | 1 | 15 | 5 | 12 | 48 | 18 | 8 | 9 | 13 |
| A123E | 18 | 14 | 9 | 10 | 8 | 14 | 19 | 1 | 15 | 5 | 12 | 48 | 18 | 8 | 9 | 13 |
| H125R | 18 | 14 | 9 | 10 | 8 | 14 | 19 | 1 | 15 | 5 | 12 | 48 | 18 | 8 | 9 | 13 |
| V126A | 18 | 14 | 9 | 10 | 8 | 14 | 19 | 1 | 15 | 5 | 12 | 48 | 18 | 8 | 9 | 13 |
| V126D | 18 | 14 | 9 | 10 | 8 | 14 | 19 | 1 | 15 | 5 | 12 | 48 | 18 | 8 | 9 | 13 |
| W140C | 18 | 14 | 9 | 10 | 8 | 14 | 19 | 1 | 15 | 5 | 12 | 48 | 18 | 8 | 9 | 13 |
| W140G | 18 | 14 | 9 | 10 | 8 | 14 | 19 | 1 | 15 | 5 | 12 | 48 | 18 | 8 | 9 | 13 |
| W140R | 18 | 14 | 9 | 10 | 8 | 14 | 19 | 1 | 15 | 5 | 12 | 48 | 18 | 8 | 9 | 13 |
| W140X | 18 | 14 | 9 | 10 | 8 | 14 | 19 | 1 | 15 | 5 | 12 | 48 | 18 | 8 | 9 | 13 |
| K143E | 18 | 14 | 9 | 10 | 8 | 14 | 19 | 1 | 15 | 5 | 12 | 48 | 18 | 8 | 9 | 13 |
| G144E | 18 | 14 | 9 | 10 | 8 | 14 | 19 | 1 | 15 | 5 | 12 | 48 | 18 | 8 | 9 | 13 |
| T147N | 22 | 32 | 9 | 10 | 8 | 14 | 19 | 1 | 15 | 5 | 12 | 48 | 18 | 8 | 9 | 13 |
| L155P | 18 | 14 | 0 | 10 | 8 | 14 | 19 | 1 | 15 | 5 | 12 | 48 | 18 | 8 | 9 | 13 |
| Y170P | 18 | 14 | 9 | 3 | 0 | 0 | 19 | 1 | 15 | 5 | 12 | 48 | 18 | 8 | 9 | 13 |
| A173D | 18 | 14 | 9 | 0 | 8 | 29 | 2 | 1 | 15 | 5 | 12 | 48 | 18 | 8 | 9 | 13 |
| Q174R | 18 | 14 | 9 | 6 | 35 | 14 | 12 | 1 | 15 | 5 | 12 | 48 | 18 | 8 | 9 | 13 |
| T176I | 18 | 14 | 9 | 34 | 8 | 30 | 9 | 4 | 15 | 5 | 12 | 48 | 18 | 8 | 9 | 13 |
| S184X | 18 | 14 | 9 | 10 | 8 | 14 | 19 | 1 | 15 | 5 | 12 | 48 | 18 | 8 | 9 | 13 |
| Q186X | 18 | 14 | 9 | 10 | 8 | 14 | 19 | 1 | 15 | 5 | 12 | 48 | 18 | 8 | 9 | 13 |
| L195P | 18 | 14 | 9 | 10 | 8 | 14 | 19 | 1 | 0 | 5 | 12 | 48 | 18 | 8 | 9 | 13 |
| R200X | 18 | 14 | 9 | 10 | 8 | 14 | 19 | 1 | 15 | 5 | 12 | 48 | 18 | 8 | 9 | 13 |
| E202X | 18 | 14 | 9 | 10 | 8 | 14 | 19 | 1 | 15 | 5 | 12 | 48 | 18 | 8 | 9 | 13 |
| R203E | 18 | 14 | 9 | 10 | 8 | 14 | 19 | 1 | 15 | 5 | 12 | 48 | 18 | 8 | 9 | 13 |
| R207E | 18 | 14 | 9 | 10 | 8 | 14 | 19 | 1 | 15 | 9 | 1 | 2 | 18 | 8 | 9 | 13 |
| A208D | 18 | 14 | 9 | 10 | 8 | 14 | 19 | 1 | 15 | 5 | 31 | 11 | 18 | 8 | 9 | 13 |
| C218X | 18 | 14 | 9 | 10 | 8 | 14 | 19 | 1 | 15 | 5 | 12 | 48 | 18 | 8 | 9 | 13 |
| Q220X | 18 | 14 | 9 | 10 | 8 | 14 | 19 | 1 | 15 | 5 | 12 | 48 | 18 | 8 | 9 | 13 |
| Q221X | 18 | 14 | 9 | 10 | 8 | 14 | 19 | 1 | 15 | 5 | 12 | 48 | 18 | 8 | 9 | 13 |
| H224Y | 18 | 14 | 9 | 10 | 8 | 14 | 19 | 1 | 15 | 5 | 12 | 48 | 18 | 8 | 9 | 13 |
| G226A | 18 | 14 | 9 | 10 | 8 | 14 | 19 | 1 | 15 | 5 | 12 | 48 | 24 | 8 | 9 | 13 |
| G227V | 18 | 14 | 9 | 10 | 8 | 14 | 19 | 1 | 15 | 5 | 12 | 48 | 18 | 8 | 9 | 13 |
| L231S | 18 | 14 | 9 | 10 | 8 | 14 | 19 | 1 | 15 | 5 | 12 | 48 | 30 | 0 | 9 | 13 |
| Q232X | 18 | 14 | 9 | 10 | 8 | 14 | 19 | 1 | 15 | 5 | 12 | 48 | 18 | 0 | 9 | 13 |
| A235P | 18 | 14 | 9 | 10 | 8 | 14 | 19 | 1 | 15 | 5 | 12 | 48 | 18 | 8 | 9 | 13 |
| S236X | 18 | 14 | 9 | 10 | 8 | 14 | 19 | 1 | 15 | 5 | 12 | 48 | 18 | 0 | 9 | 13 |
| V237E | 18 | 14 | 9 | 10 | 8 | 14 | 19 | 1 | 15 | 5 | 12 | 48 | 18 | 0 | 0 | 13 |
| T254M | 18 | 14 | 9 | 10 | 8 | 14 | 19 | 1 | 15 | 5 | 12 | 48 | 18 | 8 | 9 | 44 |
| G257D | 18 | 14 | 9 | 10 | 8 | 14 | 19 | 1 | 15 | 5 | 12 | 48 | 18 | 8 | 9 | 13 |
| G257S | 18 | 14 | 9 | 10 | 8 | 14 | 19 | 1 | 15 | 5 | 12 | 48 | 18 | 8 | 9 | 13 |
| L258S | 18 | 14 | 9 | 10 | 8 | 14 | 19 | 1 | 15 | 5 | 12 | 48 | 18 | 8 | 9 | 42 |

In a preferred embodiment, variants that do not have any new agretopes (when compared to the wild type human TNF SF member) are developed for therapeutic use. In an especially preferred embodiment, variants in which Iscore is reduced for one or more agretopes relative to wild type human TNF SF member are developed for therapeutic use.

While the foregoing invention has been described above, it will be clear to one skilled in the art that various changes and additional embodiments made be made without departing from the scope of the invention. All references cited herein, including patents, patent applications (provisional, utility and PCT), and publications are entirely incorporated by reference.

(wild type human BAFF,
extra-cellular domain underlined)

SEQUENCE ID 1

MDDSTEREQSRLTSCLKKREEMKLKECVSILPRKESPSVRSSKDGKLLAA
TLLLALLSCCLTVVSFYQVAALQGDLASLRAELQGHHAEKLPAGAGAPKA
GLEEAPAVTAGLKIFEPPAPGEGNSSQNSRNKRAVQGPEETVTQDCLQLI
ADSETPTIQKGSYTFVPWLLSFKRGSALEEKENKILVKETGYFFIYGQVL
YTDKTYAMGHLIQRKKVHVFGDELSLVTLFRCIQNMPETLPNNSCYSAGI
AKLEEGDELQLAIPRENAQISLDGDVTFFGALKLL
(wild type human RANKL,
extra-cellular domain underlined)

SEQUENCE ID 2

MRRASRDYTKYLRGSEEMGGGPGAPHEGPLHAPPPPAPHQPPAASRSMFV
ALLGLGLGQVVCSVALFFYFRAQMDPNRISEDGTHCIYRILRLHENADFQ
DTTLESQDTKLIPDSCRRIKQAFQGAVQKELQHIVGSQHIRAEKAMVDGS
WLDLAKRSKLEAQPFAHLTINATDIPSGSHKVSLSSWYHDRGWAKISNMT
FSNGKLIVNQDGFYYLYANICFRHHETSGDLATEYLQLMVYVTKTSIKIP
SSHTLMKGGSTKYWSGNSEFHFYSINVGGFFKLRSGEEISIEVSNPSLLD
PDQDATYFGAFKVRDID
(wild type human APRIL,
extra-cellular domain underlined)

SEQUENCE ID 3

MPASSPFLLAPKGPPGNMGGPVREPALSVALWLSWGAALGAVACAMALLT
QQTELQSLRREVSRLQGTGGPSQNGEGYPWQSLPEQSSDALEAWENGERS
RKRRAVLTQKQKKQHSVLHLVPINATSKDDSDVTEVMWQPALRRGRGLQA
QGYGVRIQDAGVYLLYSQVLFQDVTFTMGQVVSREGQGRQETLFRCIRSM
PSHPDRAYNSCYSAGVFHLHQGDILSVIIPRARAKLNLSPHGTFLGFVKL
(wild type human CD40L,
extra-cellular domain underlined)

SEQUENCE ID 4

MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL
(wild type human TRAIL,
extra-cellular domain underlined)

SEQUENCE ID 5

MAMMEVQGGPSLGQTCVLIVIFTVLLQSLCVAVTYVYFTNELKQMQDKYS
KSGIACFLKEDDSYWDPNDEESMNSPCWQVKWQLRQLVRKMILRTSEETI
STVQEKQQNISPLVRERGPQRVAAHITGTRGRSNTLSSPNSKNEKALGRK
INSWESSRSGHSFLSNLHLRNGELVIHEKGFYYIYSQTYFRFQEEIKENT
KNDKQMVQYIYKYTSYPDPILLMKSARNSCWSKDAEYGLYSIYQGGIFEL
KENDRIFVSVTNEHLIDMDHEASFFGAFLVG

1. A non-naturally occurring variant TNF Super Family member protein having reduced immunogenicity as compared with a parent protein, wherein said variant protein comprises at least two amino acid modifications; and, wherein said parent protein is selected from the group consisting of human BAFF (SEQ ID NO: 1), human RANKL (SEQ ID NO:2), human APRIL (SEQ ID NO:3), human CD40L (SEQ ID NO:4), and human TRAIL (SEQ ID NO 5).

2. A variant protein of claim 1 wherein said parent protein is human BAFF.

3. A variant protein of claim 2 wherein at least one amino acid modification is made to the group consisting of Agretope A1 (163-171), Agretope A2 (168-176), Agretope A3 (169-177), Agretope A4 (185-193), Agretope A5 (186-194), Agretope A6 (192-200), Agretope A7 (193-201), Agretope A8 (194-202), Agretope A9 (200-208), Agretope A10 (212-220), Agretope A11 (219-227), Agretope A12 (226-234), Agretope A13 (227-235), Agretope A14 (230-238), Agretope A15 (259-267), and Agretope A16 (276-284).

4. A variant protein of claim 2, wherein at least one amino acid modification is made to the group consisting of positions 192, 194, 195, 200, 203, 205, 206, 208, 212, 213, 214, 215, 217, 218, 220, 226, 227, 229, 231, 234, 276, 277, 278, 279, 281, 282, and 284; and, wherein the possible modifications at position 192 are selected from the group consisting of E, D, Q, and K; wherein the possible modification at position 194 is H; wherein the possible modifications at position 195 are selected from the group consisting of L, T, N, and D; wherein the possible modifications at position 200 are selected from the group consisting of E, Q, K, and N; wherein the possible modification at position 203 is A; wherein the possible modifications at position 205 are selected from the group consisting of Q, N, E, S, D, and G; wherein the possible modifications at position 206 are selected from the group consisting of K, W, R, D, and H; wherein the possible modifications at position 208 are selected from the group consisting of A, T, G, R, K, and E; wherein the possible modifications at position 212 are selected from the group consisting of Q, E, T, N, and D; wherein the possible modifications at position 213 are selected from the group consisting of E, D, and A; wherein the possible modifications at position 214 are selected from the group consisting of N and E; wherein the possible modifications at position 215 are selected from the group consisting of L, E, I, V, R, Q, A, N, T, D, G, H, and W; wherein the possible modifications at position 217 are selected from the group consisting of S, T, Q, N, I, Y, D, K, and E; wherein the possible modifications at position 218 are selected from the group consisting of G, S, D, N, T, Q, K, and E; wherein the possible modifications at position 220 are selected from the group consisting of A, G, T, N, E, D, H, P, and W; wherein the possible modifications at position 226 are selected from the group consisting of E, T, N, D, Q, A, S, Y, K, H, R, F, G, and P; wherein the possible modifications at position 227 are selected from the group consisting of K, L, E, D, Q, T, W, N, A, and H; wherein the possible modifications at position 229 are selected from the group consisting of Q, E, N, T, K, D, and A; wherein the possible modifications at position 231 are selected from the group consisting of M, L, Q, A, E, G, and D; wherein the possible modifications at position 234 are selected from the group consisting of K, R, E, A, D, and G; wherein the possible modifications at position 276 are selected from the group consisting of T, S, A, and N; wherein the possible modification at position 277 is S; wherein the possible modifications at position 278 are selected from the group consisting of H and K; wherein the possible modifications at position 279 are selected from the group consisting of Y and H; wherein the possible modification at position 281 is G; wherein the possible modifications at position 282 are selected from the group consisting of Q, M, K, E, T, N, A, and D; and, wherein the possible modifications at position 284 are selected from the group consisting of E, V, N, M, Q, and T.

5. A variant protein of claim 2 wherein at least one amino acid modification is made to the group consisting of positions 192, 195. 200, 205, 212, 213, 217, 226, 227, 231, 276, 282 and 284; and wherein the possible modifications at position 192 are selected from the group consisting of E, D, Q, and K; wherein the possible modification at position 195 is D; wherein the possible modifications at position 200 are selected from the group consisting of E, Q, K, and N; wherein the possible modifications at position 205 are selected from the group consisting of E and D; wherein the possible modifications at position 212 are selected from the group consisting of Q, E, T, N, and D; wherein the possible modification at position 213 is D; wherein the possible modifications at position 217 are selected from the group consisting of Y, D, and E; wherein the possible modifications at position 226 are selected from the group consisting of E, T, N, D, Q, A, S, K, H, R, G, and P; wherein the possible modification at position 227 is D; wherein the possible modification at position 231 is E; wherein the possible modifications at position 276 are selected from the group consisting of T, S, A, and N; wherein the possible modifications at position 282 are selected from the group consisting of K, E, and D; wherein the possible modification at position 284 is E.

6. A variant protein of claim 1 wherein said parent protein is human RANKL.

7. A variant protein of claim 6 wherein at least one amino acid modification is made to the group consisting of Agretope B1 (193-201), Agretope B2 (207-215), Agretope B3 (213-221), Agretope B4 (214-222), Agretope B5 (215-223), Agretope B6 (222-230), Agretope B7 (235-243), Agretope B8 (236-244), Agretope B9 (238-246), Agretope B10 (239-247), Agretope B11 (240-248), Agretope B12 (241-249), Agretope B13 (247-255), Agretope B14 (270-278), Agretope B15 (277-285), Agretope B16 (280-288), Agretope B17 (289-297), and, Agretope B18 (308-316).

8. A variant protein of claim 6, wherein at least one amino acid modification is made to the group consisting of positions 213, 214, 215, 216, 218, 219, 220, 222, 239, 240, 241, 242, 243, 245, 246, 247, 249, 277, 279, 280, 282, 283, 285, 289, 290, 291, 292, 294, 295, and 297; and, wherein the possible modifications at position 213 are selected from the group consisting of H, M, E, T, D, A, Q, N, S, G, and L; wherein the possible modification at position 214 is F; wherein the possible modification at position 215 is H; wherein the possible modifications at position 216 are selected from the group consisting of V, E, N, Q, T, and D; wherein the possible modification at position 218 is G; wherein the possible modifications at position 219 are selected from the group consisting of K, H, T, Q, D, and A; wherein the possible modifications at position 220 are selected from the group consisting of S, A, G, N, L, E, D, M, Q, and H; wherein the possible modifications at position 222 are selected from the group consisting of Y, H, K, and M; wherein the possible modifications at position 239 are selected from the group consisting of T, E, K, N, A, S, D, and H; wherein the possible modifications at position 240 are selected from the group consisting of N, T, E, A, D, S, K, and G; wherein the possible modifications at position 241 are selected from the group consisting of H, E, T, D, N, A, and Q; wherein the possible modifications at position 242 are selected from the group consisting of T, K, E, A, Q, N, and D; wherein the possible modification at position 243 is E; wherein the possible modifications at position 245 are selected from the group consisting of H, K, E, D, and G; wherein the possible modifications at position 246 are selected from the group consisting of N, D, T, E, R, G, and P; wherein the possible modifications at position 247 are selected from the group consisting of A, S, N, E, T, G, D, Q, H, R, K, V, Y, and W; wherein the possible modifications at position 249 are selected from the group consisting of E, N, D, L, H, S, Y, T, A, K, Q, F, R, V, G, M, W, and P; wherein the possible modifications at position 277 are selected from the group consisting of N, D, A, and P; wherein the possible modification at position 279 is A; wherein the possible modifications at position 280 are selected from the group consisting of Y and T; wherein the possible modifications at position 282 are selected from the group consisting of E, Q, A, D, T, S, H, and G; wherein the possible modifications at position 283 are selected from the group consisting of E, N, H, Q, D, T, S, and A; wherein the possible modifications at position 285 are selected from the group consisting of A, E, D, Q, G, H, and W; wherein the possible modifications at position 289 are selected from the group consisting of T, K, N, A, and S; wherein the possible modification at position 290 is D; wherein the possible modifications at position 291 are selected from the group consisting of V, P, Q, T, E, K, N, and A; wherein the possible modifications at position 292 are selected from the group consisting of A, S, and G; wherein the possible modifications at position 294 are selected from the group consisting of K, Q, R, E, I, A, and H; wherein the possible modifications at position 295 are selected from the group consisting of H, E, Q, L, T, K, R, D, A, and S; wherein the possible modifications at position 297 are selected from the group consisting of K, R, A, D, and G.

9. A variant protein of claim 6, wherein at least one amino acid modifications is selected from a group consisting of positions 213, 239, 240, 241, 242, 246, 247, 277, 282, 283, 285, 289, 291, 294 and 297; and, wherein the possible modifications at position 213 are selected from the group consisting of H, E, T, D, A, Q, N, S, and G; wherein the possible modifications at position 239 are selected from the group consisting of T, E, K, N, A, S, D, and H; wherein the possible modifications at position 240 are selected from the group consisting of T, E, A, D, S, and G; wherein the possible modifications at position 241 are selected from the group consisting of H, E, D, A, and Q; wherein the possible modification at position 242 is D; wherein the possible modifications at position 246 are selected from the group consisting of E; wherein the possible modifications at position 247 are selected from the group consisting of W and D; wherein the possible modifications at position 277 are selected from the group consisting of N, D, A, and P; wherein the possible modifications at position 282 is E; wherein the possible modification at position 283 is D; wherein the possible modifications at position 285 are selected from the group consisting of E and D; wherein the possible modifications at position 289 are selected from the group consisting of T, K, N, A, and S; wherein the possible modification at position 291 is E; wherein the possible modifications at position 294 are selected from the group consisting of K, Q, R, E, and H; and, wherein the possible modification at position 297 is D.

10. A variant protein of claim 1 wherein said parent protein is human APRIL.

11. A variant protein of claim 10 wherein at least one amino acid modification is made to the group consisting of Agretope C1 (117-125), Agretope C2 (120-128), Agretope C3 (121-129), Agretope C4 (138-146), Agretope C5 (142-150), Agretope C6 (155-163), Agretope C7 (162-170), Agretope C8 (163-171), Agretope C9 (164-172), Agretope C10 (170-178), Agretope C11 (194-202), Agretope C12 (197-205), Agretope C13 (217-225), Agretope C14 (227-235), Agretope C15 (228-236), Agretope C16 (236-244), and, Agretope C17 (238-246).

12. A variant protein of claim 10, wherein at least one amino acid modification is made to the group consisting ofpositions 142, 143, 144, 147, 148, 150, 164, 165, 167, 169, 170, 172, 173, 175, 176, 178, 194, 195, 197, 199, 200, and 202; and, wherein the possible modifications at position 142 are selected from the group consisting of F, Y, E, H, K, T, N, Q, A, D, S, W, and G; wherein the possible modifications at position 143 are selected from the group consisting of N, P, Q, T, A, E, S, D, M, G, H, F, Y, and W; wherein the possible modifications at position 144 are selected from the group consisting of V, E, S, T, N, Q, A, D, H, G, K, W, and P; wherein the possible modifications at position 147 are selected from the group consisting of P, A, E, and D; wherein the possible modifications at position 148 are selected from the group consisting of P, K, N, E, D, Q, A, and S; wherein the possible modifications at position 150 are selected from the group consisting of S, P, T, G, and K; wherein the possible modifications at position 164 are selected from the group consisting of Y, F, K, H, E, Q, A, N, D, and S; wherein the possible modifications at position 165 are selected from the group consisting of N, A, D, E, and G; wherein the possible modifications at position 167 are selected from the group consisting of T, K, V, and E; wherein the possible modifications at position 169 are selected from the group consisting of S, G, and D; wherein the possible modifications at position 170 are selected from the group consisting of F, D, H, Y, T, A, N, Q, E, S, and G; wherein the possible modifications at position 172 are selected from the group consisting of E, D, T, A, K, N, and G; wherein the possible modifications at position 173 are selected from the group consisting of N, E, and T; wherein the possible modifications at position 175 are selected from the group consisting of D, S, E, G, and Y; wherein the possible modifications at position 176 are selected from the group consisting of D, K, H, R, and W; wherein the possible modifications at position 178 are selected from the group consisting of K, Q, T, E, N, and A; wherein the possible modifications at position 194 are selected from the group consisting of A, S, T, N, D, H, and G; wherein the possible modifications at position 195 are selected from the group consisting of V, M, Q, E, T, I, K, N, D, A, S, G, and L; wherein the possible modifications at position 197 are selected from the group consisting of E, V, D, T, H, A, Q, S, N, G, K, and R; wherein the possible modifications at position 199 are selected from the group consisting of W, H, and G; wherein the possible modifications at position 200 are selected from the group consisting of N, Q, S, D, K, A, E, T, and G; wherein the possible modifications at position 202 are selected from the group consisting of E, D, G, Q, N, H, W, and Y.

13. A variant protein of claim 10, wherein at least one amino acid modification is made to the group consisting of positions 142, 143, 144, 148, 150, 164, 165, 169, 170, 172, 173, 175, and 194; and wherein the possible modifications at position 142 are selected from the group consisting of E, H, K, T, N, Q, A, D, S, and G; wherein the possible modifications at position 143 are selected from the group consisting of P, T, A, E, S, D, G, and W; wherein the possible modifications at position 144 are selected from the group consisting of E and D; wherein the possible modification at position 148 is D; wherein the possible modification at position 150 is K; wherein the possible modifications at position 164 are selected from the group consisting of K, H, Q, A, N, D, and S; wherein the possible modification at position 165 is D; wherein the possible modifications at position 169 are selected from the group consisting of G and D; wherein the possible modifications at position 170 are selected from the group consisting of D, H, T, A, N, Q, E, S, and G; wherein the possible modification at position 172 is N; wherein the possible modification at position 173 is E; wherein the possible modifications at position 175 are selected from the group consisting of D, E, and Y; and, wherein the possible modifications at position 194 are selected from the group consisting of A, S, T, N, D, H, and G.

14. A variant protein of claim 1 wherein said parent protein is human CD40L, and, wherein at least one amino acid modification is made to the group consisting of Agretope D1 (145-153), Agretope D2 (146-154), Agretope D3 (152-160), Agretope D4 (168-176), Agretope D5 (169-177), Agretope D6 (170-178), Agretope D7 (171-179), Agretope D8 (175-183), Agretope D9 (189-197), Agretope D10 (204-212), Agretope D11 (205-213), Agretope D12 (206-214), Agretope D13 (223-231), Agretope D14 (229-237), Agretope D15 (237-245), and, Agretope D16 (253-261).

15. A variant protein of claim 14, wherein said at least one amino acid modification is made from the group consisting of positions 206, 207, 209, 211, 212 and 214; wherein the possible modifications at position 206 are selected from the group consisting of E, N, D, A, T; wherein the possible modifications at position 207 are selected from the group consisting of Q, N, K, S, D, E, A, T; wherein the possible modification at position 209 is G; wherein the possible modifications at position 211 are selected from the group consisting of D, E, Y, F, G, K, R; wherein the possible modifications at position 212 are selected from the group consisting of F, N, D; and, wherein the possible modifications at position 214 are selected from the group consisting of P, E, D, N, A, H, Q, V, T, K, R, G, I, M, L, W, Y, F.

16. A variant protein of claim 14, wherein said at least one amino acid modification is made from the group consisting of positions 206, 207, and 211; and wherein the possible modifications at position 206 are selected from the group consisting of E, N, D, A, T; wherein the possible modification at position 207 is D; and, wherein the possible modifications at position 211 are selected from the group consisting of D, E, Y, F.

17. A variant protein of claim 1, wherein said parent protein is human TRAIL.

18. A variant protein of claim 17, wherein at least one amino acid modification is made to the group consisting of Agretope E1 (151-159), Agretope E2 (174-182), Agretope E3 (181-189), Agretope E4 (182-190), Agretope E5 (183-191), Agretope E6 (206-214), Agretope E7 (207-215), Agretope E8 (209-217), Agretope E9 (220-228), Agretope E10 (221-229), Agretope E11 (223-231), Agretope E12 (237-245), Agretope E13 (240-248), Agretope E14 (256-264), and, Agretope E15 (257-265).

19. A variant protein of claim 17, wherein at least one amino acid modification is made to the group consisting of 174, 175, 176, 177, 179, 207, 208, 209, 210, 212, 213, 215, 221, 222, 223, 224, 226, 227, 229, 256, 257, 258, 259, 260, 261, 262, 263, 264, and 265; and, wherein the possible modifications at position 174 are selected from the group consisting of E and Q; wherein the possible modifications at position 175 are selected from the group consisting of I, T, Q, E, S, K, D, A, N, and G; wherein the possible modifications at position 176 are selected from the group consisting of V, T, L, and N; wherein the possible modifications at position 177 are selected from the group consisting of E, N, D, T, and W; wherein the possible modifications at position 179 are selected from the group consisting of P, S, E, Q, G, N, D, H, and A; wherein the possible modifications at position 207 are selected from the group consisting of N, T, Q, D, A, E, K, and S; wherein the possible modifications at position 208 are selected from the group consisting of E, D, A, and T; wherein the possible modifications at position 209 are selected from the group consisting of H, K, and E; wherein the possible modifications at position 210 are selected from the group consisting of Q, L, T, N, E, and K; wherein the possible modification at position 212 is G; wherein the possible modifications at position 213 are selected from the group consisting of H and K; wherein the possible modifications at position 215 are selected from the group consisting of E, D, and T; wherein the possible modifications at position 221 are selected from the group consisting of P, N, K, A, and T; wherein the possible modifications at position 222 are selected from the group consisting of E and T; wherein the possible modification at position 223 is Q; wherein the possible modifications at position 224 are selected from the group consisting of A, T, S, E, and G; wherein the possible modification at position 226 is G; wherein the possible modifications at position 227 are selected from the group consisting of Q, E, K, D, A, G, Y, and F; wherein the possible modifications at position 229 are selected from the group consisting of A, G, and T; wherein the possible modifications at position 256 are selected from the group consisting of T, Q, A, K, and N; wherein the possible modification at position 257 is H; wherein the possible modifications at position 258 are selected from the group consisting of P, T, N, S, and A; wherein the possible modifications at position 259 are selected from the group consisting of A and G; wherein the possible modifications at position 260 are selected from the group consisting of D, A, and N; wherein the possible modifications at position 261 are selected from the group consisting of S, E, N, and Q; wherein the possible modifications at position 262 are selected from the group consisting of H, E, F, D, Y, R, K, Q, and W; wherein the possible modifications at position 263 are selected from the group consisting of P, K, and G; wherein the possible modifications at position 264 are selected from the group consisting of E, N, D, A, G, T, K, V, R, M, L, and P; and, wherein the possible modifications at position 265 are selected from the group consisting of E, K, N, D, Q, Y, H, S, F, A, M, R, and G.

20. A variant protein of claim 17 wherein at least one amino acid modification is made to the group consisting of 174, 175, 179, 207, 208, 215, 221, 256, 257, 258, and 261; and, wherein the possible modifications at position 174 are selected from the group consisting of E and Q; wherein the possible modifications at position 175 are selected from the group consisting of T, S, D, and A; wherein the possible modifications at position 179 are selected from the group consisting of E and D; wherein the possible modifications at position 207 are selected from the group consisting of N, T, Q, D, A, E, K, and S; wherein the possible modification at position 208 is D; wherein the possible modification at position 215 is D; wherein the possible modifications at position 221 are selected from the group consisting of P, N, K, A, and T; wherein the possible modifications at position 256 are selected from the group consisting of T, Q, A, K, and N; wherein the possible modification at position 257 is H; wherein the possible modifications at position 258 are selected from the group consisting of P, T, S, and A; and, wherein the possible modifications at position 261 are selected from the group consisting of E and Q;.


SEQUENCE LISTING (772 sequences)

SEQ ID NO: 1 - protein, 285 aa, Homo sapiens
MDDSTEREQSRLTSCLKKREEMKLKECVSILPRKESPSVRSSKDGKLLAATLLLALLSCCLTVVSFYQVAALQGDLASLRAELQGHHAEKLPAGAGAPKAGLEEAPAVTAGLKIFEPPAPGEGNSSQNSRNKRAVQGPEETVTQDCLQLIADSETPTIQKGSYTFVPWLLSFKRGSALEEKENKILVKETGYFFIYGQVLYTDKTYAMGHLIQRKKVHVFGDELSLVTLFRCIQNMPETLPNNSCYSAGIAKLEEGDELQLAIPRENAQISLDGDVTFFGALKLL

SEQ ID NO: 2 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 3 - protein, 250 aa, Homo sapiens [sequence omitted]

SEQ ID NO: 4 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 5 - protein, 281 aa, Homo sapiens [sequence omitted]

SEQ ID NO: 6 - protein, 9 aa, Homo sapiens
YTFVPWLLS

SEQ ID NO: 7 - protein, 9 aa, Homo sapiens
WLLSFKRGS

SEQ ID NO: 8 - protein, 9 aa, Homo sapiens
LLSFKRGSA

SEQ ID NO: 9 - protein, 9 aa, Homo sapiens
ILVKETGYF

SEQ ID NO: 10 - protein, 9 aa, Homo sapiens
LVKETGYFF

SEQ ID NO: 11 - protein, 9 aa, Homo sapiens
YFFIYGQVL

SEQ ID NO: 12 - protein, 9 aa, Homo sapiens
FFIYGQVLY

SEQ ID NO: 13 - protein, 9 aa, Homo sapiens
FIYGQVLYT

SEQ ID NO: 14 - protein, 9 aa, Homo sapiens
LYTDKTYAM

SEQ ID NO: 15 - protein, 9 aa, Homo sapiens
IQRKKVHVF

SEQ ID NO: 16 - protein, 9 aa, Homo sapiens
VFGDELSLV

SEQ ID NO: 17 - protein, 9 aa, Homo sapiens
LVTLFRCIQ

SEQ ID NO: 18 - protein, 9 aa, Homo sapiens
VTLFRCIQN

SEQ ID NO: 19 - protein, 8 aa, Homo sapiens
FRCIQNMP

SEQ ID NO: 20 - protein, 9 aa, Homo sapiens
LQLAIPREN

SEQ ID NO: 21 - protein, 9 aa, Homo sapiens
VTFFGALKL

SEQ ID NO: 22 - protein, 9 aa, Homo sapiens
WAKISNMTF

SEQ ID NO: 23 - protein, 9 aa, Homo sapiens
IVNQDGFYY

SEQ ID NO: 24 - protein, 9 aa, Homo sapiens
FYYLYANIC

SEQ ID NO: 25 - protein, 9 aa, Homo sapiens
YYLYANICF

SEQ ID NO: 26 - protein, 9 aa, Homo sapiens
YLYANICFR

SEQ ID NO: 27 - protein, 9 aa, Homo sapiens
FRHHETSGD

SEQ ID NO: 28 - protein, 9 aa, Homo sapiens
YLQLMVYVT

SEQ ID NO: 29 - protein, 9 aa, Homo sapiens
LQLMVYVTK

SEQ ID NO: 30 - protein, 9 aa, Homo sapiens
LMVYVTKTS

SEQ ID NO: 31 - protein, 9 aa, Homo sapiens
MVYVTKTSI

SEQ ID NO: 32 - protein, 9 aa, Homo sapiens
VYVTKTSIK

SEQ ID NO: 33 - protein, 9 aa, Homo sapiens
YVTKTSIKI

SEQ ID NO: 34 - protein, 9 aa, Homo sapiens
IKIPSSHTL

SEQ ID NO: 35 - protein, 9 aa, Homo sapiens
FHFYSINVG

SEQ ID NO: 36 - protein, 9 aa, Homo sapiens
VGGFFKLRS

SEQ ID NO: 37 - protein, 9 aa, Homo sapiens
FFKLRSGEE

SEQ ID NO: 38 - protein, 9 aa, Homo sapiens
ISIEVSNPS

SEQ ID NO: 39 - protein, 9 aa, Homo sapiens
FGAFKVRDI

SEQ ID NO: 40 - protein, 9 aa, Homo sapiens
VLHLVPINA

SEQ ID NO: 41 - protein, 9 aa, Homo sapiens
LVPINATSK

SEQ ID NO: 42 - protein, 9 aa, Homo sapiens
VPINATSKD

SEQ ID NO: 43 - protein, 9 aa, Homo sapiens
WQPALRRGR

SEQ ID NO: 44 - protein, 9 aa, Homo sapiens
LRRGRGLQA

SEQ ID NO: 45 - protein, 9 aa, Homo sapiens
VRIQDAGVY

SEQ ID NO: 46 - protein, 9 aa, Homo sapiens
VYLLYSQVL

SEQ ID NO: 47 - protein, 9 aa, Homo sapiens
YLLYSQVLF

SEQ ID NO: 48 - protein, 9 aa, Homo sapiens
LLYSQVLFQ

SEQ ID NO: 49 - protein, 9 aa, Homo sapiens
LFQDVTFTM

SEQ ID NO: 50 - protein, 9 aa, Homo sapiens
FRCIRSMPS

SEQ ID NO: 51 - protein, 9 aa, Homo sapiens
IRSMPSHPD

SEQ ID NO: 52 - protein, 9 aa, Homo sapiens
FHLHQGDIL

SEQ ID NO: 53 - protein, 9 aa, Homo sapiens
VIIPRARAK

SEQ ID NO: 54 - protein, 9 aa, Homo sapiens
IIPRARAKL

SEQ ID NO: 55 - protein, 9 aa, Homo sapiens
LNLSPHGTF

SEQ ID NO: 56 - protein, 9 aa, Homo sapiens
LSPHGTFLG

SEQ ID NO: 57 - protein, 9 aa, Homo sapiens
YYTMSNNLV

SEQ ID NO: 58 - protein, 9 aa, Homo sapiens
YTMSNNLVT

SEQ ID NO: 59 - protein, 9 aa, Homo sapiens
LVTLENGKQ

SEQ ID NO: 60 - protein, 9 aa, Homo sapiens
LYYIYAQVT

SEQ ID NO: 61 - protein, 9 aa, Homo sapiens
YYIYAQVTF

SEQ ID NO: 62 - protein, 9 aa, Homo sapiens
YIYAQVTFC

SEQ ID NO: 63 - protein, 9 aa, Homo sapiens
IYAQVTFCS

SEQ ID NO: 64 - protein, 9 aa, Homo sapiens
VTFCSNREA

SEQ ID NO: 65 - protein, 9 aa, Homo sapiens
FIASLCLKS

SEQ ID NO: 66 - protein, 9 aa, Homo sapiens
ILLRAANTH

SEQ ID NO: 67 - protein, 9 aa, Homo sapiens
LLRAANTHS

SEQ ID NO: 68 - protein, 9 aa, Homo sapiens
LRAANTHSS

SEQ ID NO: 69 - protein, 9 aa, Homo sapiens
IHLGGVFEL

SEQ ID NO: 70 - protein, 9 aa, Homo sapiens
FELQPGASV

SEQ ID NO: 71 - protein, 9 aa, Homo sapiens
VFVNVTDPS

SEQ ID NO: 72 - protein, 9 aa, Homo sapiens
FTSFGLLKL

SEQ ID NO: 73 - protein, 9 aa, Homo sapiens
INSWESSRS

SEQ ID NO: 74 - protein, 9 aa, Homo sapiens
LVIHEKGFY

SEQ ID NO: 75 - protein, 9 aa, Homo sapiens
FYYIYSQTY

SEQ ID NO: 76 - protein, 9 aa, Homo sapiens
YYIYSQTYF

SEQ ID NO: 77 - protein, 9 aa, Homo sapiens
YIYSQTYFR

SEQ ID NO: 78 - protein, 9 aa, Homo sapiens
MVQYIYKYT

SEQ ID NO: 79 - protein, 9 aa, Homo sapiens
VQYIYKYTS

SEQ ID NO: 80 - protein, 9 aa, Homo sapiens
YIYKYTSYP

SEQ ID NO: 81 - protein, 9 aa, Homo sapiens
ILLMKSARN

SEQ ID NO: 82 - protein, 9 aa, Homo sapiens
LLMKSARNS

SEQ ID NO: 83 - protein, 9 aa, Homo sapiens
MKSARNSCW

SEQ ID NO: 84 - protein, 9 aa, Homo sapiens
YGLYSIYQG

SEQ ID NO: 85 - protein, 9 aa, Homo sapiens
YSIYQGGIF

SEQ ID NO: 86 - protein, 9 aa, Homo sapiens
IFVSVTNEH

SEQ ID NO: 87 - protein, 9 aa, Homo sapiens
FVSVTNEHL

SEQ ID NO: 88 - protein, 9 aa, Artificial
  Synthetic
WLLSFSRGS

SEQ ID NO: 89 - protein, 9 aa, Artificial
  Synthetic
WLLSFNRGS

SEQ ID NO: 90 - protein, 9 aa, Artificial
  Synthetic
WLLSFERGS

SEQ ID NO: 91 - protein, 9 aa, Artificial
  Synthetic
WLLSFQRGS

SEQ ID NO: 92 - protein, 9 aa, Artificial
  Synthetic
WLLSFKEGS

SEQ ID NO: 93 - protein, 9 aa, Artificial
  Synthetic
WLLSFKQGS

SEQ ID NO: 94 - protein, 9 aa, Artificial
  Synthetic
WLLSFKRGT

SEQ ID NO: 95 - protein, 9 aa, Artificial
  Synthetic
WLLSFKRGN

SEQ ID NO: 96 - protein, 9 aa, Artificial
  Synthetic
WLLSFKRGD

SEQ ID NO: 97 - protein, 9 aa, Artificial
  Synthetic
WLLSFKRGE

SEQ ID NO: 98 - protein, 9 aa, Artificial
  Synthetic
WLLSFKRGQ

SEQ ID NO: 99 - protein, 9 aa, Artificial
  Synthetic
WLLSFKRGK

SEQ ID NO: 100 - protein, 9 aa, Artificial
  Synthetic
WFLSFKNGS

SEQ ID NO: 101 - protein, 9 aa, Artificial
  Synthetic
WFLSFKKGS

SEQ ID NO: 102 - protein, 9 aa, Artificial
  Synthetic
WFLSFKRGA

SEQ ID NO: 103 - protein, 9 aa, Artificial
  Synthetic
WLVGFKRGS

SEQ ID NO: 104 - protein, 9 aa, Artificial
  Synthetic
WLVDFKRGS

SEQ ID NO: 105 - protein, 9 aa, Artificial
  Synthetic
WLVSFKNGS

SEQ ID NO: 106 - protein, 9 aa, Artificial
  Synthetic
WLVSFKHGS

SEQ ID NO: 107 - protein, 9 aa, Artificial
  Synthetic
WLVSFKKGS

SEQ ID NO: 108 - protein, 9 aa, Artificial
  Synthetic
WLVSFKRGA

SEQ ID NO: 109 - protein, 9 aa, Artificial
  Synthetic
WLFSFKNGS

SEQ ID NO: 110 - protein, 9 aa, Artificial
  Synthetic
WLFSFKKGS

SEQ ID NO: 111 - protein, 9 aa, Artificial
  Synthetic
WLFSFKRGA

SEQ ID NO: 112 - protein, 9 aa, Artificial
  Synthetic
WLLTFKNGS

SEQ ID NO: 113 - protein, 9 aa, Artificial
  Synthetic
WLLTFKHGS

SEQ ID NO: 114 - protein, 9 aa, Artificial
  Synthetic
WLLTFKKGS

SEQ ID NO: 115 - protein, 9 aa, Artificial
  Synthetic
WLLTFKRGA

SEQ ID NO: 116 - protein, 9 aa, Artificial
  Synthetic
WLLGFKNGS

SEQ ID NO: 117 - protein, 9 aa, Artificial
  Synthetic
WLLGFKHGS

SEQ ID NO: 118 - protein, 9 aa, Artificial
  Synthetic
WLLGFKKGS

SEQ ID NO: 119 - protein, 9 aa, Artificial
  Synthetic
WLLGFKRGA

SEQ ID NO: 120 - protein, 9 aa, Artificial
  Synthetic
WLLGFKRGG

SEQ ID NO: 121 - protein, 9 aa, Artificial
  Synthetic
WLLDFKRGA

SEQ ID NO: 122 - protein, 9 aa, Artificial
  Synthetic
WLLDFKRGG

SEQ ID NO: 123 - protein, 9 aa, Artificial
  Synthetic
WLLEFKNGS

SEQ ID NO: 124 - protein, 9 aa, Artificial
  Synthetic
WLLEFKHGS

SEQ ID NO: 125 - protein, 9 aa, Artificial
  Synthetic
WLLEFKKGS

SEQ ID NO: 126 - protein, 9 aa, Artificial
  Synthetic
WLLEFKRGA

SEQ ID NO: 127 - protein, 9 aa, Artificial
  Synthetic
WLLSFRNGS

SEQ ID NO: 128 - protein, 9 aa, Artificial
  Synthetic
WLLSFRKGS

SEQ ID NO: 129 - protein, 9 aa, Artificial
  Synthetic
WLLSFRRGA

SEQ ID NO: 130 - protein, 9 aa, Artificial
  Synthetic
WLLSFKNGA

SEQ ID NO: 131 - protein, 9 aa, Artificial
  Synthetic
WLLSFKNGG

SEQ ID NO: 132 - protein, 9 aa, Artificial
  Synthetic
WLLSFKHGA

SEQ ID NO: 133 - protein, 9 aa, Artificial
  Synthetic
WLLSFKHGG

SEQ ID NO: 134 - protein, 9 aa, Artificial
  Synthetic
WLLSFKKGA

SEQ ID NO: 135 - protein, 9 aa, Artificial
  Synthetic
WLLSFKKGG

SEQ ID NO: 136 - protein, 9 aa, Artificial
  Synthetic
WLVTFRRGS

SEQ ID NO: 137 - protein, 9 aa, Artificial
  Synthetic
YWFIYGQVL

SEQ ID NO: 138 - protein, 9 aa, Artificial
  Synthetic
YFWIYGQVL

SEQ ID NO: 139 - protein, 9 aa, Artificial
  Synthetic
YFFVYGQVL

SEQ ID NO: 140 - protein, 9 aa, Artificial
  Synthetic
YFFIYGDVL

SEQ ID NO: 141 - protein, 9 aa, Artificial
  Synthetic
YFFIYGEVL

SEQ ID NO: 142 - protein, 9 aa, Artificial
  Synthetic
YFFIYGQVM

SEQ ID NO: 143 - protein, 9 aa, Artificial
  Synthetic
YFFIYGQVF

SEQ ID NO: 144 - protein, 9 aa, Artificial
  Synthetic
YFFLYNQVL

SEQ ID NO: 145 - protein, 9 aa, Artificial
  Synthetic
YFFFYGRVL

SEQ ID NO: 146 - protein, 9 aa, Artificial
  Synthetic
YFFFYGQVV

SEQ ID NO: 147 - protein, 9 aa, Artificial
  Synthetic
YFFIYSQVV

SEQ ID NO: 148 - protein, 9 aa, Artificial
  Synthetic
YFFIYNQVV

SEQ ID NO: 149 - protein, 9 aa, Artificial
  Synthetic
YFFIYGSVV

SEQ ID NO: 150 - protein, 9 aa, Artificial
  Synthetic
YFFIYGKVV

SEQ ID NO: 151 - protein, 9 aa, Artificial
  Synthetic
FYTDKTYAM

SEQ ID NO: 152 - protein, 9 aa, Artificial
  Synthetic
LWTDKTYAM

SEQ ID NO: 153 - protein, 9 aa, Artificial
  Synthetic
LYTSKTYAM

SEQ ID NO: 154 - protein, 9 aa, Artificial
  Synthetic
LYTNKTYAM

SEQ ID NO: 155 - protein, 9 aa, Artificial
  Synthetic
LYTEKTYAM

SEQ ID NO: 156 - protein, 9 aa, Artificial
  Synthetic
LYTQKTYAM

SEQ ID NO: 157 - protein, 9 aa, Artificial
  Synthetic
LYTDKSYAM

SEQ ID NO: 158 - protein, 9 aa, Artificial
  Synthetic
LYTDKAYAM

SEQ ID NO: 159 - protein, 9 aa, Artificial
  Synthetic
LYTDKNYAM

SEQ ID NO: 160 - protein, 9 aa, Artificial
  Synthetic
LYTDKTHAM

SEQ ID NO: 161 - protein, 9 aa, Artificial
  Synthetic
LYTDKTWAM

SEQ ID NO: 162 - protein, 9 aa, Artificial
  Synthetic
LYTDKTYAQ

SEQ ID NO: 163 - protein, 9 aa, Artificial
  Synthetic
LYTDKTYAI

SEQ ID NO: 164 - protein, 9 aa, Artificial
  Synthetic
LYTDKTYAL

SEQ ID NO: 165 - protein, 9 aa, Artificial
  Synthetic
LYTDKTYAV

SEQ ID NO: 166 - protein, 9 aa, Artificial
  Synthetic
ISRKKVHVF

SEQ ID NO: 167 - protein, 9 aa, Artificial
  Synthetic
IDRKKVHVF

SEQ ID NO: 168 - protein, 9 aa, Artificial
  Synthetic
IERKKVHVF

SEQ ID NO: 169 - protein, 9 aa, Artificial
  Synthetic
IQEKKVHVF

SEQ ID NO: 170 - protein, 9 aa, Artificial
  Synthetic
IQRSKVHVF

SEQ ID NO: 171 - protein, 9 aa, Artificial
  Synthetic
IQREKVHVF

SEQ ID NO: 172 - protein, 9 aa, Artificial
  Synthetic
IQRKKAHVF

SEQ ID NO: 173 - protein, 9 aa, Artificial
  Synthetic
IQRKKMHVF

SEQ ID NO: 174 - protein, 9 aa, Artificial
  Synthetic
IQRKKLHVF

SEQ ID NO: 175 - protein, 9 aa, Artificial
  Synthetic
IQRKKVEVF

SEQ ID NO: 176 - protein, 9 aa, Artificial
  Synthetic
IQRKKVHVL

SEQ ID NO: 177 - protein, 9 aa, Artificial
  Synthetic
IQRKKVHVW

SEQ ID NO: 178 - protein, 9 aa, Artificial
  Synthetic
INQKKVHVF

SEQ ID NO: 179 - protein, 9 aa, Artificial
  Synthetic
INKKKVHVF

SEQ ID NO: 180 - protein, 9 aa, Artificial
  Synthetic
INRRKVHVF

SEQ ID NO: 181 - protein, 9 aa, Artificial
  Synthetic
INRKKIHVF

SEQ ID NO: 182 - protein, 9 aa, Artificial
  Synthetic
INRKKVHVY

SEQ ID NO: 183 - protein, 9 aa, Artificial
  Synthetic
IHQKKVHVF

SEQ ID NO: 184 - protein, 9 aa, Artificial
  Synthetic
IHKKKVHVF

SEQ ID NO: 185 - protein, 9 aa, Artificial
  Synthetic
IHRRKVHVF

SEQ ID NO: 186 - protein, 9 aa, Artificial
  Synthetic
IHRKKIHVF

SEQ ID NO: 187 - protein, 9 aa, Artificial
  Synthetic
IHRKKVHVY

SEQ ID NO: 188 - protein, 9 aa, Artificial
  Synthetic
IQQNKVHVF

SEQ ID NO: 189 - protein, 9 aa, Artificial
  Synthetic
IQQQKVHVF

SEQ ID NO: 190 - protein, 9 aa, Artificial
  Synthetic
IQQRKVHVF

SEQ ID NO: 191 - protein, 9 aa, Artificial
  Synthetic
IQQKKTHVF

SEQ ID NO: 192 - protein, 9 aa, Artificial
  Synthetic
IQQKKIHVF

SEQ ID NO: 193 - protein, 9 aa, Artificial
  Synthetic
IQQKKVHVY

SEQ ID NO: 194 - protein, 9 aa, Artificial
  Synthetic
IQHRKVHVF

SEQ ID NO: 195 - protein, 9 aa, Artificial
  Synthetic
IQHKKTHVF

SEQ ID NO: 196 - protein, 9 aa, Artificial
  Synthetic
IQHKKIHVF

SEQ ID NO: 197 - protein, 9 aa, Artificial
  Synthetic
IQHKKVHVY

SEQ ID NO: 198 - protein, 9 aa, Artificial
  Synthetic
IQKNKVHVF

SEQ ID NO: 199 - protein, 9 aa, Artificial
  Synthetic
IQKQKVHVF

SEQ ID NO: 200 - protein, 9 aa, Artificial
  Synthetic
IQKRKVHVF

SEQ ID NO: 201 - protein, 9 aa, Artificial
  Synthetic
IQKKKTHVF

SEQ ID NO: 202 - protein, 9 aa, Artificial
  Synthetic
IQKKKIHVF

SEQ ID NO: 203 - protein, 9 aa, Artificial
  Synthetic
IQKKKVYVF

SEQ ID NO: 204 - protein, 9 aa, Artificial
  Synthetic
IQKKKVHVI

SEQ ID NO: 205 - protein, 9 aa, Artificial
  Synthetic
IQKKKVHVY

SEQ ID NO: 206 - protein, 9 aa, Artificial
  Synthetic
IQRNKTHVF

SEQ ID NO: 207 - protein, 9 aa, Artificial
  Synthetic
IQRNKIHVF

SEQ ID NO: 208 - protein, 9 aa, Artificial
  Synthetic
IQRNKVHVY

SEQ ID NO: 209 - protein, 9 aa, Artificial
  Synthetic
IQRRKIHVF

SEQ ID NO: 210 - protein, 9 aa, Artificial
  Synthetic
IQRRKVYVF

SEQ ID NO: 211 - protein, 9 aa, Artificial
  Synthetic
IQRRKVHVY

SEQ ID NO: 212 - protein, 9 aa, Artificial
  Synthetic
IQRKKTYVF

SEQ ID NO: 213 - protein, 9 aa, Artificial
  Synthetic
IQRKKTHVY

SEQ ID NO: 214 - protein, 9 aa, Artificial
  Synthetic
IQRKKIQVF

SEQ ID NO: 215 - protein, 9 aa, Artificial
  Synthetic
IQRKKIYVF

SEQ ID NO: 216 - protein, 9 aa, Artificial
  Synthetic
IQRKKIHVY

SEQ ID NO: 217 - protein, 9 aa, Artificial
  Synthetic
IQRKKVYVY

SEQ ID NO: 218 - protein, 9 aa, Artificial
  Synthetic
LTTLFRCIQ

SEQ ID NO: 219 - protein, 9 aa, Artificial
  Synthetic
LATLFRCIQ

SEQ ID NO: 220 - protein, 9 aa, Artificial
  Synthetic
LMTLFRCIQ

SEQ ID NO: 221 - protein, 9 aa, Artificial
  Synthetic
LITLFRCIQ

SEQ ID NO: 222 - protein, 9 aa, Artificial
  Synthetic
LLTLFRCIQ

SEQ ID NO: 223 - protein, 9 aa, Artificial
  Synthetic
LVTLFECIQ

SEQ ID NO: 224 - protein, 9 aa, Artificial
  Synthetic
LVTLFQCIQ

SEQ ID NO: 225 - protein, 9 aa, Artificial
  Synthetic
LVTLFHCIQ

SEQ ID NO: 226 - protein, 9 aa, Artificial
  Synthetic
LVTLFRCIN

SEQ ID NO: 227 - protein, 9 aa, Artificial
  Synthetic
LVTLFRCID

SEQ ID NO: 228 - protein, 9 aa, Artificial
  Synthetic
LVTLFRCIR

SEQ ID NO: 229 - protein, 9 aa, Artificial
  Synthetic
LVTIFRCIE

SEQ ID NO: 230 - protein, 9 aa, Artificial
  Synthetic
LVTIFRCIK

SEQ ID NO: 231 - protein, 9 aa, Artificial
  Synthetic
LVTVFRCIE

SEQ ID NO: 232 - protein, 9 aa, Artificial
  Synthetic
LVTVFRCIH

SEQ ID NO: 233 - protein, 9 aa, Artificial
  Synthetic
LVTVFRCIK

SEQ ID NO: 234 - protein, 9 aa, Artificial
  Synthetic
LVTVFRCIM

SEQ ID NO: 235 - protein, 9 aa, Artificial
  Synthetic
LVTFFRCIE

SEQ ID NO: 236 - protein, 9 aa, Artificial
  Synthetic
VTWFGALKL

SEQ ID NO: 237 - protein, 9 aa, Artificial
  Synthetic
VTFMGALKL

SEQ ID NO: 238 - protein, 9 aa, Artificial
  Synthetic
VTFIGALKL

SEQ ID NO: 239 - protein, 9 aa, Artificial
  Synthetic
VTFWGALKL

SEQ ID NO: 240 - protein, 9 aa, Artificial
  Synthetic
VTFFGAVKL

SEQ ID NO: 241 - protein, 9 aa, Artificial
  Synthetic
VTFFGALKF

SEQ ID NO: 242 - protein, 9 aa, Artificial
  Synthetic
VSFFGVLKL

SEQ ID NO: 243 - protein, 9 aa, Artificial
  Synthetic
VSFFGAIKL

SEQ ID NO: 244 - protein, 9 aa, Artificial
  Synthetic
VSFFGAFKL

SEQ ID NO: 245 - protein, 9 aa, Artificial
  Synthetic
VSFFGALKM

SEQ ID NO: 246 - protein, 9 aa, Artificial
  Synthetic
VTFLGAMKL

SEQ ID NO: 247 - protein, 9 aa, Artificial
  Synthetic
VTFLGALKM

SEQ ID NO: 248 - protein, 9 aa, Artificial
  Synthetic
VTFFGTFKL

SEQ ID NO: 249 - protein, 9 aa, Artificial
  Synthetic
VTFFGVIKL

SEQ ID NO: 250 - protein, 9 aa, Artificial
  Synthetic
VTFFGVFKL

SEQ ID NO: 251 - protein, 9 aa, Artificial
  Synthetic
VTFFGAMKM

SEQ ID NO: 252 - protein, 9 aa, Artificial
  Synthetic
VTFFGAIKM

SEQ ID NO: 253 - protein, 9 aa, Artificial
  Synthetic
VTFFGAIKV

SEQ ID NO: 254 - protein, 9 aa, Artificial
  Synthetic
VTFFGAFKM

SEQ ID NO: 255 - protein, 9 aa, Artificial
  Synthetic
VTFFGAFKV

SEQ ID NO: 256 - protein, 9 aa, Artificial
  Synthetic
FWYLYANIC

SEQ ID NO: 257 - protein, 9 aa, Artificial
  Synthetic
FYWLYANIC

SEQ ID NO: 258 - protein, 9 aa, Artificial
  Synthetic
FYYVYANIC

SEQ ID NO: 259 - protein, 9 aa, Artificial
  Synthetic
FYYLYAGIC

SEQ ID NO: 260 - protein, 9 aa, Artificial
  Synthetic
FYYLYADIC

SEQ ID NO: 261 - protein, 9 aa, Artificial
  Synthetic
FYYLYAEIC

SEQ ID NO: 262 - protein, 9 aa, Artificial
  Synthetic
MYYIYANIC

SEQ ID NO: 263 - protein, 9 aa, Artificial
  Synthetic
MYYFYANIC

SEQ ID NO: 264 - protein, 9 aa, Artificial
  Synthetic
MYYLYGNIC

SEQ ID NO: 265 - protein, 9 aa, Artificial
  Synthetic
IYYIYANIC

SEQ ID NO: 266 - protein, 9 aa, Artificial
  Synthetic
IYYFYANIC

SEQ ID NO: 267 - protein, 9 aa, Artificial
  Synthetic
IYYLYGNIC

SEQ ID NO: 268 - protein, 9 aa, Artificial
  Synthetic
LYYIYANIC

SEQ ID NO: 269 - protein, 9 aa, Artificial
  Synthetic
LYYFYANIC

SEQ ID NO: 270 - protein, 9 aa, Artificial
  Synthetic
LYYLYGNIC

SEQ ID NO: 271 - protein, 9 aa, Artificial
  Synthetic
FHHLYANIC

SEQ ID NO: 272 - protein, 9 aa, Artificial
  Synthetic
FFHLYANIC

SEQ ID NO: 273 - protein, 9 aa, Artificial
  Synthetic
FYHIYANIC

SEQ ID NO: 274 - protein, 9 aa, Artificial
  Synthetic
FYHFYANIC

SEQ ID NO: 275 - protein, 9 aa, Artificial
  Synthetic
FYHLYGNIC

SEQ ID NO: 276 - protein, 9 aa, Artificial
  Synthetic
FYHLYASIC

SEQ ID NO: 277 - protein, 9 aa, Artificial
  Synthetic
FYYIYGNIC

SEQ ID NO: 278 - protein, 9 aa, Artificial
  Synthetic
FYYIYASIC

SEQ ID NO: 279 - protein, 9 aa, Artificial
  Synthetic
FYYIYAKIC

SEQ ID NO: 280 - protein, 9 aa, Artificial
  Synthetic
FYYFYASIC

SEQ ID NO: 281 - protein, 9 aa, Artificial
  Synthetic
FYYFYATIC

SEQ ID NO: 282 - protein, 9 aa, Artificial
  Synthetic
FYYFYAQIC

SEQ ID NO: 283 - protein, 9 aa, Artificial
  Synthetic
FYYFYAHIC

SEQ ID NO: 284 - protein, 9 aa, Artificial
  Synthetic
FYYFYARIC

SEQ ID NO: 285 - protein, 9 aa, Artificial
  Synthetic
FYYFYAKIC

SEQ ID NO: 286 - protein, 9 aa, Artificial
  Synthetic
FYYLYSRIC

SEQ ID NO: 287 - protein, 9 aa, Artificial
  Synthetic
FYYLYSKIC

SEQ ID NO: 288 - protein, 9 aa, Artificial
  Synthetic
YYLWANICF

SEQ ID NO: 289 - protein, 9 aa, Artificial
  Synthetic
YYLYAEICF

SEQ ID NO: 290 - protein, 9 aa, Artificial
  Synthetic
YFLYAQICF

SEQ ID NO: 291 - protein, 9 aa, Artificial
  Synthetic
YWVYANICF

SEQ ID NO: 292 - protein, 9 aa, Artificial
  Synthetic
YWLHANICF

SEQ ID NO: 293 - protein, 9 aa, Artificial
  Synthetic
YWLYADICF

SEQ ID NO: 294 - protein, 9 aa, Artificial
  Synthetic
YWLYAHICF

SEQ ID NO: 295 - protein, 9 aa, Artificial
  Synthetic
YWLYANVCF

SEQ ID NO: 296 - protein, 9 aa, Artificial
  Synthetic
YWLYANICY

SEQ ID NO: 297 - protein, 9 aa, Artificial
  Synthetic
YWLYANICW

SEQ ID NO: 298 - protein, 9 aa, Artificial
  Synthetic
YYVHANICF

SEQ ID NO: 299 - protein, 9 aa, Artificial
  Synthetic
YYVYADICF

SEQ ID NO: 300 - protein, 9 aa, Artificial
  Synthetic
YYVYAQICF

SEQ ID NO: 301 - protein, 9 aa, Artificial
  Synthetic
YYVYAHICF

SEQ ID NO: 302 - protein, 9 aa, Artificial
  Synthetic
YYVYANVCF

SEQ ID NO: 303 - protein, 9 aa, Artificial
  Synthetic
YYVYANICW

SEQ ID NO: 304 - protein, 9 aa, Artificial
  Synthetic
YYFHANICF

SEQ ID NO: 305 - protein, 9 aa, Artificial
  Synthetic
YYFYAQICF

SEQ ID NO: 306 - protein, 9 aa, Artificial
  Synthetic
YYLHADICF

SEQ ID NO: 307 - protein, 9 aa, Artificial
  Synthetic
YYLHAHICF

SEQ ID NO: 308 - protein, 9 aa, Artificial
  Synthetic
YYLHANVCF

SEQ ID NO: 309 - protein, 9 aa, Artificial
  Synthetic
YYLHANICY

SEQ ID NO: 310 - protein, 9 aa, Artificial
  Synthetic
YYLHANICW

SEQ ID NO: 311 - protein, 9 aa, Artificial
  Synthetic
YYLFAQICF

SEQ ID NO: 312 - protein, 9 aa, Artificial
  Synthetic
YYLYADVCF

SEQ ID NO: 313 - protein, 9 aa, Artificial
  Synthetic
YYLYADICY

SEQ ID NO: 314 - protein, 9 aa, Artificial
  Synthetic
YYLYADICW

SEQ ID NO: 315 - protein, 9 aa, Artificial
  Synthetic
YYLYAQVCF

SEQ ID NO: 316 - protein, 9 aa, Artificial
  Synthetic
YYLYAQFCF

SEQ ID NO: 317 - protein, 9 aa, Artificial
  Synthetic
YYLYAQICY

SEQ ID NO: 318 - protein, 9 aa, Artificial
  Synthetic
YYLYAHVCF

SEQ ID NO: 319 - protein, 9 aa, Artificial
  Synthetic
YYLYAHICY

SEQ ID NO: 320 - protein, 9 aa, Artificial
  Synthetic
YYLYAHICW

SEQ ID NO: 321 - protein, 9 aa, Artificial
  Synthetic
YYLYANVCY

SEQ ID NO: 322 - protein, 9 aa, Artificial
  Synthetic
YYLYANVCW

SEQ ID NO: 323 - protein, 9 aa, Artificial
  Synthetic
YHFYANVCF

SEQ ID NO: 324 - protein, 9 aa, Artificial
  Synthetic
YFFYANVCF

SEQ ID NO: 325 - protein, 9 aa, Artificial
  Synthetic
YYFFANVCF

SEQ ID NO: 326 - protein, 9 aa, Artificial
  Synthetic
YYFYASVCF

SEQ ID NO: 327 - protein, 9 aa, Artificial
  Synthetic
MTYVTKTSI

SEQ ID NO: 328 - protein, 9 aa, Artificial
  Synthetic
MAYVTKTSI

SEQ ID NO: 329 - protein, 9 aa, Artificial
  Synthetic
MMYVTKTSI

SEQ ID NO: 330 - protein, 9 aa, Artificial
  Synthetic
MIYVTKTSI

SEQ ID NO: 331 - protein, 9 aa, Artificial
  Synthetic
MLYVTKTSI

SEQ ID NO: 332 - protein, 9 aa, Artificial
  Synthetic
MVHVTKTSI

SEQ ID NO: 333 - protein, 9 aa, Artificial
  Synthetic
MVWVTKTSI

SEQ ID NO: 334 - protein, 9 aa, Artificial
  Synthetic
MVYTTKTSI

SEQ ID NO: 335 - protein, 9 aa, Artificial
  Synthetic
MVYVTETSI

SEQ ID NO: 336 - protein, 9 aa, Artificial
  Synthetic
MVYVTQTSI

SEQ ID NO: 337 - protein, 9 aa, Artificial
  Synthetic
MVYVTRTSI

SEQ ID NO: 338 - protein, 9 aa, Artificial
  Synthetic
MVYVTKTSL

SEQ ID NO: 339 - protein, 9 aa, Artificial
  Synthetic
MVYVTKTSV

SEQ ID NO: 340 - protein, 9 aa, Artificial
  Synthetic
FVYVTKSSI

SEQ ID NO: 341 - protein, 9 aa, Artificial
  Synthetic
FVYVTKTSM

SEQ ID NO: 342 - protein, 9 aa, Artificial
  Synthetic
FVYVTKTSF

SEQ ID NO: 343 - protein, 9 aa, Artificial
  Synthetic
YTTKTSIKI

SEQ ID NO: 344 - protein, 9 aa, Artificial
  Synthetic
YATKTSIKI

SEQ ID NO: 345 - protein, 9 aa, Artificial
  Synthetic
YMTKTSIKI

SEQ ID NO: 346 - protein, 9 aa, Artificial
  Synthetic
YITKTSIKI

SEQ ID NO: 347 - protein, 9 aa, Artificial
  Synthetic
YLTKTSIKI

SEQ ID NO: 348 - protein, 9 aa, Artificial
  Synthetic
YVTKTGIKI

SEQ ID NO: 349 - protein, 9 aa, Artificial
  Synthetic
YVTKTNIKI

SEQ ID NO: 350 - protein, 9 aa, Artificial
  Synthetic
YVTKTDIKI

SEQ ID NO: 351 - protein, 9 aa, Artificial
  Synthetic
YVTKTEIKI

SEQ ID NO: 352 - protein, 9 aa, Artificial
  Synthetic
YVTKTQIKI

SEQ ID NO: 353 - protein, 9 aa, Artificial
  Synthetic
YVTKTSVKI

SEQ ID NO: 354 - protein, 9 aa, Artificial
  Synthetic
YVTKTSIKV

SEQ ID NO: 355 - protein, 9 aa, Artificial
  Synthetic
YVTKTSIKF

SEQ ID NO: 356 - protein, 9 aa, Artificial
  Synthetic
YVTETKIKI

SEQ ID NO: 357 - protein, 9 aa, Artificial
  Synthetic
VSGFFKLRS

SEQ ID NO: 358 - protein, 9 aa, Artificial
  Synthetic
VGGYFKLRS

SEQ ID NO: 359 - protein, 9 aa, Artificial
  Synthetic
VGGFFELRS

SEQ ID NO: 360 - protein, 9 aa, Artificial
  Synthetic
VGGFFQLRS

SEQ ID NO: 361 - protein, 9 aa, Artificial
  Synthetic
VGGFFKVRS

SEQ ID NO: 362 - protein, 9 aa, Artificial
  Synthetic
VGGFFKLRT

SEQ ID NO: 363 - protein, 9 aa, Artificial
  Synthetic
VGGFFKLRG

SEQ ID NO: 364 - protein, 9 aa, Artificial
  Synthetic
VGGFFKLRN

SEQ ID NO: 365 - protein, 9 aa, Artificial
  Synthetic
VGGFFKLRD

SEQ ID NO: 366 - protein, 9 aa, Artificial
  Synthetic
VGGFFKLRE

SEQ ID NO: 367 - protein, 9 aa, Artificial
  Synthetic
VGGFFKLRK

SEQ ID NO: 368 - protein, 9 aa, Artificial
  Synthetic
VAGFFKIRS

SEQ ID NO: 369 - protein, 9 aa, Artificial
  Synthetic
VAGFFKLRA

SEQ ID NO: 370 - protein, 9 aa, Artificial
  Synthetic
VGAFFKIRS

SEQ ID NO: 371 - protein, 9 aa, Artificial
  Synthetic
VGAFFKLRA

SEQ ID NO: 372 - protein, 9 aa, Artificial
  Synthetic
VGGWFRLRS

SEQ ID NO: 373 - protein, 9 aa, Artificial
  Synthetic
VGGWFKIRS

SEQ ID NO: 374 - protein, 9 aa, Artificial
  Synthetic
VGGWFKLRA

SEQ ID NO: 375 - protein, 9 aa, Artificial
  Synthetic
VGGFFSIRS

SEQ ID NO: 376 - protein, 9 aa, Artificial
  Synthetic
VGGFFSFRS

SEQ ID NO: 377 - protein, 9 aa, Artificial
  Synthetic
VGGFFSLRQ

SEQ ID NO: 378 - protein, 9 aa, Artificial
  Synthetic
VGGFFNIRS

SEQ ID NO: 379 - protein, 9 aa, Artificial
  Synthetic
VGGFFNFRS

SEQ ID NO: 380 - protein, 9 aa, Artificial
  Synthetic
VGGFFKMRA

SEQ ID NO: 381 - protein, 9 aa, Artificial
  Synthetic
VGGFFKIRA

SEQ ID NO: 382 - protein, 9 aa, Artificial
  Synthetic
VGGFFKIRQ

SEQ ID NO: 383 - protein, 9 aa, Artificial
  Synthetic
VGGFFKFRA

SEQ ID NO: 384 - protein, 9 aa, Artificial
  Synthetic
IDIEVSNPS

SEQ ID NO: 385 - protein, 9 aa, Artificial
  Synthetic
ISLEVSNPS

SEQ ID NO: 386 - protein, 9 aa, Artificial
  Synthetic
ISVEVSNPS

SEQ ID NO: 387 - protein, 9 aa, Artificial
  Synthetic
ISFEVSNPS

SEQ ID NO: 388 - protein, 9 aa, Artificial
  Synthetic
ISISVSNPS

SEQ ID NO: 389 - protein, 9 aa, Artificial
  Synthetic
ISINVSNPS

SEQ ID NO: 390 - protein, 9 aa, Artificial
  Synthetic
ISIQVSNPS

SEQ ID NO: 391 - protein, 9 aa, Artificial
  Synthetic
ISIKVSNPS

SEQ ID NO: 392 - protein, 9 aa, Artificial
  Synthetic
ISIEVANPS

SEQ ID NO: 393 - protein, 9 aa, Artificial
  Synthetic
ISIEVGNPS

SEQ ID NO: 394 - protein, 9 aa, Artificial
  Synthetic
ISIEVDNPS

SEQ ID NO: 395 - protein, 9 aa, Artificial
  Synthetic
ISIEVENPS

SEQ ID NO: 396 - protein, 9 aa, Artificial
  Synthetic
ISIEVQNPS

SEQ ID NO: 397 - protein, 9 aa, Artificial
  Synthetic
ISIEVKNPS

SEQ ID NO: 398 - protein, 9 aa, Artificial
  Synthetic
ISIEVSSPS

SEQ ID NO: 399 - protein, 9 aa, Artificial
  Synthetic
ISIEVSTPS

SEQ ID NO: 400 - protein, 9 aa, Artificial
  Synthetic
ISIEVSGPS

SEQ ID NO: 401 - protein, 9 aa, Artificial
  Synthetic
ISIEVSDPS

SEQ ID NO: 402 - protein, 9 aa, Artificial
  Synthetic
ISIEVSQPS

SEQ ID NO: 403 - protein, 9 aa, Artificial
  Synthetic
ISIEVSHPS

SEQ ID NO: 404 - protein, 9 aa, Artificial
  Synthetic
ISIEVSRPS

SEQ ID NO: 405 - protein, 9 aa, Artificial
  Synthetic
ISIEVSKPS

SEQ ID NO: 406 - protein, 9 aa, Artificial
  Synthetic
ISIEVSNPT

SEQ ID NO: 407 - protein, 9 aa, Artificial
  Synthetic
ISIEVSNPA

SEQ ID NO: 408 - protein, 9 aa, Artificial
  Synthetic
ISIEVSNPG

SEQ ID NO: 409 - protein, 9 aa, Artificial
  Synthetic
ISIEVSNPN

SEQ ID NO: 410 - protein, 9 aa, Artificial
  Synthetic
ISIEVSNPD

SEQ ID NO: 411 - protein, 9 aa, Artificial
  Synthetic
ISIEVSNPE

SEQ ID NO: 412 - protein, 9 aa, Artificial
  Synthetic
ISIEVSNPK

SEQ ID NO: 413 - protein, 9 aa, Artificial
  Synthetic
LNRGRGLQA

SEQ ID NO: 414 - protein, 9 aa, Artificial
  Synthetic
LERGRGLQA

SEQ ID NO: 415 - protein, 9 aa, Artificial
  Synthetic
LQRGRGLQA

SEQ ID NO: 416 - protein, 9 aa, Artificial
  Synthetic
LHRGRGLQA

SEQ ID NO: 417 - protein, 9 aa, Artificial
  Synthetic
LKRGRGLQA

SEQ ID NO: 418 - protein, 9 aa, Artificial
  Synthetic
LREGRGLQA

SEQ ID NO: 419 - protein, 9 aa, Artificial
  Synthetic
LRQGRGLQA

SEQ ID NO: 420 - protein, 9 aa, Artificial
  Synthetic
LRHGRGLQA

SEQ ID NO: 421 - protein, 9 aa, Artificial
  Synthetic
LRKGRGLQA

SEQ ID NO: 422 - protein, 9 aa, Artificial
  Synthetic
LRRSRGLQA

SEQ ID NO: 423 - protein, 9 aa, Artificial
  Synthetic
LRRGRGFQA

SEQ ID NO: 424 - protein, 9 aa, Artificial
  Synthetic
LRRGRGLQS

SEQ ID NO: 425 - protein, 9 aa, Artificial
  Synthetic
LRRGRGLQG

SEQ ID NO: 426 - protein, 9 aa, Artificial
  Synthetic
LRNGRGMQA

SEQ ID NO: 427 - protein, 9 aa, Artificial
  Synthetic
LRNGRGIQA

SEQ ID NO: 428 - protein, 9 aa, Artificial
  Synthetic
LRNGRGVQA

SEQ ID NO: 429 - protein, 9 aa, Artificial
  Synthetic
LRRGRSIQA

SEQ ID NO: 430 - protein, 9 aa, Artificial
  Synthetic
LRRGRAMQA

SEQ ID NO: 431 - protein, 9 aa, Artificial
  Synthetic
LRRGRGMQT

SEQ ID NO: 432 - protein, 9 aa, Artificial
  Synthetic
LRRGRGIQT

SEQ ID NO: 433 - protein, 9 aa, Artificial
  Synthetic
LRRGRGVQT

SEQ ID NO: 434 - protein, 9 aa, Artificial
  Synthetic
LLHSQVLFQ

SEQ ID NO: 435 - protein, 9 aa, Artificial
  Synthetic
LLWSQVLFQ

SEQ ID NO: 436 - protein, 9 aa, Artificial
  Synthetic
LLYGQVLFQ

SEQ ID NO: 437 - protein, 9 aa, Artificial
  Synthetic
LLYSQALFQ

SEQ ID NO: 438 - protein, 9 aa, Artificial
  Synthetic
LLYSQMLFQ

SEQ ID NO: 439 - protein, 9 aa, Artificial
  Synthetic
LLYSQILFQ

SEQ ID NO: 440 - protein, 9 aa, Artificial
  Synthetic
LLYSQLLFQ

SEQ ID NO: 441 - protein, 9 aa, Artificial
  Synthetic
LLYSQVIFQ

SEQ ID NO: 442 - protein, 9 aa, Artificial
  Synthetic
LLYSQVVFQ

SEQ ID NO: 443 - protein, 9 aa, Artificial
  Synthetic
LLYSQVFFQ

SEQ ID NO: 444 - protein, 9 aa, Artificial
  Synthetic
LLYSQVLFN

SEQ ID NO: 445 - protein, 9 aa, Artificial
  Synthetic
LLYSQVLFD

SEQ ID NO: 446 - protein, 9 aa, Artificial
  Synthetic
LLYSQVLFE

SEQ ID NO: 447 - protein, 9 aa, Artificial
  Synthetic
LLYSQVLFH

SEQ ID NO: 448 - protein, 9 aa, Artificial
  Synthetic
LLYSQVLFR

SEQ ID NO: 449 - protein, 9 aa, Artificial
  Synthetic
LLYSQVLFK

SEQ ID NO: 450 - protein, 9 aa, Artificial
  Synthetic
LLYTQVLFM

SEQ ID NO: 451 - protein, 9 aa, Artificial
  Synthetic
FFQDVTFTM

SEQ ID NO: 452 - protein, 9 aa, Artificial
  Synthetic
LWQDVTFTM

SEQ ID NO: 453 - protein, 9 aa, Artificial
  Synthetic
LFDDVTFTM

SEQ ID NO: 454 - protein, 9 aa, Artificial
  Synthetic
LFEDVTFTM

SEQ ID NO: 455 - protein, 9 aa, Artificial
  Synthetic
LFQSVTFTM

SEQ ID NO: 456 - protein, 9 aa, Artificial
  Synthetic
LFQNVTFTM

SEQ ID NO: 457 - protein, 9 aa, Artificial
  Synthetic
LFQEVTFTM

SEQ ID NO: 458 - protein, 9 aa, Artificial
  Synthetic
LFQQVTFTM

SEQ ID NO: 459 - protein, 9 aa, Artificial
  Synthetic
LFQDVSFTM

SEQ ID NO: 460 - protein, 9 aa, Artificial
  Synthetic
LFQDVAFTM

SEQ ID NO: 461 - protein, 9 aa, Artificial
  Synthetic
LFQDVNFTM

SEQ ID NO: 462 - protein, 9 aa, Artificial
  Synthetic
LFQDVTWTM

SEQ ID NO: 463 - protein, 9 aa, Artificial
  Synthetic
LFQDVTFTQ

SEQ ID NO: 464 - protein, 9 aa, Artificial
  Synthetic
LFQDVTFTL

SEQ ID NO: 465 - protein, 9 aa, Artificial
  Synthetic
LFQDVTFTV

SEQ ID NO: 466 - protein, 9 aa, Artificial
  Synthetic
LFQDVTYTI

SEQ ID NO: 467 - protein, 9 aa, Artificial
  Synthetic
FNCIRGMPS

SEQ ID NO: 468 - protein, 9 aa, Artificial
  Synthetic
FNCIRDMPS

SEQ ID NO: 469 - protein, 9 aa, Artificial
  Synthetic
FNCIREMPS

SEQ ID NO: 470 - protein, 9 aa, Artificial
  Synthetic
FNCIRSMPT

SEQ ID NO: 471 - protein, 9 aa, Artificial
  Synthetic
FNCIRSMPG

SEQ ID NO: 472 - protein, 9 aa, Artificial
  Synthetic
FNCIRSMPK

SEQ ID NO: 473 - protein, 9 aa, Artificial
  Synthetic
FECVRSMPS

SEQ ID NO: 474 - protein, 9 aa, Artificial
  Synthetic
FECIRAMPS

SEQ ID NO: 475 - protein, 9 aa, Artificial
  Synthetic
FECIRGMPS

SEQ ID NO: 476 - protein, 9 aa, Artificial
  Synthetic
FECIRDMPS

SEQ ID NO: 477 - protein, 9 aa, Artificial
  Synthetic
FECIREMPS

SEQ ID NO: 478 - protein, 9 aa, Artificial
  Synthetic
FECIRQMPS

SEQ ID NO: 479 - protein, 9 aa, Artificial
  Synthetic
FECIRSQPS

SEQ ID NO: 480 - protein, 9 aa, Artificial
  Synthetic
FECIRSIPS

SEQ ID NO: 481 - protein, 9 aa, Artificial
  Synthetic
FECIRSLPS

SEQ ID NO: 482 - protein, 9 aa, Artificial
  Synthetic
FECIRSVPS

SEQ ID NO: 483 - protein, 9 aa, Artificial
  Synthetic
FECIRSFPS

SEQ ID NO: 484 - protein, 9 aa, Artificial
  Synthetic
FECIRSMPT

SEQ ID NO: 485 - protein, 9 aa, Artificial
  Synthetic
FECIRSMPA

SEQ ID NO: 486 - protein, 9 aa, Artificial
  Synthetic
FECIRSMPG

SEQ ID NO: 487 - protein, 9 aa, Artificial
  Synthetic
FECIRSMPN

SEQ ID NO: 488 - protein, 9 aa, Artificial
  Synthetic
FECIRSMPD

SEQ ID NO: 489 - protein, 9 aa, Artificial
  Synthetic
FECIRSMPK

SEQ ID NO: 490 - protein, 9 aa, Artificial
  Synthetic
FQCIRDMPS

SEQ ID NO: 491 - protein, 9 aa, Artificial
  Synthetic
FQCIREMPS

SEQ ID NO: 492 - protein, 9 aa, Artificial
  Synthetic
FQCIRSMPT

SEQ ID NO: 493 - protein, 9 aa, Artificial
  Synthetic
FHCIRGMPS

SEQ ID NO: 494 - protein, 9 aa, Artificial
  Synthetic
FHCIRDMPS

SEQ ID NO: 495 - protein, 9 aa, Artificial
  Synthetic
FHCIREMPS

SEQ ID NO: 496 - protein, 9 aa, Artificial
  Synthetic
FHCIRSMPT

SEQ ID NO: 497 - protein, 9 aa, Artificial
  Synthetic
FHCIRSMPG

SEQ ID NO: 498 - protein, 9 aa, Artificial
  Synthetic
FHCIRSMPK

SEQ ID NO: 499 - protein, 9 aa, Artificial
  Synthetic
FKCIRGMPS

SEQ ID NO: 500 - protein, 9 aa, Artificial
  Synthetic
FKCIRDMPS

SEQ ID NO: 501 - protein, 9 aa, Artificial
  Synthetic
FKCIREMPS

SEQ ID NO: 502 - protein, 9 aa, Artificial
  Synthetic
FKCIRSMPT

SEQ ID NO: 503 - protein, 9 aa, Artificial
  Synthetic
FRCVRDMPS

SEQ ID NO: 504 - protein, 9 aa, Artificial
  Synthetic
FRCVREMPS

SEQ ID NO: 505 - protein, 9 aa, Artificial
  Synthetic
FRCIRGMPG

SEQ ID NO: 506 - protein, 9 aa, Artificial
  Synthetic
FRCIRGMPN

SEQ ID NO: 507 - protein, 9 aa, Artificial
  Synthetic
FRCIRGMPD

SEQ ID NO: 508 - protein, 9 aa, Artificial
  Synthetic
FRCIRGMPE

SEQ ID NO: 509 - protein, 9 aa, Artificial
  Synthetic
FRCIRDQPS

SEQ ID NO: 510 - protein, 9 aa, Artificial
  Synthetic
FRCIRDIPS

SEQ ID NO: 511 - protein, 9 aa, Artificial
  Synthetic
FRCIRDLPS

SEQ ID NO: 512 - protein, 9 aa, Artificial
  Synthetic
FRCIRDVPS

SEQ ID NO: 513 - protein, 9 aa, Artificial
  Synthetic
FRCIRDFPS

SEQ ID NO: 514 - protein, 9 aa, Artificial
  Synthetic
FRCIRDMPT

SEQ ID NO: 515 - protein, 9 aa, Artificial
  Synthetic
FRCIRDMPA

SEQ ID NO: 516 - protein, 9 aa, Artificial
  Synthetic
FRCIRDMPG

SEQ ID NO: 517 - protein, 9 aa, Artificial
  Synthetic
FRCIRDMPN

SEQ ID NO: 518 - protein, 9 aa, Artificial
  Synthetic
FRCIRDMPD

SEQ ID NO: 519 - protein, 9 aa, Artificial
  Synthetic
FRCIRDMPK

SEQ ID NO: 520 - protein, 9 aa, Artificial
  Synthetic
FRCIREQPS

SEQ ID NO: 521 - protein, 9 aa, Artificial
  Synthetic
FRCIREIPS

SEQ ID NO: 522 - protein, 9 aa, Artificial
  Synthetic
FRCIREVPS

SEQ ID NO: 523 - protein, 9 aa, Artificial
  Synthetic
FRCIREFPS

SEQ ID NO: 524 - protein, 9 aa, Artificial
  Synthetic
FRCIREMPT

SEQ ID NO: 525 - protein, 9 aa, Artificial
  Synthetic
FRCIREMPA

SEQ ID NO: 526 - protein, 9 aa, Artificial
  Synthetic
FRCIREMPG

SEQ ID NO: 527 - protein, 9 aa, Artificial
  Synthetic
FRCIREMPN

SEQ ID NO: 528 - protein, 9 aa, Artificial
  Synthetic
FRCIREMPD

SEQ ID NO: 529 - protein, 9 aa, Artificial
  Synthetic
FRCIREMPE

SEQ ID NO: 530 - protein, 9 aa, Artificial
  Synthetic
FRCIREMPK

SEQ ID NO: 531 - protein, 9 aa, Artificial
  Synthetic
FRCIRQQPS

SEQ ID NO: 532 - protein, 9 aa, Artificial
  Synthetic
FRCIRQVPS

SEQ ID NO: 533 - protein, 9 aa, Artificial
  Synthetic
FRCIRQFPS

SEQ ID NO: 534 - protein, 9 aa, Artificial
  Synthetic
FRCIRQMPT

SEQ ID NO: 535 - protein, 9 aa, Artificial
  Synthetic
FRCIRQMPG

SEQ ID NO: 536 - protein, 9 aa, Artificial
  Synthetic
FRCIRQMPN

SEQ ID NO: 537 - protein, 9 aa, Artificial
  Synthetic
FRCIRQMPD

SEQ ID NO: 538 - protein, 9 aa, Artificial
  Synthetic
FRCIRQMPE

SEQ ID NO: 539 - protein, 9 aa, Artificial
  Synthetic
FNCVRAMPS

SEQ ID NO: 540 - protein, 9 aa, Artificial
  Synthetic
FNCVRSLPS

SEQ ID NO: 541 - protein, 9 aa, Artificial
  Synthetic
FNCVRSMPA

SEQ ID NO: 542 - protein, 9 aa, Artificial
  Synthetic
FNCVRSMPN

SEQ ID NO: 543 - protein, 9 aa, Artificial
  Synthetic
FQCVRGMPS

SEQ ID NO: 544 - protein, 9 aa, Artificial
  Synthetic
FQCVRQMPS

SEQ ID NO: 545 - protein, 9 aa, Artificial
  Synthetic
FQCVRSQPS

SEQ ID NO: 546 - protein, 9 aa, Artificial
  Synthetic
FQCVRSVPS

SEQ ID NO: 547 - protein, 9 aa, Artificial
  Synthetic
FQCVRSFPS

SEQ ID NO: 548 - protein, 9 aa, Artificial
  Synthetic
FQCVRSMPG

SEQ ID NO: 549 - protein, 9 aa, Artificial
  Synthetic
FQCVRSMPN

SEQ ID NO: 550 - protein, 9 aa, Artificial
  Synthetic
FQCVRSMPK

SEQ ID NO: 551 - protein, 9 aa, Artificial
  Synthetic
FHCVRAMPS

SEQ ID NO: 552 - protein, 9 aa, Artificial
  Synthetic
FHCVRSLPS

SEQ ID NO: 553 - protein, 9 aa, Artificial
  Synthetic
FHCVRSMPA

SEQ ID NO: 554 - protein, 9 aa, Artificial
  Synthetic
FHCVRSMPN

SEQ ID NO: 555 - protein, 9 aa, Artificial
  Synthetic
FKCVRQMPS

SEQ ID NO: 556 - protein, 9 aa, Artificial
  Synthetic
FKCVRSQPS

SEQ ID NO: 557 - protein, 9 aa, Artificial
  Synthetic
FKCVRSIPS

SEQ ID NO: 558 - protein, 9 aa, Artificial
  Synthetic
FKCVRSVPS

SEQ ID NO: 559 - protein, 9 aa, Artificial
  Synthetic
FKCVRSFPS

SEQ ID NO: 560 - protein, 9 aa, Artificial
  Synthetic
FKCVRSMPA

SEQ ID NO: 561 - protein, 9 aa, Artificial
  Synthetic
FKCVRSMPG

SEQ ID NO: 562 - protein, 9 aa, Artificial
  Synthetic
FKCVRSMPN

SEQ ID NO: 563 - protein, 9 aa, Artificial
  Synthetic
FKCVRSMPK

SEQ ID NO: 564 - protein, 9 aa, Artificial
  Synthetic
FKCIRAMPA

SEQ ID NO: 565 - protein, 9 aa, Artificial
  Synthetic
FKCIRAMPN

SEQ ID NO: 566 - protein, 9 aa, Artificial
  Synthetic
FRCVRAQPS

SEQ ID NO: 567 - protein, 9 aa, Artificial
  Synthetic
FRCVRAMPT

SEQ ID NO: 568 - protein, 9 aa, Artificial
  Synthetic
FRCVRAMPG

SEQ ID NO: 569 - protein, 9 aa, Artificial
  Synthetic
FRCVRAMPN

SEQ ID NO: 570 - protein, 9 aa, Artificial
  Synthetic
FRCVRGQPS

SEQ ID NO: 571 - protein, 9 aa, Artificial
  Synthetic
FRCVRGFPS

SEQ ID NO: 572 - protein, 9 aa, Artificial
  Synthetic
FRCVRGMPT

SEQ ID NO: 573 - protein, 9 aa, Artificial
  Synthetic
FRCVRGMPA

SEQ ID NO: 574 - protein, 9 aa, Artificial
  Synthetic
FRCVRGMPK

SEQ ID NO: 575 - protein, 9 aa, Artificial
  Synthetic
FRCVRQMPK

SEQ ID NO: 576 - protein, 9 aa, Artificial
  Synthetic
FRCVRSQPT

SEQ ID NO: 577 - protein, 9 aa, Artificial
  Synthetic
FRCVRSQPG

SEQ ID NO: 578 - protein, 9 aa, Artificial
  Synthetic
FRCVRSQPK

SEQ ID NO: 579 - protein, 9 aa, Artificial
  Synthetic
FRCVRSIPT

SEQ ID NO: 580 - protein, 9 aa, Artificial
  Synthetic
FRCVRSIPG

SEQ ID NO: 581 - protein, 9 aa, Artificial
  Synthetic
FRCVRSVPT

SEQ ID NO: 582 - protein, 9 aa, Artificial
  Synthetic
FRCVRSVPG

SEQ ID NO: 583 - protein, 9 aa, Artificial
  Synthetic
FRCVRSVPK

SEQ ID NO: 584 - protein, 9 aa, Artificial
  Synthetic
FRCVRSFPT

SEQ ID NO: 585 - protein, 9 aa, Artificial
  Synthetic
FRCVRSFPG

SEQ ID NO: 586 - protein, 9 aa, Artificial
  Synthetic
FRCVRSFPK

SEQ ID NO: 587 - protein, 9 aa, Artificial
  Synthetic
LEAANTHSS

SEQ ID NO: 588 - protein, 9 aa, Artificial
  Synthetic
LRAANAHSS

SEQ ID NO: 589 - protein, 9 aa, Artificial
  Synthetic
LRAANTHST

SEQ ID NO: 590 - protein, 9 aa, Artificial
  Synthetic
LRAANTHSG

SEQ ID NO: 591 - protein, 9 aa, Artificial
  Synthetic
LRAANTHSN

SEQ ID NO: 592 - protein, 9 aa, Artificial
  Synthetic
LRAANTHSD

SEQ ID NO: 593 - protein, 9 aa, Artificial
  Synthetic
LRAANTHSE

SEQ ID NO: 594 - protein, 9 aa, Artificial
  Synthetic
FRAGNTHSS

SEQ ID NO: 595 - protein, 9 aa, Artificial
  Synthetic
LNASNTHSS

SEQ ID NO: 596 - protein, 9 aa, Artificial
  Synthetic
LNAANTHSA

SEQ ID NO: 597 - protein, 9 aa, Artificial
  Synthetic
LQASNTHSS

SEQ ID NO: 598 - protein, 9 aa, Artificial
  Synthetic
LQATNTHSS

SEQ ID NO: 599 - protein, 9 aa, Artificial
  Synthetic
LQAGNTHSS

SEQ ID NO: 600 - protein, 9 aa, Artificial
  Synthetic
LQAANSHSS

SEQ ID NO: 601 - protein, 9 aa, Artificial
  Synthetic
LQAANTHSA

SEQ ID NO: 602 - protein, 9 aa, Artificial
  Synthetic
LQAANTHSK

SEQ ID NO: 603 - protein, 9 aa, Artificial
  Synthetic
LHASNTHSS

SEQ ID NO: 604 - protein, 9 aa, Artificial
  Synthetic
LHAANTHSA

SEQ ID NO: 605 - protein, 9 aa, Artificial
  Synthetic
LKASNTHSS

SEQ ID NO: 606 - protein, 9 aa, Artificial
  Synthetic
LKATNTHSS

SEQ ID NO: 607 - protein, 9 aa, Artificial
  Synthetic
LKAGNTHSS

SEQ ID NO: 608 - protein, 9 aa, Artificial
  Synthetic
LKAANSHSS

SEQ ID NO: 609 - protein, 9 aa, Artificial
  Synthetic
LKAANNHSS

SEQ ID NO: 610 - protein, 9 aa, Artificial
  Synthetic
LKAANVHSS

SEQ ID NO: 611 - protein, 9 aa, Artificial
  Synthetic
LKAANTHSA

SEQ ID NO: 612 - protein, 9 aa, Artificial
  Synthetic
LKAANTHSK

SEQ ID NO: 613 - protein, 9 aa, Artificial
  Synthetic
LRATNTHSK

SEQ ID NO: 614 - protein, 9 aa, Artificial
  Synthetic
LRAGNSHSS

SEQ ID NO: 615 - protein, 9 aa, Artificial
  Synthetic
LRAGNTHSK

SEQ ID NO: 616 - protein, 9 aa, Artificial
  Synthetic
LRAANSHSA

SEQ ID NO: 617 - protein, 9 aa, Artificial
  Synthetic
LRAANSHSK

SEQ ID NO: 618 - protein, 9 aa, Artificial
  Synthetic
LTIHEKGFY

SEQ ID NO: 619 - protein, 9 aa, Artificial
  Synthetic
LAIHEKGFY

SEQ ID NO: 620 - protein, 9 aa, Artificial
  Synthetic
LMIHEKGFY

SEQ ID NO: 621 - protein, 9 aa, Artificial
  Synthetic
LIIHEKGFY

SEQ ID NO: 622 - protein, 9 aa, Artificial
  Synthetic
LLIHEKGFY

SEQ ID NO: 623 - protein, 9 aa, Artificial
  Synthetic
LVVHEKGFY

SEQ ID NO: 624 - protein, 9 aa, Artificial
  Synthetic
LVIEEKGFY

SEQ ID NO: 625 - protein, 9 aa, Artificial
  Synthetic
LVIHESGFY

SEQ ID NO: 626 - protein, 9 aa, Artificial
  Synthetic
LVIHENGFY

SEQ ID NO: 627 - protein, 9 aa, Artificial
  Synthetic
LVIHEEGFY

SEQ ID NO: 628 - protein, 9 aa, Artificial
  Synthetic
LVIHEQGFY

SEQ ID NO: 629 - protein, 9 aa, Artificial
  Synthetic
LVLHEKGFW

SEQ ID NO: 630 - protein, 9 aa, Artificial
  Synthetic
LVFHERGFY

SEQ ID NO: 631 - protein, 9 aa, Artificial
  Synthetic
LVFHEKGFW

SEQ ID NO: 632 - protein, 9 aa, Artificial
  Synthetic
LVIHERGFW

SEQ ID NO: 633 - protein, 9 aa, Artificial
  Synthetic
VSYIYKYTS

SEQ ID NO: 634 - protein, 9 aa, Artificial
  Synthetic
VDYIYKYTS

SEQ ID NO: 635 - protein, 9 aa, Artificial
  Synthetic
VEYIYKYTS

SEQ ID NO: 636 - protein, 9 aa, Artificial
  Synthetic
VQHIYKYTS

SEQ ID NO: 637 - protein, 9 aa, Artificial
  Synthetic
VQWIYKYTS

SEQ ID NO: 638 - protein, 9 aa, Artificial
  Synthetic
VQYIYEYTS

SEQ ID NO: 639 - protein, 9 aa, Artificial
  Synthetic
VQYIYQYTS

SEQ ID NO: 640 - protein, 9 aa, Artificial
  Synthetic
VQYIYKYTT

SEQ ID NO: 641 - protein, 9 aa, Artificial
  Synthetic
VQYIYKYTA

SEQ ID NO: 642 - protein, 9 aa, Artificial
  Synthetic
VQYIYKYTG

SEQ ID NO: 643 - protein, 9 aa, Artificial
  Synthetic
VQYIYKYTN

SEQ ID NO: 644 - protein, 9 aa, Artificial
  Synthetic
VQYIYKYTD

SEQ ID NO: 645 - protein, 9 aa, Artificial
  Synthetic
VQYIYKYTE

SEQ ID NO: 646 - protein, 9 aa, Artificial
  Synthetic
VQYIYKYTK

SEQ ID NO: 647 - protein, 9 aa, Artificial
  Synthetic
VNYVYKYTS

SEQ ID NO: 648 - protein, 9 aa, Artificial
  Synthetic
VNYIYRYTS

SEQ ID NO: 649 - protein, 9 aa, Artificial
  Synthetic
VHYVYKYTS

SEQ ID NO: 650 - protein, 9 aa, Artificial
  Synthetic
VHYIYRYTS

SEQ ID NO: 651 - protein, 9 aa, Artificial
  Synthetic
VQYVYRYTS

SEQ ID NO: 652 - protein, 9 aa, Artificial
  Synthetic
VQYVYKYTQ

SEQ ID NO: 653 - protein, 9 aa, Artificial
  Synthetic
LLMESARNS

SEQ ID NO: 654 - protein, 9 aa, Artificial
  Synthetic
LLMKSAENS

SEQ ID NO: 655 - protein, 9 aa, Artificial
  Synthetic
LLMKSARNT

SEQ ID NO: 656 - protein, 9 aa, Artificial
  Synthetic
LLMKSARNN

SEQ ID NO: 657 - protein, 9 aa, Artificial
  Synthetic
LFQKSARNS

SEQ ID NO: 658 - protein, 9 aa, Artificial
  Synthetic
LFMKSARND

SEQ ID NO: 659 - protein, 9 aa, Artificial
  Synthetic
LLQQSARNS

SEQ ID NO: 660 - protein, 9 aa, Artificial
  Synthetic
LLQKSGRNS

SEQ ID NO: 661 - protein, 9 aa, Artificial
  Synthetic
LLQKSAQNS

SEQ ID NO: 662 - protein, 9 aa, Artificial
  Synthetic
LLQKSAKNS

SEQ ID NO: 663 - protein, 9 aa, Artificial
  Synthetic
LLQKSARNA

SEQ ID NO: 664 - protein, 9 aa, Artificial
  Synthetic
LLQKSARNG

SEQ ID NO: 665 - protein, 9 aa, Artificial
  Synthetic
LLQKSARND

SEQ ID NO: 666 - protein, 9 aa, Artificial
  Synthetic
LLQKSARNE

SEQ ID NO: 667 - protein, 9 aa, Artificial
  Synthetic
LLQKSARNQ

SEQ ID NO: 668 - protein, 9 aa, Artificial
  Synthetic
LLQKSARNK

SEQ ID NO: 669 - protein, 9 aa, Artificial
  Synthetic
LLLSSARNS

SEQ ID NO: 670 - protein, 9 aa, Artificial
  Synthetic
LLLKSANNS

SEQ ID NO: 671 - protein, 9 aa, Artificial
  Synthetic
LLLKSAKNS

SEQ ID NO: 672 - protein, 9 aa, Artificial
  Synthetic
LLLKSARND

SEQ ID NO: 673 - protein, 9 aa, Artificial
  Synthetic
LLVKSGRNS

SEQ ID NO: 674 - protein, 9 aa, Artificial
  Synthetic
LLVKSAQNS

SEQ ID NO: 675 - protein, 9 aa, Artificial
  Synthetic
LLVKSAKNS

SEQ ID NO: 676 - protein, 9 aa, Artificial
  Synthetic
LLVKSARNA

SEQ ID NO: 677 - protein, 9 aa, Artificial
  Synthetic
LLVKSARNG

SEQ ID NO: 678 - protein, 9 aa, Artificial
  Synthetic
LLVKSARND

SEQ ID NO: 679 - protein, 9 aa, Artificial
  Synthetic
LLVKSARNE

SEQ ID NO: 680 - protein, 9 aa, Artificial
  Synthetic
LLVKSARNK

SEQ ID NO: 681 - protein, 9 aa, Artificial
  Synthetic
LLFKSAQNS

SEQ ID NO: 682 - protein, 9 aa, Artificial
  Synthetic
LLFKSAKNS

SEQ ID NO: 683 - protein, 9 aa, Artificial
  Synthetic
LLFKSARNG

SEQ ID NO: 684 - protein, 9 aa, Artificial
  Synthetic
LLFKSARND

SEQ ID NO: 685 - protein, 9 aa, Artificial
  Synthetic
LLFKSARNE

SEQ ID NO: 686 - protein, 9 aa, Artificial
  Synthetic
LLFKSARNK

SEQ ID NO: 687 - protein, 9 aa, Artificial
  Synthetic
LLMSSAKNS

SEQ ID NO: 688 - protein, 9 aa, Artificial
  Synthetic
LLMSSARNA

SEQ ID NO: 689 - protein, 9 aa, Artificial
  Synthetic
LLMNSAKNS

SEQ ID NO: 690 - protein, 9 aa, Artificial
  Synthetic
LLMQSAQNS

SEQ ID NO: 691 - protein, 9 aa, Artificial
  Synthetic
LLMQSARND

SEQ ID NO: 692 - protein, 9 aa, Artificial
  Synthetic
LLMQSARNE

SEQ ID NO: 693 - protein, 9 aa, Artificial
  Synthetic
LLMKSSRND

SEQ ID NO: 694 - protein, 9 aa, Artificial
  Synthetic
LLMKSGQNS

SEQ ID NO: 695 - protein, 9 aa, Artificial
  Synthetic
LLMKSGKNS

SEQ ID NO: 696 - protein, 9 aa, Artificial
  Synthetic
LLMKSGRNG

SEQ ID NO: 697 - protein, 9 aa, Artificial
  Synthetic
LLMKSGRND

SEQ ID NO: 698 - protein, 9 aa, Artificial
  Synthetic
LLMKSGRNE

SEQ ID NO: 699 - protein, 9 aa, Artificial
  Synthetic
LLMKSGRNK

SEQ ID NO: 700 - protein, 9 aa, Artificial
  Synthetic
LLMKSANNA

SEQ ID NO: 701 - protein, 9 aa, Artificial
  Synthetic
LLMKSAQNA

SEQ ID NO: 702 - protein, 9 aa, Artificial
  Synthetic
LLMKSAQNG

SEQ ID NO: 703 - protein, 9 aa, Artificial
  Synthetic
LLMKSAQND

SEQ ID NO: 704 - protein, 9 aa, Artificial
  Synthetic
LLMKSAQNE

SEQ ID NO: 705 - protein, 9 aa, Artificial
  Synthetic
LLMKSAQNK

SEQ ID NO: 706 - protein, 9 aa, Artificial
  Synthetic
LLMKSAHNA

SEQ ID NO: 707 - protein, 9 aa, Artificial
  Synthetic
LLMKSAKNA

SEQ ID NO: 708 - protein, 9 aa, Artificial
  Synthetic
LLMKSAKNG

SEQ ID NO: 709 - protein, 9 aa, Artificial
  Synthetic
LLMKSAKND

SEQ ID NO: 710 - protein, 9 aa, Artificial
  Synthetic
LLMKSAKNK

SEQ ID NO: 711 - protein, 9 aa, Artificial
  Synthetic
IWVSVTNEH

SEQ ID NO: 712 - protein, 9 aa, Artificial
  Synthetic
IFTSVTNEH

SEQ ID NO: 713 - protein, 9 aa, Artificial
  Synthetic
IFASVTNEH

SEQ ID NO: 714 - protein, 9 aa, Artificial
  Synthetic
IFVTVTNEH

SEQ ID NO: 715 - protein, 9 aa, Artificial
  Synthetic
IFVGVTNEH

SEQ ID NO: 716 - protein, 9 aa, Artificial
  Synthetic
IFVSVSNEH

SEQ ID NO: 717 - protein, 9 aa, Artificial
  Synthetic
IFVSVANEH

SEQ ID NO: 718 - protein, 9 aa, Artificial
  Synthetic
IFVSVNNEH

SEQ ID NO: 719 - protein, 9 aa, Artificial
  Synthetic
IFVSVVNEH

SEQ ID NO: 720 - protein, 9 aa, Artificial
  Synthetic
IFVSVTSEH

SEQ ID NO: 721 - protein, 9 aa, Artificial
  Synthetic
IFVSVTTEH

SEQ ID NO: 722 - protein, 9 aa, Artificial
  Synthetic
IFVSVTGEH

SEQ ID NO: 723 - protein, 9 aa, Artificial
  Synthetic
IFVSVTDEH

SEQ ID NO: 724 - protein, 9 aa, Artificial
  Synthetic
IFVSVTEEH

SEQ ID NO: 725 - protein, 9 aa, Artificial
  Synthetic
IFVSVTQEH

SEQ ID NO: 726 - protein, 9 aa, Artificial
  Synthetic
IFVSVTHEH

SEQ ID NO: 727 - protein, 9 aa, Artificial
  Synthetic
IFVSVTKEH

SEQ ID NO: 728 - protein, 9 aa, Artificial
  Synthetic
IFVSVTNEN

SEQ ID NO: 729 - protein, 9 aa, Artificial
  Synthetic
IFVSVTNEE

SEQ ID NO: 730 - protein, 9 aa, Artificial
  Synthetic
IFVSVTNER

SEQ ID NO: 731 - protein, 9 aa, Artificial
  Synthetic
IFVSVTNEY

SEQ ID NO: 732 - protein, 9 aa, Artificial
  Synthetic
MVSVTNEHL

SEQ ID NO: 733 - protein, 9 aa, Artificial
  Synthetic
IVSVTNEHL

SEQ ID NO: 734 - protein, 9 aa, Artificial
  Synthetic
LVSVTNEHL

SEQ ID NO: 735 - protein, 9 aa, Artificial
  Synthetic
FTSVTNEHL

SEQ ID NO: 736 - protein, 9 aa, Artificial
  Synthetic
FASVTNEHL

SEQ ID NO: 737 - protein, 9 aa, Artificial
  Synthetic
FMSVTNEHL

SEQ ID NO: 738 - protein, 9 aa, Artificial
  Synthetic
FISVTNEHL

SEQ ID NO: 739 - protein, 9 aa, Artificial
  Synthetic
FLSVTNEHL

SEQ ID NO: 740 - protein, 9 aa, Artificial
  Synthetic
FVDVTNEHL

SEQ ID NO: 741 - protein, 9 aa, Artificial
  Synthetic
FVEVTNEHL

SEQ ID NO: 742 - protein, 9 aa, Artificial
  Synthetic
FVSATNEHL

SEQ ID NO: 743 - protein, 9 aa, Artificial
  Synthetic
FVSVTDEHL

SEQ ID NO: 744 - protein, 9 aa, Artificial
  Synthetic
FVSVTEEHL

SEQ ID NO: 745 - protein, 9 aa, Artificial
  Synthetic
FVSVTQEHL

SEQ ID NO: 746 - protein, 9 aa, Artificial
  Synthetic
FVSVTREHL

SEQ ID NO: 747 - protein, 9 aa, Artificial
  Synthetic
FVSVTKEHL

SEQ ID NO: 748 - protein, 9 aa, Artificial
  Synthetic
FVSVTNDHL

SEQ ID NO: 749 - protein, 9 aa, Artificial
  Synthetic
FVSVTNKHL

SEQ ID NO: 750 - protein, 9 aa, Artificial
  Synthetic
FVSVTNEHV

SEQ ID NO: 751 - protein, 9 aa, Artificial
  Synthetic
FVSVTNEHF

SEQ ID NO: 752 - protein, 9 aa, Artificial
  Synthetic
FVTLTNEHL

SEQ ID NO: 753 - protein, 9 aa, Artificial
  Synthetic
FVTVTHEHL

SEQ ID NO: 754 - protein, 9 aa, Artificial
  Synthetic
FVTVTNRHL

SEQ ID NO: 755 - protein, 9 aa, Artificial
  Synthetic
FVTVTNEHM

SEQ ID NO: 756 - protein, 9 aa, Artificial
  Synthetic
FVALTNEHL

SEQ ID NO: 757 - protein, 9 aa, Artificial
  Synthetic
FVAVTHEHL

SEQ ID NO: 758 - protein, 9 aa, Artificial
  Synthetic
FVAVTNRHL

SEQ ID NO: 759 - protein, 9 aa, Artificial
  Synthetic
FVAVTNEHM

SEQ ID NO: 760 - protein, 9 aa, Artificial
  Synthetic
FVQLTNEHL

SEQ ID NO: 761 - protein, 9 aa, Artificial
  Synthetic
FVQVTHEHL

SEQ ID NO: 762 - protein, 9 aa, Artificial
  Synthetic
FVQVTNRHL

SEQ ID NO: 763 - protein, 9 aa, Artificial
  Synthetic
FVQVTNEHM

SEQ ID NO: 764 - protein, 9 aa, Artificial
  Synthetic
FVKLTNEHL

SEQ ID NO: 765 - protein, 9 aa, Artificial
  Synthetic
FVKVTHEHL

SEQ ID NO: 766 - protein, 9 aa, Artificial
  Synthetic
FVKVTNRHL

SEQ ID NO: 767 - protein, 9 aa, Artificial
  Synthetic
FVKVTNEHM

SEQ ID NO: 768 - protein, 9 aa, Artificial
  Synthetic
FVSMTHEHL

SEQ ID NO: 769 - protein, 9 aa, Artificial
  Synthetic
FVSLTHEHL

SEQ ID NO: 770 - protein, 9 aa, Artificial
  Synthetic
FVSVTGEHM

SEQ ID NO: 771 - protein, 9 aa, Artificial
  Synthetic
FVSVTHEHM

SEQ ID NO: 772 - protein, 9 aa, Artificial
  Synthetic
FVSVTNRHM